(19) United States
(12) Patent Application Publication
(10) Pub. No.: US 2022/0380309 A1
(43) Pub. Date: Dec. 1, 2022

(54) CHOLINE METABOLISM INHIBITORS

(71) Applicant: President and Fellows of Harvard College, Cambridge, MA (US)

(72) Inventors: Emily P. Balskus, Somerville, MA (US); Maud Bollenbach, Cambridge, MA (US)

(21) Appl. No.: 17/299,582

(22) PCT Filed: Dec. 4, 2019
(86) PCT No.: PCT/US19/64482

§ 371 Date: Jun. 3, 2021

Related U.S. Application Data
(60) Provisional application No. 62/859,492, filed on Jun. 10, 2019.
(60) Provisional application No. 62/775,164, filed on Dec. 4, 2018.

(51) Int. Cl. C07D 211/74; C07D 409/06; C07D 405/06; C07D 401/06; C07D 409/04

(52) U.S. Cl. CPC C07D 211/74; C07D 409/06; C07D 405/06; C07D 401/06; C07D 409/04

(57) ABSTRACT

The present disclosure relates to compounds, compositions and methods for inhibiting choline metabolism, e.g., conversion of choline to trimethylamine. Disclosed herein are compounds, compositions, and methods for inhibiting choline metabolism, e.g., conversion of choline to TMA. Also disclosed herein are compounds, methods and compositions for inhibiting choline metabolism by gut microbiota resulting in reduction in the formation of trimethylamine (TMA) and trimethylamine N-oxide (TMAO).

### RELATED APPLICATIONS

This application claims the benefit of U.S. Provisional Application No. 62/775,164, filed Dec. 4, 2018, and U.S. Provisional Application No. 62/859,492, filed Jun. 10, 2019, the contents of each application are fully incorporated by reference herein.

### BACKGROUND

The human gut is inhabited by ˜10¹³ symbiotic microorganisms that collectively possess at least 100-fold more genes than the human genome. Changes in the composition of the human gut microbiota have been correlated with a growing number of diseases, strongly suggesting that these organisms and their expanded metabolic capabilities may directly influence disease susceptibility and progression. In support of this scenario, metabolomics studies have shown that levels of metabolites derived from gut bacteria are strongly associated with many diseases, including both non-alcoholic fatty liver disease (NAFLD) and type II diabetes (T2D). The unmet medical need in these areas cannot be overstated. NAFLD is the most common liver condition worldwide, affecting 17% of the total population and 30% of individuals in Western nations, and over 9% of Americans had T2D as of 2015. Therapeutic strategies for reversing NAFLD are currently limited to reducing body fat through caloric restriction and increased physical activity. Similarly, T2D has reached epidemic proportions, and despite the great developments in our arsenal of diabetes treatments, the increased risk of death conferred by diabetes remains unmitigated. Treating liver and cardiometabolic diseases by targeting gut microbial metabolism therefore has enormous therapeutic potential.

Choline is an essential nutrient that lies at an important intersection in host metabolism. Choline is a critical component of the majority of host cell membrane lipids, such as phosphatidylcholine (PC), and choline availability influences liver function and lipid metabolism. It is a precursor to the neurotransmitter acetylcholine and is also oxidized to betaine, a major donor of methyl groups for the synthesis of SAM. Although humans can synthesize choline de novo, dietary choline is also required because this endogenous route typically cannot supply sufficient quantities to support essential cellular processes.

Human gut bacteria metabolize choline in a manner distinct from host cells (FIG. 1). Under anaerobic conditions, they cleave the C—N bond of choline to generate trimethylamine (TMA) and acetaldehyde. While acetaldehyde serves as a carbon and energy source for the bacterium, TMA is a waste product that must be further processed by the host. This exclusively microbial metabolite is transported to the liver and oxidized to trimethylamine-N-oxide (TMAO) by the human liver enzyme flavin monooxygenase 3 (FMO3). TMAO is then excreted from the body in the urine. This interspecies metabolic pathway could substantially alter levels of choline-derived metabolites and thus influence host biology. Indeed, a growing body of evidence links elevated levels of this gut bacterial activity to multiple human diseases.

TMA and its derivative TMAO are metabolites linked to disorders such as kidney disease, diabetes mellitus, trimethylaminuria, and cardiovascular disease (CVD). CVD is a general term encompassing a range of conditions affecting the heart and blood vessels, including atherosclerosis, coronary heart disease, cerebrovascular disease, heart failure, cardiomyopathy, atherothrombotic disease, aorto-iliac disease, and peripheral vascular disease. CVD is generally associated with conditions that involve narrowed, blocked, aneurysmal or dissection of one or more blood vessels, or thrombosis (blood clot formation). Prevention and management of conditions associated with TMA and TMAO, including NAFLD, CVD, T2D, kidney disease and trimethylaminuria, is a major public health concern.

Anaerobic choline metabolism and its role in metabolic disease (NAFLD and T2D): NAFLD—Data from both animal models and patients indicates a strong link between gut microbial choline metabolism and liver disease. Choline deficiency in humans and animal models leads to an accumulation of fat in the liver. Conversely, increasing dietary choline can help to reverse this condition. Given that PC is essential for the synthesis and secretion of very low density lipoprotein (VLDL), reduced lipid transport arising from lowered PC levels could explain the triglyceride accumulation observed in choline deficiency. On a high fat diet, insulin resistant mice developed fatty liver disease and had lower plasma PC levels but dramatically elevated concentrations of TMAO in the urine relative to insulin susceptible controls. Multiple studies indicate an overabundance of choline metabolizing gut bacteria influence both risk for developing NAFLD and disease progression. For example, NAFLD is transmissible to germ-free mice via microbiota transplant. Data from humans also supports a link between the gut microbiota and the development of liver disease. Changes in gut microbiota composition and small intestinal bacterial overgrowth are observed in patients with NAFLD and cirrhosis. In a study of 15 female subjects, the abundance of particular gut bacterial taxa, along with a SNP in a host PC biosynthetic gene, predicted development of fatty liver upon consuming a standardized, choline-deficient diet. Finally, elevated levels of TMAO have been observed in patients with NAFLD.

Recently TMAO was identified as the most highly induced metabolite in livers of insulin resistant mice. Reducing TMAO levels by knocking down the enzyme FMO3 markedly reversed the effects of insulin resistance, entirely preventing hyperglycemia, dyslipidemia, and atherosclerosis; it also lowered hepatic triglycerides by 40% (n=4-8 per group; p<0.01). In addition, TMAO is known to induce PERK activation and the integrated stress response in primary rat hepatocytes. Moreover, TMAO reduces adipocyte browning and energy expenditure and promotes inflammation through activation of NF-κB. Finally, TMAO promotes cardiovascular disease (CVD) and chronic kidney disease (CKD) in mice. These data are exciting for multiple reasons. First, TMAO was identified as an insulin-resistance linked metabolite via nonbiased profiling methods, suggesting it may play a fundamental role in the diabetic state. Second, the fact that TMAO interferes with insulin signaling via FoxO1, promotes ER stress, and drives inflammation indicates this metabolite may be at the center of the molecular pathways coordinately disrupted in diabetes.

Taken together, these data suggest that reducing TMAO may have profound beneficial effects as it would be expected to restore insulin sensitivity and prevent diabetes, as well as its sequelae, CVD, CKD, and NAFLD. As such, there is a need to develop potent and selective small molecule inhibitors of gut bacterial choline utilization, a metabolic process that is connected to NAFLD and T2D.

### SUMMARY OF THE INVENTION

Disclosed herein are compounds, compositions, and methods for inhibiting choline metabolism, e.g., conversion of choline to TMA. Also disclosed herein are compounds, methods and compositions for inhibiting choline metabolism by gut microbiota resulting in reduction in the formation of trimethylamine (TMA) and trimethylamine N-oxide (TMAO).

In one aspect the present disclosure provides compounds of Formula (I), Formula (Ia), Formula (II), or Formula (III):

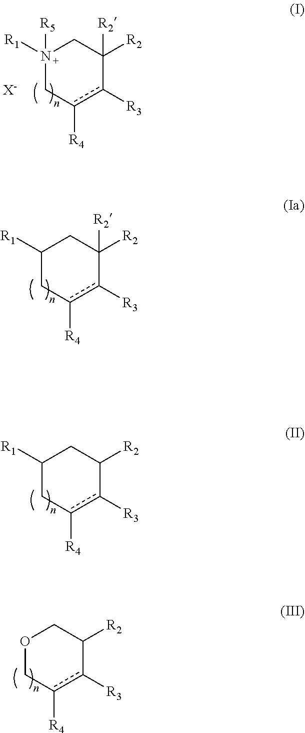

or a pharmaceutically acceptable salt thereof, wherein
[symbol] is a single bond or a double bond;
R₁ is alkyl, cycloalkyl, cycloalkyl(alkyl), heterocyclyl, heterocyclyl(alkyl), aryl, heteroaryl, aralkyl, or heteroaralkyl;
R₂ is hydroxyl, alkoxy, thio, alkylthio, or halo;
R₂′ is H or D;
R₃ is H, alkyl, or cycloalkyl;
R₄ is H, alkyl, or cycloalkyl;
or R₃ and R₄, taken together with the carbon atoms to which they are attached, form a cycloalkyl or cycloalkenyl ring;
R⁵ is alkyl;
X is a counter ion; and
n is 0, 1, 2, or 3.

In one aspect the present disclosure provides compounds of Formula (I′):

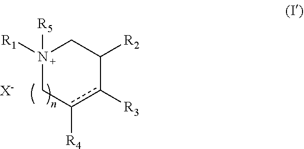

or a pharmaceutically acceptable salt thereof, wherein
[symbol] is a single bond or a double bond;
R₁ is alkyl or cycloalkyl;
R₂ is hydroxyl, alkoxy, thio, alkylthio, or halo;
R₃ is H, alkyl, or cycloalkyl;
R₄ is H, alkyl, or cycloalkyl;
or R₃ and R₄, taken together with the carbon atoms to which they are attached, form a cycloalkyl or cycloalkenyl ring;
R⁵ is alkyl;
X is a counter ion; and
n is 0, 1, 2, or 3.

In one aspect the present disclosure provides compounds of Formula Ia′:

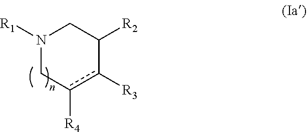

or a pharmaceutically acceptable salt thereof, wherein
[symbol] is a single bond or a double bond;
R₁ is alkyl or cycloalkyl;
R₂ is hydroxyl, alkoxy, thio, alkylthio, or halo;
R₃ is H, alkyl, or cycloalkyl;
R₄ is H, alkyl, or cycloalkyl;
or R₃ and R₄, taken together with the carbon atoms to which they are attached, form a cycloalkyl or cycloalkenyl ring; and
n is 0, 1, 2, or 3,
provided that if R₁ is methyl, R₂ is hydroxyl, [symbol] is a double bond, R₃ is H, and R₄ is H, then n is not 1.

In one aspect the present disclosure provides compounds of Formula II:

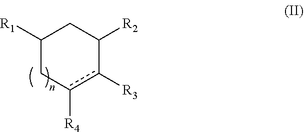

or a pharmaceutically acceptable salt thereof, wherein
[symbol] is a single bond or a double bond;
R₁ is alkyl or cycloalkyl;
R₂ is hydroxyl, alkoxy, thio, alkylthio, or halo;
R₃ is H, alkyl, or cycloalkyl;
R₄ is H, alkyl, or cycloalkyl;
or R₃ and R₄, taken together with the carbon atoms to which they are attached, form a cycloalkyl or cycloalkenyl ring; and
n is 0, 1, 2, or 3.

In one aspect the present disclosure provides compounds of Formula III:

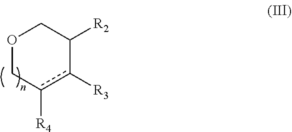

or a pharmaceutically acceptable salt thereof, wherein
[symbol] is a single bond or a double bond;
R₂ is hydroxyl, alkoxy, thio, alkylthio, or halo;
R₃ is H, alkyl, or cycloalkyl;
R₄ is H, alkyl, or cycloalkyl;
or R₃ and R₄, taken together with the carbon atoms to which they are attached, form a cycloalkyl or cycloalkenyl ring; and
n is 0, 1, 2, or 3.

In certain aspects, the present disclosure provides methods inhibiting choline metabolism in a cell. In further aspects, the present disclosure provides methods of inhibiting choline metabolism in a subject.

### BRIEF DESCRIPTION OF THE DRAWINGS

FIG. 1 is a schematic representation of anaerobic microbial choline metabolism in the human gut, its link to disease, and strategy for targeting TMA production with small molecule inhibitors.

### DETAILED DESCRIPTION OF THE INVENTION

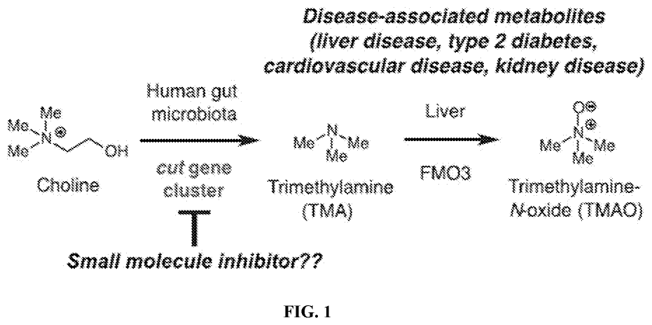

Despite the strong connections to disease, understanding of gut microbial choline metabolism was extremely limited until recently. Previously, the first genes involved in anaerobic bacterial choline metabolism, the choline utilization (cut) gene cluster (FIG. 1), were uncovered. The key TMA-generating enzyme, choline TMA-lyase (CutC), was found to be a new member of the glycyl radical enzyme (GRE) family. The activity, structure, and mechanism of CutC were investigated. The cut genes predict a strain's ability to convert choline to TMA, and cut gene clusters may be found in many human gut isolates. Moreover, a comparative analysis of gut metagenomes from cirrhosis patients and healthy controls revealed increased levels of cutC in subjects with disease relative to healthy controls.

Targeting microbial metabolism is a novel approach to treating NAFLD and T2D that has distinct benefits. First, the direct inhibition of a bacterial enzyme with no human homolog has advantages in terms of specificity and overall safety, and it represents a clear differentiation from all marketed drugs. Second, inhibition of the host enzyme FMO3 is not desirable as TMA accumulation causes severe malodor disease (trimethylaminourea). Finally, as has been seen in antibiotic development, manipulation of microorganisms is associated with a high level of translation from in vitro to in vivo and clinical experiments. The small molecule modality also has distinct advantages for gut microbiota manipulation, including a clearly defined regulatory path through development and approval. Disclosed herein are inhibitors of microbial metabolism, such as choline metabolism.

In one aspect the present disclosure provides compounds of Formula (I), Formula (Ia), Formula (II), or Formula (III):

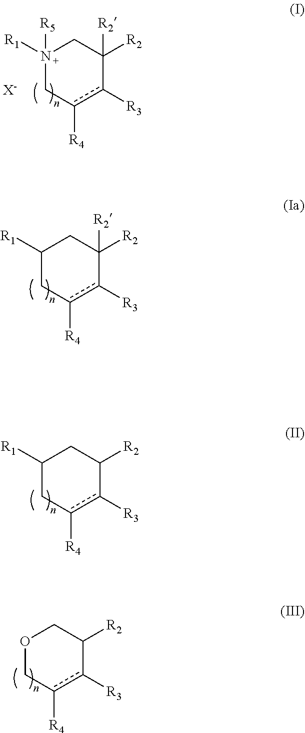

or a pharmaceutically acceptable salt thereof, wherein
[symbol] is a single bond or a double bond;
R₁ is alkyl, cycloalkyl, cycloalkyl(alkyl), heterocyclyl, heterocyclylalkyl, aryl, heteroaryl, aralkyl, or heteroaralkyl;
R₂ is hydroxyl, alkoxy, thio, alkylthio, or halo;
R₂′ is H or D;
R₃ is H, alkyl, or cycloalkyl;
R₄ is H, alkyl, or cycloalkyl;
or R₃ and R₄, taken together with the carbon atoms to which they are attached, form a cycloalkyl or cycloalkenyl ring;
R₅ is alkyl;
X is a counter ion; and
n is 0, 1, 2, or 3.

In one aspect the present disclosure provides compounds of Formula I′:

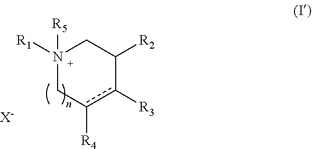

or a pharmaceutically acceptable salt thereof, wherein
[symbol] is a single bond or a double bond;
R₁ is alkyl or cycloalkyl;
R₂ is hydroxyl, alkoxy, thio, alkylthio, or halo;
R₃ is H, alkyl, or cycloalkyl;
R₄ is H, alkyl, or cycloalkyl;
or R₃ and R₄, taken together with the carbon atoms to which they are attached, form a cycloalkyl or cycloalkenyl ring;
R₅ is alkyl;
X is a counter ion; and
n is 0, 1, 2, or 3.

In certain embodiments, if R₁ is methyl, R₂ is hydroxyl, [symbol] is a double bond, R₃ is H, R₄, is H, and R₅ is allyl, then n is not 1.

In certain embodiments, R₅ is methyl.

In certain embodiments, X is halo (e.g., chloro, bromo, or iodo).

In one aspect the present disclosure provides compounds of Formula Ia′:

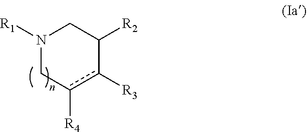

or a pharmaceutically acceptable salt thereof, wherein
[symbol] is a single bond or a double bond;
R₁ is alkyl or cycloalkyl;
R₂ is hydroxyl, alkoxy, thio, alkylthio, or halo;
R₃ is H, alkyl, or cycloalkyl;
R₄ is H, alkyl, or cycloalkyl;
or R₃ and R₄, taken together with the carbon atoms to which they are attached, form a cycloalkyl or cycloalkenyl ring; and
n is 0, 1, 2, or 3,
provided that if R₁ is methyl, R₂ is hydroxyl, [symbol] is a double bond, R₃ is H, and R₄ is H, then n is not 1.

In one aspect the present disclosure provides compounds of Formula II:

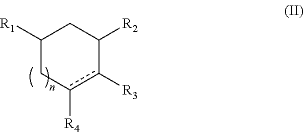

or a pharmaceutically acceptable salt thereof, wherein
[symbol] is a single bond or a double bond;
R₁ is alkyl or cycloalkyl;
R₂ is hydroxyl, alkoxy, thio, alkylthio, or halo;
R₃ is H, alkyl, or cycloalkyl;
R₄ is H, alkyl, or cycloalkyl;
or R₃ and R₄, taken together with the carbon atoms to which they are attached, form a cycloalkyl or cycloalkenyl ring; and
n is 0, 1, 2, or 3.

In one aspect the present disclosure provides compounds of Formula III:

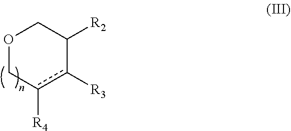

or a pharmaceutically acceptable salt thereof, wherein
[symbol] is a single bond or a double bond;
R₂ is hydroxyl, alkoxy, thio, alkylthio, or halo;
R₃ is H, alkyl, or cycloalkyl;
R₄ is H, alkyl, or cycloalkyl;
or R₃ and R₄, taken together with the carbon atoms to which they are attached, form a cycloalkyl or cycloalkenyl ring; and
n is 0, 1, 2, or 3.

In certain embodiments, the compound is not

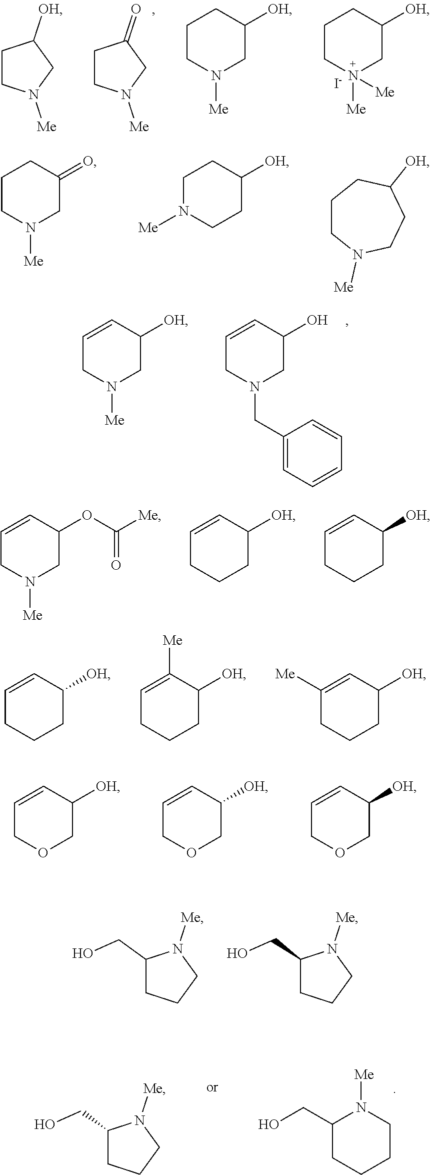

In certain embodiments, the compound is a compound of Formula I.

In certain embodiments, R₁ is alkyl. In some embodiments, R₁ is methyl, ethyl, n-propyl, or i-propyl. In some embodiments, R₁ is haloalkyl, such as haloethyl, for example, 2-chloroethyl. In certain embodiments, R₁ is cycloalkylalkyl, such as cycloalkyl-CH₂—, for example, cyclopropyl-CH₂— or cyclohexyl-CH₂—. In some embodiments, R₁ is heterocyclylalkyl (e.g., tetrahydrothiophenylalkyl). In some embodiments, R₁ is aralkyl or heteroaralkyl, such as benzyl, 4-methoxybenzyl, thiophenyl-CH₂—, furanyl-CH₂—, pyridinyl-CH₂—, or phenyl-CH₂CH₂—. In some embodiments, R₁ is cycloalkyl. In certain preferred embodiments, R₁ is cyclobutyl, cyclopentyl, cyclohexyl, cycloheptyl, or cyclooctyl. In even further preferred embodiment, Riis cyclooctyl. In certain embodiments, Riis substituted with alkyl, carboxyl, hydroxyl, hydroxyalkyl, alkoxy (e.g., methoxy), halogen (e.g., chloro or fluoro), acyl, acyloxy, amino, aminoalkyl, or cyano. In certain embodiments, R₁ is substituted with ester (e.g., methylester). In certain preferred embodiments, R₁ is substituted with alkyl.

In certain, even further preferred embodiments R₁ is substituted with methyl.

In some embodiments, R₂ is hydroxyl, alkoxy, thio, alkylthio. In certain embodiments, R₂ is hydroxyl.

In certain embodiments, [symbol] is a double bond.

In certain embodiments, R₃ is H.

In certain embodiments, R₄ is H.

In some embodiments, [symbol] is a single bond.

In some embodiments, [symbol] is a single bond; and R₃ and R₄, taken together with the carbon atoms to which they are attached, form a cycloalkyl ring, such as a cyclopropyl ring.

In certain preferred embodiments, the carbon connected to R₂ is in the S-configuration. In other preferred embodiments, the carbon connected to R₂ is in the R-configuration.

In certain embodiments, the hydrogen on the carbon connected to R₂ is replaced by deuterium.

In certain embodiments, the compound of Formula (I) is represented by Formula (Ia*), Formula (Ib) or Formula (Ic):

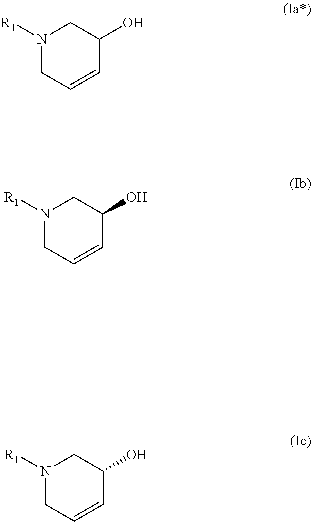

or a pharmaceutically acceptable salt thereof.

In certain embodiments, the compound is a compound of Table 1.

TABLE 1
Exemplary Compounds of the Present Disclosure

| Compound | |
|---|---|
| No. | Structure |
| 1 | 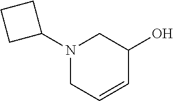 [SMILES: OC1C=CCN(C2CCC2)C1] |
| 2 | 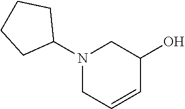 [SMILES: OC1C=CCN(C2CCCC2)C1] |
| 3 | 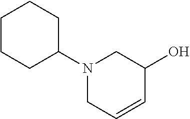 [SMILES: OC1C=CCN(C2CCCCC2)C1] |
| 4 | 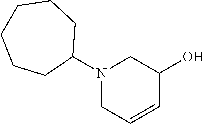 [SMILES: OC1C=CCN(C2CCCCCC2)C1] |
| 5 | 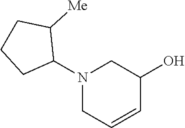 [SMILES: CC1CCCC1N1CC=CC(O)C1] |
| 6 | 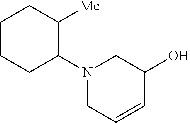 [SMILES: CC1CCCCC1N1CC=CC(O)C1] |
| 7 | 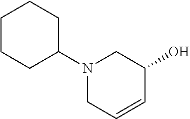 [SMILES: O[C@@H]1C=CCN(C2CCCCC2)C1] |
| 8 | 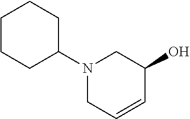 [SMILES: O[C@H]1C=CCN(C2CCCCC2)C1] |
| 9 | 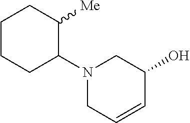 [SMILES: CC1CCCCC1N1CC=C[C@@H](O)C1] |
| 10 | 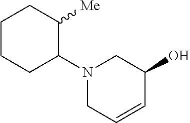 [SMILES: CC1CCCCC1N1CC=C[C@H](O)C1] |
| 11 | 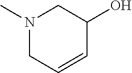 |
| 12 | 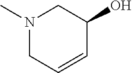 |
| 13 | 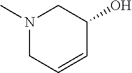 |
| 14 | 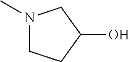 |
| 15 | 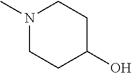 |
| 16 | 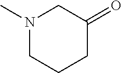 |
| 17 | 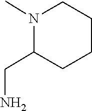 |
| 18A + 18B | 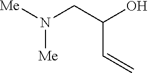 |
| | 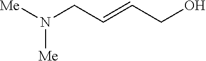 |
| 19 | 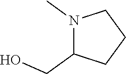 |
| 20 | 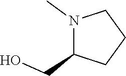 |
| 21 | 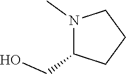 |
| 22 | 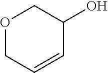 |
| 23 | 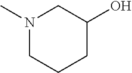 |
| 24 | 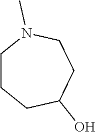 |
| 25 | 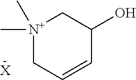 |
| 26 | 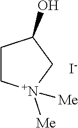 |
| 27 | 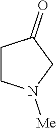 |
| 28 | 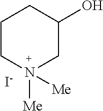 |
| 29 | 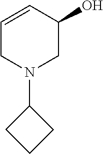 [SMILES: O[C@@H]1C=CCN(C2CCC2)C1] |
| 30 | 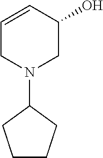 [SMILES: O[C@H]1C=CCN(C2CCCC2)C1] |
| 31 | 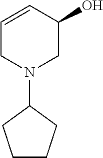 [SMILES: O[C@@H]1C=CCN(C2CCCC2)C1] |
| 32 | 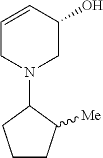 [SMILES: CC1CCCC1N1CC=C[C@H](O)C1] |
| 33 | 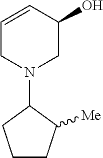 [SMILES: CC1CCCC1N1CC=C[C@@H](O)C1] |
| 34 | 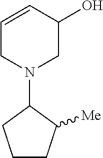 [SMILES: CC1CCCC1N1CC=CC(O)C1] |
| 35 | 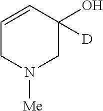 |
| 36 | 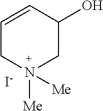 |
| 37 | 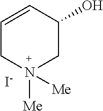 |
| 38 | 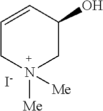 |
| 39 | 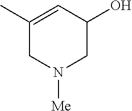 |
| 40 | 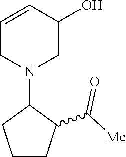 [SMILES: CC(=O)C1CCCC1N1CC=CC(O)C1] |
| 41 | 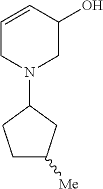 [SMILES: CC1CCC(N2CC=CC(O)C2)C1] |
| 42 | 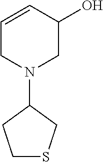 [SMILES: OC1C=CCN(C2CCSC2)C1] |
| 43 | 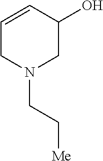 |
| 44 | 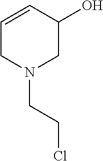 |
| 45 | 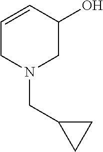 [SMILES: OC1C=CCN(CC2CC2)C1] |
| 46 | 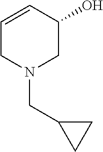 [SMILES: O[C@H]1C=CCN(CC2CC2)C1] |
| 47 | 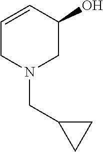 [SMILES: O[C@@H]1C=CCN(CC2CC2)C1] |
| 48 | 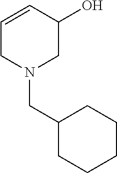 [SMILES: OC1C=CCN(CC2CCCCC2)C1] |
| 49 | 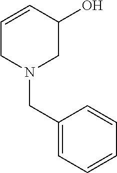 [SMILES: OC1C=CCN(Cc2ccccc2)C1] |
| 50 | 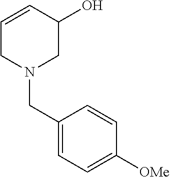 [SMILES: COc1ccc(CN2CC=CC(O)C2)cc1] |
| 51 | 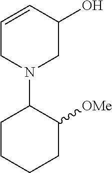 [SMILES: COC1CCCCC1N1CC=CC(O)C1] |
| 52 | 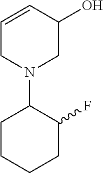 [SMILES: OC1C=CCN(C2CCCCC2F)C1] |
| 53 | 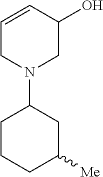 [SMILES: CC1CCCC(N2CC=CC(O)C2)C1] |
| 54 | 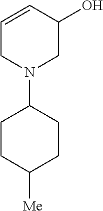 [SMILES: CC1CCC(N2CC=CC(O)C2)CC1] |
| 55 | 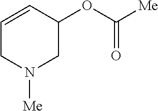 [SMILES: CC(=O)OC1C=CCN(C)C1] |
| 56 | 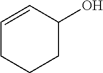 |
| 57 | 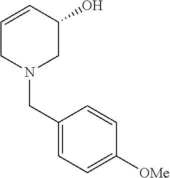 [SMILES: COc1ccc(CN2CC=C[C@H](O)C2)cc1] |
| 58 | 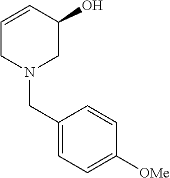 [SMILES: COc1ccc(CN2CC=C[C@@H](O)C2)cc1] |
| 59 | 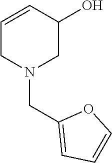 [SMILES: OC1C=CCN(Cc2ccco2)C1] |
| 60 | 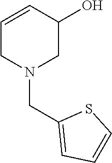 [SMILES: OC1C=CCN(Cc2cccs2)C1] |
| 61 | 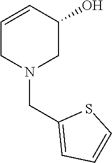 [SMILES: O[C@H]1C=CCN(Cc2cccs2)C1] |
| 62 | 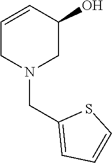 [SMILES: O[C@@H]1C=CCN(Cc2cccs2)C1] |
| 63 | 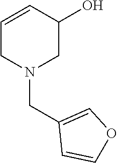 [SMILES: OC1C=CCN(Cc2ccoc2)C1] |
| 64 | 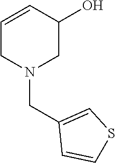 [SMILES: OC1C=CCN(Cc2ccsc2)C1] |
| 65 | 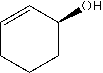 |
| 66 | 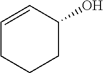 |
| 67 | 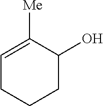 |
| 68 | 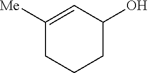 |
| 69 | 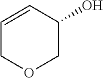 |
| 70 | 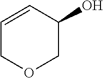 |
| 71 | 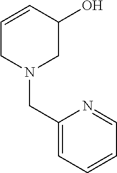 [SMILES: OC1C=CCN(Cc2ccccn2)C1] |
| 72 | 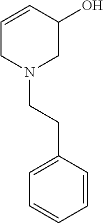 [SMILES: OC1C=CCN(CCc2ccccc2)C1] |
| 73 | 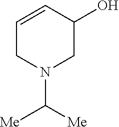 |
| 74 | 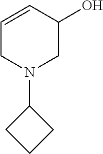 [SMILES: OC1C=CCN(C2CCC2)C1] |
| 75 | 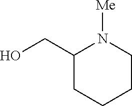 |
| — | — |

wherein is a counter ion.

In certain embodiments, the compound is a pharmaceutically acceptable salt of a compound of Table 1.

In certain embodiments, the compound is

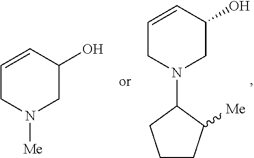 [SMILES: CN1CC=CC(O)C1 ; CC1CCCC1N1CC=C[C@H](O)C1]

or a pharmaceutically acceptable salt thereof.

In certain embodiments, the compound is

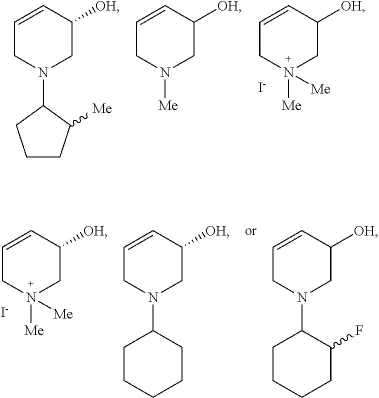

or a pharmaceutically acceptable salt thereof.

In certain embodiments, the compound is

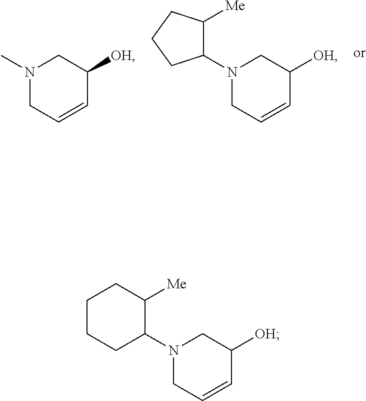

or a pharmaceutically acceptable salt thereof.

In certain embodiments, the compound is not

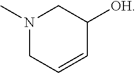

In another aspect, the present disclosure provides a pharmaceutical composition comprising a compound of the disclosure and at least one pharmaceutically acceptable excipient.

In certain embodiments, the pharmaceutical composition comprises a compound having the following structure:

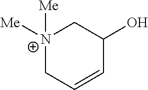

and at least one pharmaceutically acceptable excipient.

In certain embodiments, the pharmaceutical composition comprises a compound having the following structure:

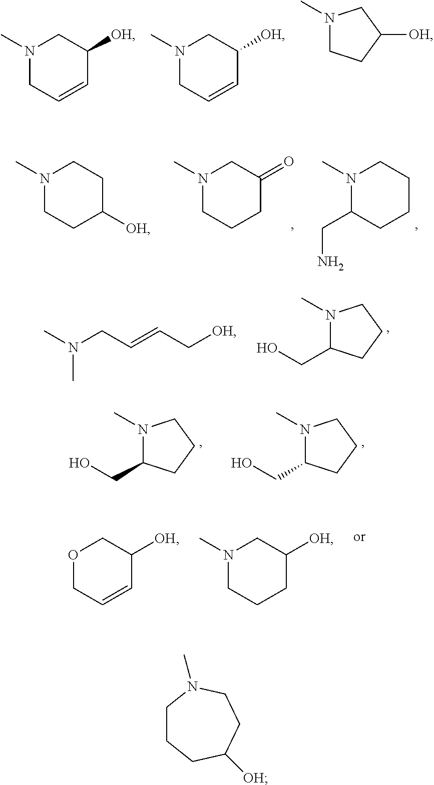

and at least one pharmaceutically acceptable excipient.

In certain embodiments, the pharmaceutical composition comprises a compound having the following structure:

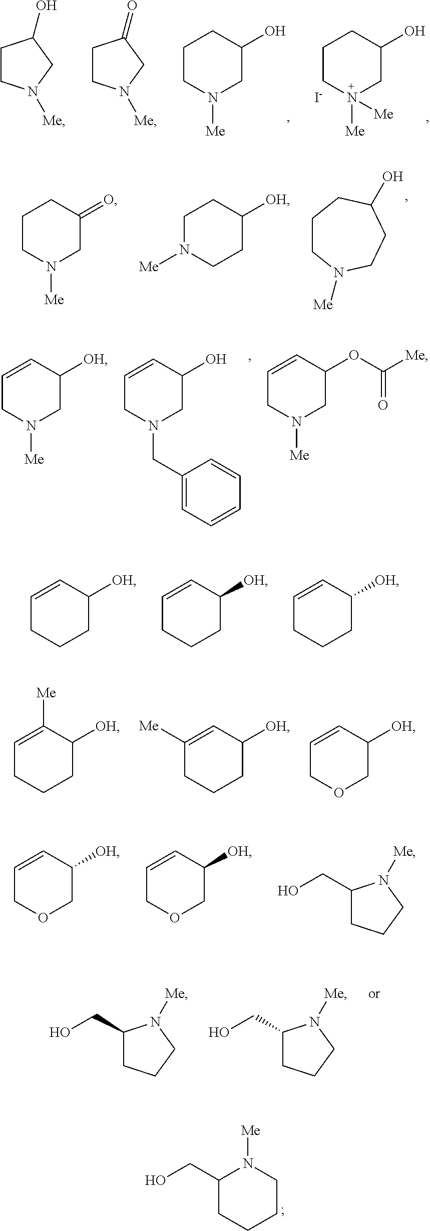

and at least one pharmaceutically acceptable excipient.

In yet another aspect, the present disclosure provides a method for inhibiting choline metabolism in a cell, comprising contacting a cell with a compound or composition disclosed herein. In certain embodiments, the cell is a choline metabolizing microbe cell. In certain embodiments, the microbe is selected from E. coli, Proteus mirabilis, C. sporogenes, A. hydrogenalis, Desulfovibrio alaskensis, Clostridium ljungdahlii, C. scindens, C. aldenense, Collinsella tanakaei, Anaerococcus vaginalis, Streptococcus dysgalactiae, Desultitobacterium hafniense, Klebsiella variicola, Klebsiella sp., and K. pneumonia.

In certain embodiments, the compound inhibits conversion of choline to trimethylamine (TMA). In certain embodiments, TMA production is reduced by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% reduction; or from about 1% to about 100%, about 10% to about 90%, about 20% to about 80%, about 30% to about 70% or about 40% to about 60%.

In certain embodiments, the cell expresses a microbial cut gene cluster. In certain embodiments, the microbial cut gene cluster encodes choline TMA-lyase (CutC).

In yet another aspect, the present disclosure provides, a method for inhibiting choline metabolism in a subject, comprising administering to the subject an effective amount of a compound, composition or pharmaceutically acceptable salt of a compound disclosed herein. In certain embodiments, the compound inhibits conversion of choline to trimethylamine (TMA). In certain embodiments, TMA production is reduced by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% reduction; or from about 1% to about 100%, about 10% to about 90%, about 20% to about 80%, about 30% to about 70% or about 40% to about 60%.

In certain embodiments, the subject identified as having a choline disorder. In certain embodiments, the disorder is associated with TMA to TMAO metabolism. In certain embodiments, the method further comprises diagnosing a subject with a choline disorder. In certain embodiments, the method further comprises detecting the presence of a cut gene cluster in a gut microbial sample from the subject. In certain embodiments, the method further comprises determining the choline, TMA, TMAO level in a sample from the subject.

In yet another aspect, the present disclosure provides, a method of treating a choline choline disorder in a subject, comprising administering to the subject an effective amount of a compound, composition or pharmaceutically acceptable salt of a compound disclosed herein. In certain embodiments, the method further comprises detecting the presence of a cut gene cluster in a gut microbial sample from the subject. In certain embodiments, the method further comprises determining the choline, TMA, TMAO level in a sample from the subject.

In certain embodiments, the choline disorder is selected from liver disease, a metabolic disease, a neurological disorder, and gut dysbiosis. In certain embodiments, the liver disease is selected from al anti-trypsin deficiency, autoimmune hepatitis, biliary atresia, cirrhosis, non-alcoholic fatty liver disease (NAFLD), fatty liver disease, galactosemia, gallstones, Gilbert's syndrome, hemochromatosis, liver cancer, lysosomal acid lipase deficiency, primary biliary cholangitis, porphyria, Reye's syndrome, sarcoidosis, toxic hepatitis, type 1 glycogen storage disease, tyrosinemia, viral hepatitis A, viral hepatitis B, viral hepatitis C, and Wilson disease. In certain embodiments, the liver disease is non-alcoholic fatty liver disease or cirrhosis.

In certain embodiments, the metabolic disease is selected from trimethylaminuria, a lysosomal storage disorder, a glycogen storage disease, a mitochondrial disorder, Friedreich ataxia, a peroxisomal disorder, a metal metabolism disorder, organic acidemias, a mucopolysaccharide disorder, and a urea cycle disease or disorder. In certain embodiments, the metabolic disease is type 2 diabetes, chronic kidney disease, or cardiovascular disease. In certain embodiments, the choline disorder is type 2 diabetes.

In certain embodiments, the neurological disorder is selected from depression, anxiety, schizophrenia, Alzheimer's disease, obsessive compulsive disorder, attention deficit disorder, and attention deficit hyperactivity disorder.

In certain embodiments, the method treats the choline disorder.

In certain embodiments, the method further comprises administering at least one additional therapeutic agent.

In certain embodiments, the therapeutic agent is an agent that treats a metabolic disorder.

Any suitable method for measuring TMA in vitro or in vivo can be used in the context of the invention. TMA, metabolites of TMA (e.g., TMAO, dimethylamine (DMA), or methylamine (MA)), stable isotopes of TMA (e.g., deuterium labeled TMA, such as d3-, d6-, or d9-TMA), stable isotopes of TMAO (e.g., deuterium labeled TMAO, such as d3-, d6-, or d9-TMAO), stable isotopes of DMA (e.g., deuterium labeled DMA, such as d3-, or d6-DMA), stable isotopes of MA (e.g., deuterium labeled MA, such as d3-MA), and/or choline (including stable isotopes of choline, for example d9-choline) can be assessed quantitatively or qualitatively. Exemplary methods of detecting and quantifying TMA are described in, e.g., U.S. Pub. No. 2010/00285517, the contents of which is incorporated herein by reference in its entirety. For example, levels of TMA (or trimethylamine-N-oxide (TMAO), DMA, or MA) and/or choline are optionally measured via mass spectrometry, ultraviolet spectroscopy, or nuclear magnetic resonance spectroscopy. Mass spectrometers include an ionizing source (e.g., electrospray ionization), an analyzer to separate the ions formed in the ionization source according to their mass-to-charge (m/z) ratios, and a detector for the charged ions. In tandem mass spectrometry, two or more analyzers are included. Such methods are standard in the art and include, for example, HPLC with on-line electrospray ionization (ESI) and tandem mass spectrometry.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl,

—OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

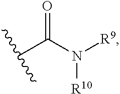

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

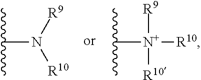

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

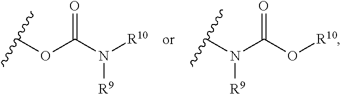

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”.

Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

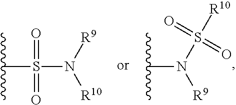

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds.

The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹, wherein R⁹ represents a hydrocarbyl.

The term “thioether”, as used herein, is equivalent to an ether, wherein the oxygen is replaced with a sulfur.

The term “urea” is art-recognized and may be represented by the general formula

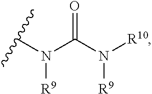

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I, Formula II, or Formula III. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I, Formula II, or Formula III are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I, Formula II, or Formula III for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I, Formula II, or Formula III or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I, formula II, or formula III). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I, Formula II, or Formula III. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

### EXAMPLES

The invention now being generally described, it will be more readily understood by reference to the following examples which are included merely for purposes of illustration of certain aspects and embodiments of the present invention, and are not intended to limit the invention.

### Example 1: Preparation of Exemplary Compounds

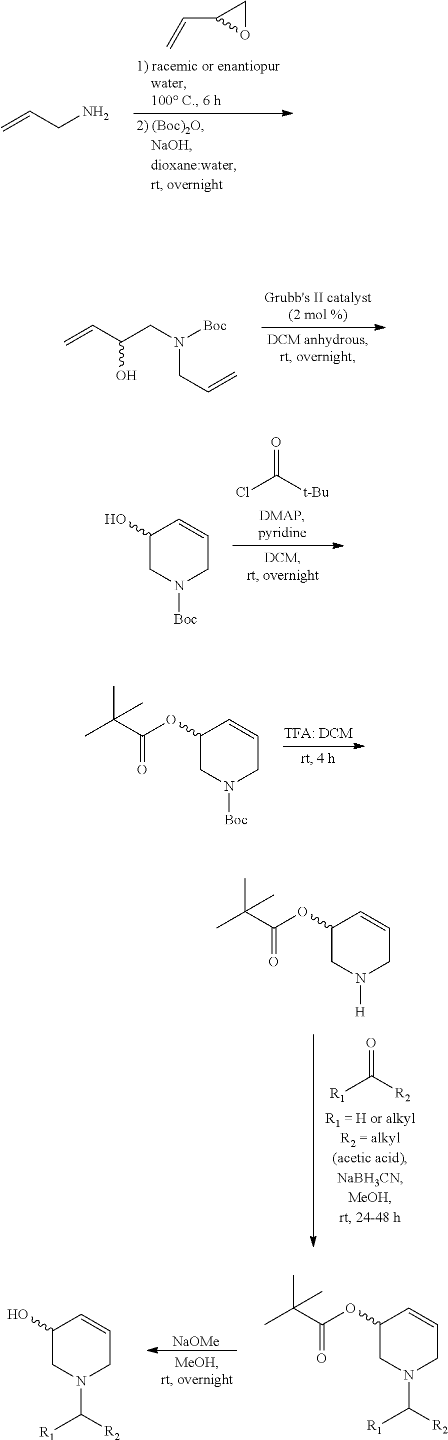

### Example 2: In Vivo Test of Exemplary Compounds—Inhibition of TMA Production in Whole Cells (E. Coli MS 200-1)

Experimental set-up: Inhibitors were added to BHI supplemented with 1 mM choline-d9 and inoculated with 2% of an overnight culture grown to saturation in BHI containing 1 mM choline. The cultures were incubated anaerobically for 3.5 hrs at 37° C. in 96-well plates covered with an aluminum seal. An aliquot of each culture was diluted into LC-MS solvent and analyzed by LC-MS/MS for TMA-d9 production.

TABLE 2
Exemplary Activity Compounds of the Present Disclosure

| Compound No. | EC₅₀ (μM) | Compound No. | EC₅₀ (μM) |
|---|---|---|---|
| 1 | 154 | 14 | 6,300 |
| 2 | 54 | 15 | 4,400 |
| 3 | 20 | 16 | 9,500 |
| 4 | 24 | 17 | 120 |
| 5 | 15 | 18A + 18B | 41,000 |
| 6 | 2 | 19 | 250 |
| 7 | 108 | 20 | 280 |
| 8 | 14 | 21 | 2,040 |
| 9 | 68 | 22 | 25 |
| 10 | 1.2 | 23 | 950 |
| 11 | 10 | 24 | 760 |
| 12 | 10 | 25 | 10 |
| 13 | 15 | — | — |

TABLE 3
Exemplary Activity Compounds of the Present Disclosure against
TMA generation of E. coli MS200-1.

| | EC₅₀ |
|---|---|
| | against |
| | E. coli |
| | MS200-1 |
| Structure | (μM) |
| 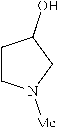 | 6,300 |
| 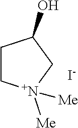 | 2,100 |
| 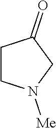 | >30,000 |
| 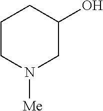 | 950 |
| 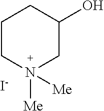 | >30,000 |
| 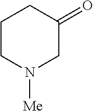 | 27,002 |
| 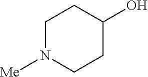 | 4,469 |
| 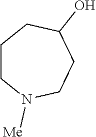 | 762 |
| 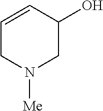 | 10 |
| 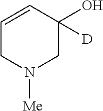 | 30 |
| 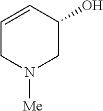 | 10 |
| 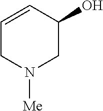 | 31 |
| 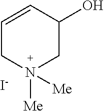 | 8 |
| 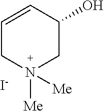 | 9 |
| 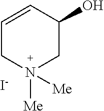 | 22 |
| 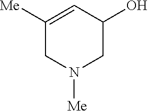 | 923 |
| 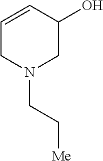 | 196 |
| 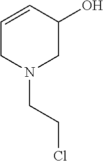 | 113 |
| 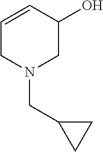 [SMILES: OC1C=CCN(CC2CC2)C1] | 252 |
| 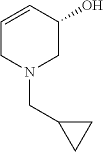 [SMILES: O[C@H]1C=CCN(CC2CC2)C1] | 319 |
| 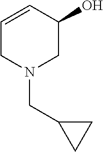 [SMILES: O[C@@H]1C=CCN(CC2CC2)C1] | 151 |
| 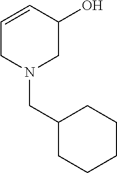 [SMILES: OC1C=CCN(CC2CCCCC2)C1] | 91 |
| 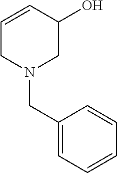 [SMILES: OC1C=CCN(Cc2ccccc2)C1] | 367 |
| 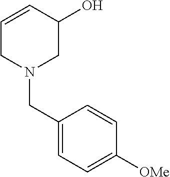 [SMILES: COc1ccc(CN2CC=CC(O)C2)cc1] | 304 |
| 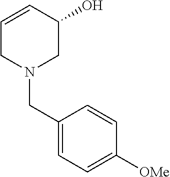 [SMILES: COc1ccc(CN2CC=C[C@H](O)C2)cc1] | 383 |
| 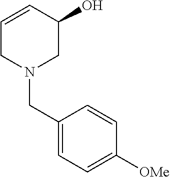 [SMILES: COc1ccc(CN2CC=C[C@@H](O)C2)cc1] | 141 |
| 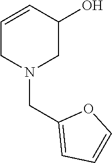 [SMILES: OC1C=CCN(Cc2ccco2)C1] | 197 |
| 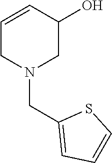 [SMILES: OC1C=CCN(Cc2cccs2)C1] | 225 |
| 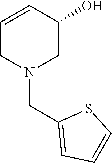 [SMILES: O[C@H]1C=CCN(Cc2cccs2)C1] | 191 |
| 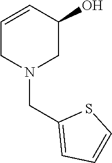 [SMILES: O[C@@H]1C=CCN(Cc2cccs2)C1] | 213 |
| 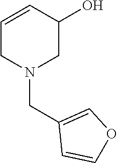 [SMILES: OC1C=CCN(Cc2ccoc2)C1] | 256 |
| 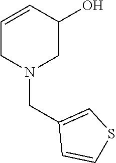 [SMILES: OC1C=CCN(Cc2ccsc2)C1] | 260 |
| 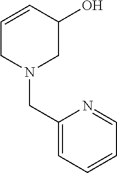 [SMILES: OC1C=CCN(Cc2ccccn2)C1] | 96% inhibition at 3 mM |
| 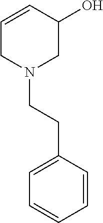 [SMILES: OC1C=CCN(CCc2ccccc2)C1] | 294 |
| 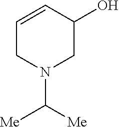 | 83% inhibition at 3 mM |
| 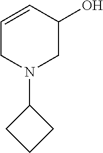 [SMILES: OC1C=CCN(C2CCC2)C1] | 192 |
| 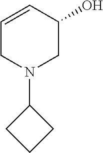 [SMILES: O[C@H]1C=CCN(C2CCC2)C1] | 116 |
| 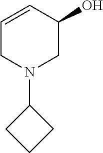 [SMILES: O[C@@H]1C=CCN(C2CCC2)C1] | 193 |
| 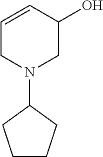 [SMILES: OC1C=CCN(C2CCCC2)C1] | 42 |
| 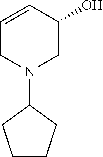 [SMILES: O[C@H]1C=CCN(C2CCCC2)C1] | 41 |
| 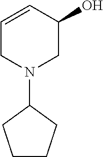 [SMILES: O[C@@H]1C=CCN(C2CCCC2)C1] | 62 |
| 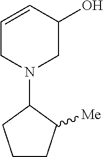 [SMILES: CC1CCCC1N1CC=CC(O)C1] | 15 |
| 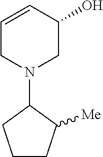 [SMILES: CC1CCCC1N1CC=C[C@H](O)C1] | 9 |
| 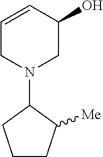 [SMILES: CC1CCCC1N1CC=C[C@@H](O)C1] | 164 |
| 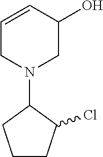 [SMILES: OC1C=CCN(C2CCCC2Cl)C1] | 52 |
| 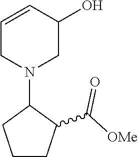 [SMILES: COC(=O)C1CCCC1N1CC=CC(O)C1] | 62% inhibition at 1 mM |
| 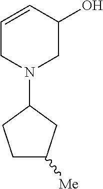 [SMILES: CC1CCC(N2CC=CC(O)C2)C1] | 49 |
| 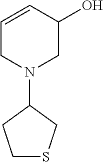 [SMILES: OC1C=CCN(C2CCSC2)C1] | 173 |
| 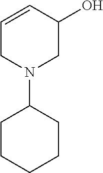 [SMILES: OC1C=CCN(C2CCCCC2)C1] | 22 |
| 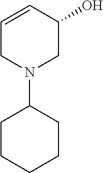 [SMILES: O[C@H]1C=CCN(C2CCCCC2)C1] | 14 |
| 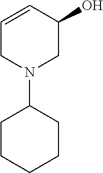 [SMILES: O[C@@H]1C=CCN(C2CCCCC2)C1] | 108 |
| 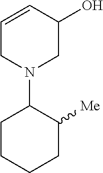 [SMILES: CC1CCCCC1N1CC=CC(O)C1] | 2 |
| 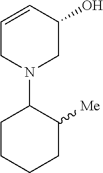 [SMILES: CC1CCCCC1N1CC=C[C@H](O)C1] | 12 |
| 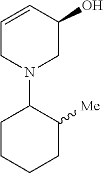 [SMILES: CC1CCCCC1N1CC=C[C@@H](O)C1] | 68 |
| 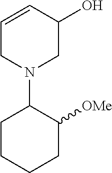 [SMILES: COC1CCCCC1N1CC=CC(O)C1] | 40% inhibition at 100 μM |
| 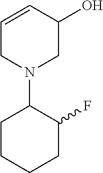 [SMILES: OC1C=CCN(C2CCCCC2F)C1] | 12 |
| 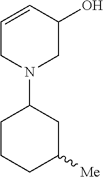 [SMILES: CC1CCCC(N2CC=CC(O)C2)C1] | 17 |
| 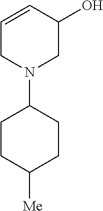 [SMILES: CC1CCC(N2CC=CC(O)C2)CC1] | 42 |
| 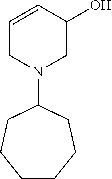 [SMILES: OC1C=CCN(C2CCCCCC2)C1] | 24 |
| 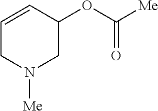 [SMILES: CC(=O)OC1C=CCN(C)C1] | 1,788 |
| 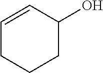 | 259 |
| 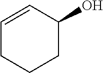 | 404 |
| 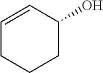 | 482 |
| 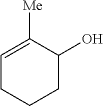 | 3,803 |
| 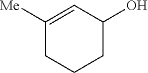 | 14,080 |
| 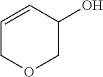 | 80 |
| 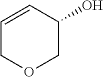 | 112 |
| 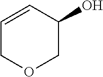 | 84 |
| 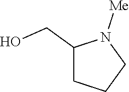 | 298 |
| 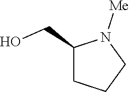 | 185 |
| 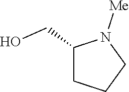 | 1,819 |
| 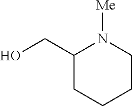 | 120 |
| 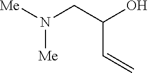 | 70 |
| 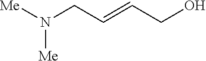 | |
| / | / |

TABLE 4
Exemplary Activity Compounds of the Present Disclosure against CutC

| | IC₅₀ |
|---|---|
| | CutC |
| Structure | (μM) |
| 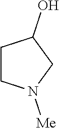 | 844 |
| 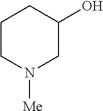 | 2,046 |
| 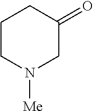 | 20% inhibition at 10 mM |
| 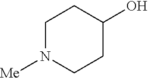 | 7,007 |
| 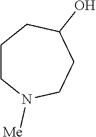 | 2,602 |
| 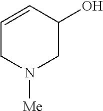 | 3 |
| 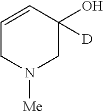 | 6 |
| 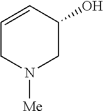 | 2 |
| 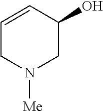 | 22 |
| 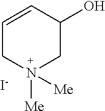 | 7 |
| 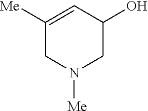 | 422 |
| 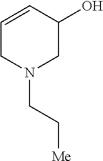 | 141 |
| 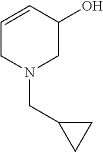 [SMILES: OC1C=CCN(CC2CC2)C1] | 76 |
| 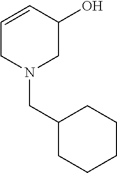 [SMILES: OC1C=CCN(CC2CCCCC2)C1] | 24 |
| 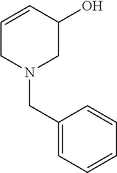 [SMILES: OC1C=CCN(Cc2ccccc2)C1] | 100 |
| 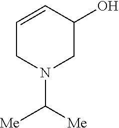 | 110 |
| 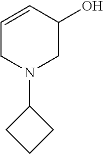 [SMILES: OC1C=CCN(C2CCC2)C1] | 203 |
| 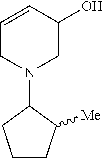 [SMILES: CC1CCCC1N1CC=CC(O)C1] | No inhibition at 10 μM |
| 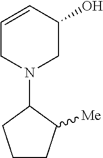 [SMILES: CC1CCCC1N1CC=C[C@H](O)C1] | No inhibition at 10 μM |
| 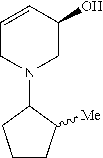 [SMILES: CC1CCCC1N1CC=C[C@@H](O)C1] | No inhibition at 10 μM |
| 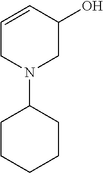 [SMILES: OC1C=CCN(C2CCCCC2)C1] | 63 |
| 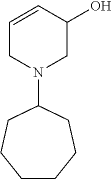 [SMILES: OC1C=CCN(C2CCCCCC2)C1] | 14 |
| 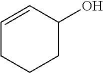 | 709 |
| 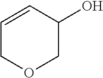 | 2,891 |
| 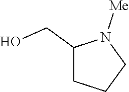 | 513 |
| 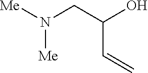 | 23 |
| 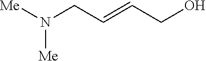 | |

TABLE 5
Exemplary Activity Compounds of the Present Disclosure against TMA
generation of E. coli MS69-1, P. mirabilis ATCC 29906, Klebsiella sp.
MS92-3, C. sporogenes ATCC 15579 and fecal sample

| | | EC₅₀ | | EC₅₀ | |
|---|---|---|---|---|---|
| | EC₅₀ | against P. | EC₅₀ | against C. | EC₅₀ |
| | against | mirabilis | against | sporogenes | against |
| | E. coli | ATCC | Klebsiella | ATCC | fecal |
| | MS69-1 | 29906 | sp. MS92- | 15579 | sample |
| Structure | (μM) | (μM) | 3 (μM) | (μM) | (μM) |
| 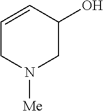 | 27 | 97 | 5 | 12 | 78 |
| 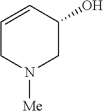 | 22 | 92 | 4 | 12 | 60 |
| 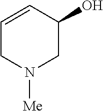 | 64 | 366 | 17 | 71 | / |

### Example 3: Synthetic Protocols

Intramolecular metathesis. General procedure A: In an oven-dried round-bottom flask under argon, the reactant (1.0 equiv.) was dissolved in anhydrous DCM (0.06 M) and Grubbs 2^{nd} generation (2 mol %) was added. The resulting mixture was stirred at room temperature overnight. The reaction mixture was concentrated under vacuum. The crude was purified by chromatography on silica gel, using the appropriate hexanes:EtOAc mixture.

Boc deprotection. General procedure B: The protected amine (1.0 equiv.) was dissolved in DCM (0.35 M) and cooled down at 0° C. TFA (14.6 equiv.) was added dropwise and the resulting mixture was stirred at room temperature for 4-6 hours. The reaction mixture was concentrated under vacuum. The residue was resuspended in EtOAc. The organic phase was washed twice with a saturated solution of NaHCO₃ and brine, dried over MgSO₄ and concentrated under vacuum.

Reductive amination using acetic acid. General procedure C: In an oven-dried round-bottom flask under argon, the free amine (1.0 equiv.) was dissolved in anhydrous methanol (0.18 M) and cooled down at 0° C. The aldehyde/ketone (3.0-5.0 equiv.) and acetic acid (1.0 equiv.) were added and stirred at 0° C. for 30 minutes. NaBH₃CN (2.5 equiv.) was added and the resulting reaction mixture was stirred at room temperature overnight. The reaction mixture was concentrated under vacuum. The residue was resuspended in water and the aqueous phase was extracted 3 times with EtOAc. The combined organic layers were washed with brine, dried over MgSO₄ and concentrated under vacuum. The crude was purified by chromatography on silica gel, using the appropriate hexanes:EtOAc mixture.

Reductive amination. General procedure D: In an oven-dried round-bottom flask under argon, the free amine (1.0 equiv.) was dissolved in anhydrous methanol (0.18 M) and cooled down at 0° C. The aldehyde/ketone (3.0 equiv.) was added and stirred at 0° C. for 30 minutes.

NaBH₃CN (2.5 equiv.) was added and the resulting reaction mixture was stirred at room temperature overnight. The reaction mixture was concentrated under vacuum. The residue was resuspended in water and the aqueous phase was extracted 3 times with EtOAc. The combined organic layers were washed with brine, dried over MgSO₄ and concentrated under vacuum. The crude was purified by chromatography on silica gel, using the appropriate hexanes:EtOAc mixture.

Trimethylacetyl deprotection. General procedure E: In an oven-dried round-bottom flask under argon, the reactant (1.0 equiv.) was dissolved in anhydrous methanol (0.76 M). A 25% solution of sodium methoxide in methanol (5.0 equiv.) was added and the resulting mixture was stirred at room temperature overnight. The reaction mixture was concentrated under vacuum. The residue was purified by chromatography on silica gel, using the appropriate EtOAc:methanol:NH₄OH 28% mixture.

### Preparation of Certain Compounds

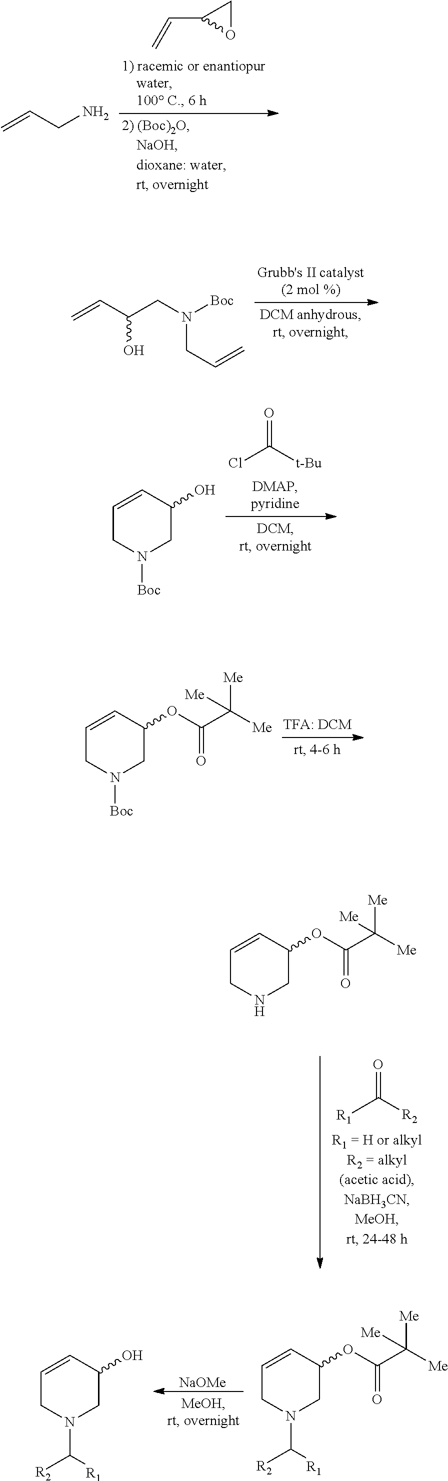

### Step 1. Formation of tert-butyl allyl(2-hydroxybut-3-en-1-yl)carbamate

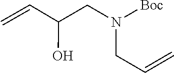 [SMILES: C=CCN(CC(O)C=C)C(=O)OC(C)(C)C]

To a solution of allylamine (5.6 mL, 74.50 mmol, 3.0 equiv) and water (358 μL, 19.86 mmol, 0.8 equiv) was added 2-vinyloxirane (2.0 mL, 24.82 mmol, 1.0 equiv.). The mixture was heated at 80° C. for 6 hours. Once cooled down, the reaction mixture was concentrated under vacuum. The residue was dissolved in a mixture dioxane:water (1:1, 66 mL, 0.37 M). A 50% solution of NaOH (2.6 mL, 49.60 mmol, 2.0 equiv.) and di-tert-butyl decarbonate (10.8 g, 49.60 mmol, 2.0 equiv.) were added and the resulting mixture was stirred at room temperature overnight. The reaction mixture was diluted with EtOAc and washed twice with a 20% citric acid solution and then brine. The organic layer was dried over MgSO₄ and concentrated under vacuum, to afford tert-butyl allyl(2-hydroxybut-3-en-1-yl)carbamate as a colorless oil (5.64 g, 24.82 mmol, 100%). ¹H NMR (400 MHz, CDCl₃) δ ppm 1.45 (s, 9H), 3.29 (br s, 2H), 3.87 (br s, 2H), 4.32 (q, 1H, J=5.6 Hz), 5.10-5.19 (m, 3H), 5.33 (d, 1H, J=17.3 Hz), 5.75-5.90 (m, 2H).

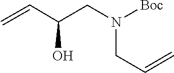 [SMILES: C=CCN(C[C@@H](O)C=C)C(=O)OC(C)(C)C]

To a solution of allylamine (5.0 mL, 67.00 mmol, 3.0 equiv) and water (322 μL, 17.87 mmol, 0.8 equiv) was added butadiene monoxide (S)-2-vinyloxirane (1.8 mL, 22.34 mmol, 1.0 equiv.). The mixture was heated at 80° C. for 6 hours. Once cooled down, the reaction mixture was concentrated under vacuum. The residue was dissolved in a mixture dioxane:water (1:1, 60 mL, 0.37 M). A 50% solution of NaOH (2.3 mL, 44.70 mmol, 2.0 equiv.) and di-tert-butyl decarbonate (9.8 g, 44.70 mmol, 2.0 equiv.) were added and the resulting mixture was stirred at room temperature overnight. The reaction mixture was diluted with EtOAc and washed twice with a 20% citric acid solution and then brine. The organic layer was dried over MgSO₄ and concentrated under vacuum, to afford tert-butyl (S)-allyl(2-hydroxybut-3-en-1-yl)carbamate as a colorless oil (5.64 g, 24.82 mmol, 100%). ¹H NMR (400 MHz, CDCl₃) δ ppm 1.46 (s, 9H), 3.28 (br s, 2H), 3.87 (br s, 2H), 4.32 (q, 1H, J=5.6 Hz), 5.10-5.19 (m, 3H), 5.33 (d, 1H, J=17.2 Hz), 5.75-5.90 (m, 2H).

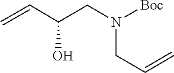 [SMILES: C=CCN(C[C@H](O)C=C)C(=O)OC(C)(C)C]

To a solution of allylamine (1.9 mL, 26.50 mmol, 3.0 equiv) and water (127 μL, 7.05 mmol, 0.8 equiv) was added butadiene monoxide (R)-2-vinyloxirane (618 mg, 8.82 mmol, 1.0 equiv.). The mixture was heated at 80° C. for 6 hours. Once cooled down, the reaction mixture was concentrated under vacuum. The residue was dissolved in a mixture dioxane:water (1:1, 24 mL, 0.37 M). A 50% solution of NaOH (931 μL, 17.63 mmol, 2.0 equiv.) and di-tert-butyl decarbonate (1.4 g, 17.63 mmol, 2.0 equiv.) were added and the resulting mixture was stirred at room temperature overnight. The reaction mixture was diluted with EtOAc and washed twice with a 20% citric acid solution and then brine. The organic layer was dried over MgSO₄ and concentrated under vacuum, to afford tert-butyl (R)-allyl(2-hydroxybut-3-en-1-yl)carbamate as a colorless oil (5.64 g, 24.82 mmol, 100%). ¹H NMR (400 MHz, CDCl₃) δ ppm 1.46 (s, 9H), 3.28 (br s, 2H), 3.87 (br s, 2H), 4.32 (q, 1H, J=5.5 Hz), 5.10-5.19 (m, 3H), 5.34 (d, 1H, J=17.0 Hz), 5.75-5.90 (m, 2H).

### Step 2. Formation of tert-butyl 3-hydroxy-3,6-dihydropyridine-1(2H)-carboxylate

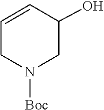 [SMILES: CC(C)(C)OC(=O)N1CC=CC(O)C1]

Following general procedure A and starting from tert-butyl allyl(2-hydroxybut-3-en-1-yl)carbamate (7.0 g, 31.00 mmol, 1.0 equiv.), tert-butyl 3-hydroxy-3,6-dihydropyridine-1(2H)-carboxylate was obtained as a brown oil (5.3 g, 26.90 mmol, 87%). ¹H NMR (400 MHz, CDCl₃) δ ppm 1.47 (s, 9H), 3.50 (dd, 1H, J=13.3 Hz, J=5.3 Hz), 3.60 (dd, 1H, J=13.2 Hz, J=4.0 Hz), 3.79 (dq, 1H, J=18.8 Hz, J=2.2 Hz), 3.97 (d, 1H, J=18.8 Hz), 4.19 (br s, 1H), 5.82 (dt, 1H, J=9.9 Hz, J=2.7 Hz), 5.91 (dq, 1H, J=10.2 Hz, J=3.2 Hz).

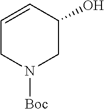 [SMILES: CC(C)(C)OC(=O)N1CC=C[C@H](O)C1]

Following general procedure A and starting from tert-butyl (S)-allyl(2-hydroxybut-3-en-1-yl)carbamate) (8.7 g, 38.40 mmol, 1.0 equiv.), tert-butyl (S)-3-hydroxy-3,6-dihydropyridine-1(2H)-carboxylate was obtained as a brown oil (5.2 g, 26.00 mmol, 68%). ¹H NMR (400 MHz, CDCl₃) δ ppm 1.47 (s, 9H), 3.53 (d, 1H, J=13.3 Hz), 3.58 (d, 1H, J=13.2 Hz), 3.79 (dq, 1H, J=19.0 Hz, J=2.2 Hz), 3.98 (d, 1H, J=18.1 Hz), 4.19 (br s, 1H), 5.82 (d, 1H, J=9.8 Hz), 5.91 (dq, 1H, J=10.2 Hz, J=3.1 Hz).

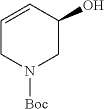 [SMILES: CC(C)(C)OC(=O)N1CC=C[C@@H](O)C1]

Following general procedure A and starting from tert-butyl (R)-allyl(2-hydroxybut-3-en-1-yl)carbamate (8.1 g, 35.70 mmol, 1.0 equiv.), tert-butyl (R)-3-hydroxy-3,6-dihydropyridine-1(2H)-carboxylate was obtained as a brown oil (4.82 g, 24.09 mmol, 68%). ¹H NMR (400 MHz, CDCl₃) δ ppm 1.47 (s, 9H), 3.50 (br s, 1H), 3.58 (br s, 1H), 3.79 (dq, 1H, J=18.9 Hz, J=2.2 Hz), 3.97 (d, 1H, J=18.3 Hz), 4.19 (br s, 1H), 5.82 (d, 1H, J=9.9 Hz), 5.91 (dq, 1H, J=9.9 Hz, J=1.8 Hz).

### Step 3. Formation of tert-butyl 3-(pivaloyloxy)-3,6-dihydropyridine-1(2H)-carboxylate

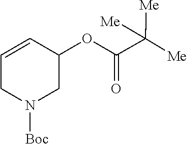 [SMILES: CC(C)(C)OC(=O)N1CC=CC(OC(=O)C(C)(C)C)C1]

tert-Butyl allyl(2-hydroxybut-3-en-1-yl)carbamate (5.3 g, 26.60 mmol, 1.0 equiv.) and DMAP (325 mg, 2.66 mmol, 0.1 equiv.) were dissolved in anhydrous DCM (89 mL, 0.3 M). Pyridine (10.7 mL, 133 mmol, 5.0 equiv.) and pivaloyl chloride (4.6 mL, 37.20 mmol, 1.4 equiv.) were added and the resulting mixture was stirred at room temperature overnight. The reaction mixture was diluted with water. The aqueous phase was extracted 3 times with DCM. The combined organic layers were washed with brine, dried over MgSO₄ and concentrated under vacuum. The crude was purified by chromatography on silica gel (hexanes:EtOAc 4:1), to obtain tert-butyl 3-(pivaloyloxy)-3,6-dihydropyridine-1(2H)-carboxylate as a colorless oil (7.0 g, 24.70 mmol, 93%). ¹H NMR (400 MHz, CDCl₃) δ ppm 1.19 (s, 9H), 1.46 (s, 9H), 3.46 (dd, 1H, J=13.7 Hz, J=3.5 Hz), 3.78 (dd, 2H, J=13.7 Hz, J=4.3 Hz), 4.02-4.26 (m, 2H), 5.17 (s, 1H), 5.83-5.97 (m, 3H).

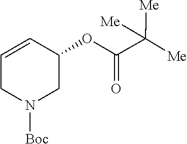 [SMILES: CC(C)(C)OC(=O)N1CC=C[C@H](OC(=O)C(C)(C)C)C1]

tert-Butyl (S)-3-hydroxy-3,6-dihydropyridine-1(2H)-carboxylate (1.4 g, 7.08 mmol, 1.0 equiv.) and DMAP (86 mg, 0.71 mmol, 0.1 equiv.) were dissolved in anhydrous DCM (23 mL, 0.3 M). Pyridine (2.8 mL, 35.40 mmol, 5.0 equiv.) and pivaloyl chloride (1.2 mL, 9.91 mmol, 1.4 equiv.) were added and the resulting mixture was stirred at room temperature overnight. The reaction mixture was diluted with water. The aqueous phase was extracted 3 times with DCM. The combined organic layers were washed with brine, dried over MgSO₄ and concentrated under vacuum. The crude was purified by chromatography on silica gel (hexanes:EtOAc 4:1), to obtain tert-butyl (S)-3-(pivaloyloxy)-3,6-dihydropyridine-1(2H)-carboxylate as a colorless oil (1.9 g, 6.74 mmol, 95%). ¹H NMR (400 MHz, CDCl₃) δ ppm 1.19 (s, 9H), 1.46 (s, 9H), 3.46 (dd, 1H, J=13.7 Hz, J=3.5 Hz), 3.78 (dd, 2H, J=13.7 Hz, J=4.3 Hz), 4.02-4.26 (m, 2H), 5.17 (s, 1H), 5.83-5.97 (m, 3H).

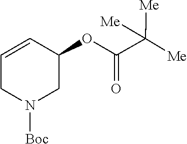 [SMILES: CC(C)(C)OC(=O)N1CC=C[C@@H](OC(=O)C(C)(C)C)C1]

tert-Butyl (R)-3-hydroxy-3,6-dihydropyridine-1(2H)-carboxylate (758 mg, 3.80 mmol, 1.0 equiv.) and DMAP (93 mg, 0.76 mmol, 0.2 equiv.) were dissolved in anhydrous DCM (13 mL, 0.3 M). Pyridine (1.5 mL, 19.02 mmol, 5.0 equiv.) and pivaloyl chloride (703 μL, 5.71 mmol, 1.4 equiv.) were added and the resulting mixture was stirred at room temperature overnight. The reaction mixture was diluted with water. The aqueous phase was extracted 3 times with DCM. The combined organic layers were washed with brine, dried over MgSO₄ and concentrated under vacuum. The crude was purified by chromatography on silica gel (hexanes:EtOAc 4:1), to obtain tert-butyl (R)-3-(pivaloyloxy)-3,6-dihydropyridine-1(2H)-carboxylate as a colorless oil (1.1 g, 3.74 mmol, 98%). ¹H NMR (400 MHz, CDCl₃) δ ppm 1.19 (s, 9H), 1.46 (s, 9H), 3.46 (dd, 1H, J=13.7 Hz, J=3.5 Hz), 3.78 (dd, 2H, J=13.7 Hz, J=4.3 Hz), 4.02-4.26 (m, 2H), 5.17 (s, 1H), 5.83-5.97 (m, 3H).

### Step 4. Formation of 1,2,3,6-tetrahydropyridin-3-yl pivalate

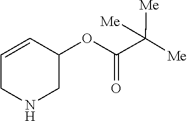 [SMILES: CC(C)(C)C(=O)OC1C=CCNC1]

Following general procedure B and starting from tert-butyl 3-(pivaloyloxy)-3,6-dihydropyridine-1(2H)-carboxylate (2.6 g, 9.07 mmol, 1.0 equiv.), 1,2,3,6-tetrahydropyridin-3-yl pivalate was obtained as a brown oil (1.3 g, 9.07 mmol, 76%).

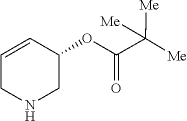 [SMILES: CC(C)(C)C(=O)O[C@H]1C=CCNC1]

Following general procedure B and starting from tert-butyl (S)-3-(pivaloyloxy)-3,6-dihydropyridine-1(2H)-carboxylate (4.5 g, 15.88 mmol, 1.0 equiv.), (S)-1,2,3,6-tetrahydropyridin-3-yl pivalate was obtained as a brown oil (2.2 g, 11.84 mmol, 75%).

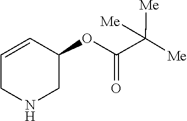 [SMILES: CC(C)(C)C(=O)O[C@@H]1C=CCNC1]

Following general procedure B and starting from tert-butyl (R)-3-(pivaloyloxy)-3,6-dihydropyridine-1(2H)-carboxylate (1.1 g, 3.71 mmol, 1.0 equiv.), (R)-1,2,3,6-tetrahydropyridin-3-yl pivalate was obtained as a brown oil (0.7 g, 3.55 mmol, 96%).

### Step 5. Reductive Amination

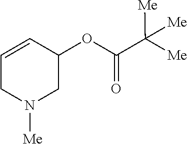 [SMILES: CN1CC=CC(OC(=O)C(C)(C)C)C1]

Following general procedure C and starting from 1,2,3,6-tetrahydropyridin-3-yl pivalate (540 mg, 2.95 mmol, 1.0 equiv.) and formaldehyde (37% in water, 658 μL, 8.84 mmol, 3.0 equiv.), 1-methyl-1,2,3,6-tetrahydropyridin-3-yl pivalate was obtained as an orange oil (381 mg, 1.93 mmol, 66%). ¹H NMR (400 MHz, CDCl₃) δ ppm 1.20 (s, 9H), 2.36 (s, 3H), 2.46 (dd, 1H, J=11.7 Hz, J=5.4 Hz), 2.79 (dd, 1H, J=11.7 Hz, J=4.9 Hz), 2.88 (d, 1H, J=16.7 Hz), 2.98 (dd, 1H, J=16.7 Hz, J=1.1 Hz), 5.31 (s, 1H), 5.73 (dd, 1H, J=10.1 Hz, J=1.5 Hz), 5.95 (d, 1H, J=10.1 Hz).

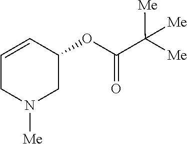 [SMILES: CN1CC=C[C@H](OC(=O)C(C)(C)C)C1]

Following general procedure C and starting from (S)-1,2,3,6-tetrahydropyridin-3-yl pivalate (272 mg, 1.48 mmol, 1.0 equiv.) and formaldehyde (37% in water, 332 μL, 4.45 mmol, 3.0 equiv.), (S)-1-methyl-1,2,3,6-tetrahydropyridin-3-yl pivalate was obtained as a colorless oil (193 mg, 0.96 mmol, 66%). ¹H NMR (400 MHz, CDCl₃) δ ppm 1.20 (s, 9H), 2.36 (s, 3H), 2.46 (dd, 1H, J=11.7 Hz, J=5.4 Hz), 2.79 (dd, 1H, J=11.7 Hz, J=4.9 Hz), 2.88 (d, 1H, J=16.7 Hz), 2.98 (dd, 1H, J=16.7 Hz, J=1.1 Hz), 5.31 (s, 1H), 5.73 (dd, 1H, J=10.1 Hz, J=1.5 Hz), 5.95 (d, 1H, J=10.1 Hz).

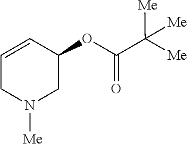 [SMILES: CN1CC=C[C@@H](OC(=O)C(C)(C)C)C1]

Following general procedure C and starting from (R)-1,2,3,6-tetrahydropyridin-3-yl pivalate (487 mg, 2.66 mmol, 1.0 equiv.) and formaldehyde (37% in water, 594 μL, 7.97 mmol, 3.0 equiv.), (R)-1-methyl-1,2,3,6-tetrahydropyridin-3-yl pivalate was obtained as a colorless oil (292 mg, 1.48 mmol, 56%). ¹H NMR (400 MHz, CDCl₃) δ ppm 1.20 (s, 9H), 2.36 (s, 3H), 2.46 (dd, 1H, J=11.7 Hz, J=5.4 Hz), 2.79 (dd, 1H, J=11.7 Hz, J=4.9 Hz), 2.88 (d, 1H, J=16.7 Hz), 2.98 (dd, 1H, J=16.7 Hz, J=1.1 Hz), 5.31 (s, 1H), 5.73 (dd, 1H, J=10.1 Hz, J=1.5 Hz), 5.95 (d, 1H, J=10.1 Hz).

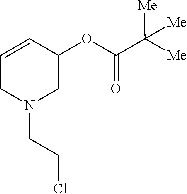 [SMILES: CC(C)(C)C(=O)OC1C=CCN(CCCl)C1]

Following general procedure C and starting from 1,2,3,6-tetrahydropyridin-3-yl pivalate (317 mg, 1.73 mmol, 1.0 equiv.) and 2-chloroacetaldehyde (45% in water, 732 μL, 5.19 mmol, 3.0 equiv.), 1-(2-chloroethyl)-1,2,3,6-tetrahydropyridin-3-yl pivalate was obtained as a colorless oil (54 mg, 0.22 mmol, 13%). ¹H NMR (400 MHz, CDCl₃) δ ppm 1.20 (s, 9H), 2.65 (dd, 1H, J=11.2 Hz, J=4.2 Hz), 2.79-2.92 (m, 3H), 3.07 (d, 1H, J=16.9 Hz), 3.15 (d, 1H, J=16.6 Hz), 3.59 (t, 1H, J=7.1 Hz), 5.27 (br s, 1H), 5.76 (dq, 1H, J=10.3 Hz, J=2.3 Hz), 5.94 (dt, 1H, J=10.1 Hz, J=3.1 Hz).

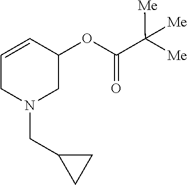 [SMILES: CC(C)(C)C(=O)OC1C=CCN(CC2CC2)C1]

Following general procedure D and starting from 1,2,3,6-tetrahydropyridin-3-yl pivalate (230 mg, 1.26 mmol, 1.0 equiv.) and cyclopropanecarbaldehyde (281 μL, 3.77 mmol, 3.0 equiv.), 1-(cyclopropylmethyl)-1,2,3,6-tetrahydropyridin-3-yl pivalate was obtained as a colorless oil (282 mg, 1.19 mmol, 95%). ¹H NMR (400 MHz, CDCl₃) δ ppm 0.12 (q, 2H, J=4.2 Hz), 0.5-0.52 (m, 2H), 0.87-0.91 (m, 1H), 1.20 (s, 9H), 2.26 (dd, 1H, J=12.7 Hz, J=6.9 Hz), 2.45 (dd, 1H, J=12.6 Hz, J=6.3 Hz), 2.61 (dd, 1H, J=11.7 Hz, J=5.6 Hz), 2.90 (dd, 1H, J=11.7 Hz, J=4.9 Hz), 3.07 (br s, 2H), 5.33 (br s, 1H), 5.73 (dq, 1H, J=10.1 Hz, J=2.7 Hz), 5.97 (dtd, 1H, J=9.9 Hz, J=3.2 Hz, J=3.2 Hz); ¹³C NMR (100 MHz, CDCl₃) δ ppm 3.8, 3.9, 4.6, 8.6, 27.5, 39.1, 52.5, 54.5, 63.1, 67.6, 124.8, 130.7, 178.7.

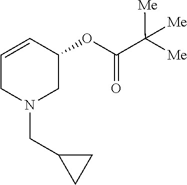 [SMILES: CC(C)(C)C(=O)O[C@H]1C=CCN(CC2CC2)C1]

Following general procedure D and starting from (S)-1,2,3,6-tetrahydropyridin-3-yl pivalate (136 mg, 0.74 mmol, 1.0 equiv.) and cyclopropanecarbaldehyde (166 μL, 3.77 mmol, 3.0 equiv.), (S)-1-(cyclopropylmethyl)-1,2,3,6-tetrahydropyridin-3-yl pivalate was obtained as a colorless oil (151 mg, 0.64 mmol, 86%). ¹H NMR (400 MHz, CDCl₃) δ ppm 0.12 (q, 2H, J=4.2 Hz), 0.5-0.52 (m, 2H), 0.87-0.91 (m, 1H), 1.20 (s, 9H), 2.26 (dd, 1H, J=12.7 Hz, J=6.9 Hz), 2.45 (dd, 1H, J=12.6 Hz, J=6.3 Hz), 2.61 (dd, 1H, J=11.7 Hz, J=5.6 Hz), 2.90 (dd, 1H, J=11.7 Hz, J=4.9 Hz), 3.07 (br s, 2H), 5.33 (br s, 1H), 5.73 (dq, 1H, J=10.1 Hz, J=2.7 Hz), 5.97 (dtd, 1H, J=9.9 Hz, J=3.2 Hz, J=3.2 Hz); ¹³C NMR (100 MHz, CDCl₃) δ ppm 3.8, 3.9, 4.6, 8.6, 27.5, 39.1, 52.5, 54.5, 63.1, 67.6, 124.8, 130.7, 178.7.

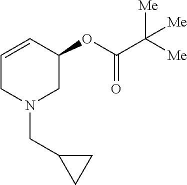 [SMILES: CC(C)(C)C(=O)O[C@@H]1C=CCN(CC2CC2)C1]

Following general procedure D and starting from (R)-1,2,3,6-tetrahydropyridin-3-yl pivalate (192 mg, 1.05 mmol, 1.0 equiv.) and cyclopropanecarbaldehyde (235 μL, 3.14 mmol, 3.0 equiv.), (R)-1-(cyclopropylmethyl)-1,2,3,6-tetrahydropyridin-3-yl pivalate was obtained as a colorless oil (249 mg, 1.05 mmol, 100%). ¹H NMR (400 MHz, CDCl₃) δ ppm 0.12 (q, 2H, J=4.2 Hz), 0.5-0.52 (m, 2H), 0.87-0.91 (m, 1H), 1.20 (s, 9H), 2.26 (dd, 1H, J=12.7 Hz, J=6.9 Hz), 2.45 (dd, 1H, J=12.6 Hz, J=6.3 Hz), 2.61 (dd, 1H, J=11.7 Hz, J=5.6 Hz), 2.90 (dd, 1H, J=11.7 Hz, J=4.9 Hz), 3.07 (br s, 2H), 5.33 (br s, 1H), 5.73 (dq, 1H, J=10.1 Hz, J=2.7 Hz), 5.97 (dtd, 1H, J=9.9 Hz, J=3.2 Hz, J=3.2 Hz); ¹³C NMR (100 MHz, CDCl₃) δ ppm 3.8, 3.9, 4.6, 8.6, 27.5, 39.1, 52.5, 54.5, 63.1, 67.6, 124.8, 130.7, 178.7.

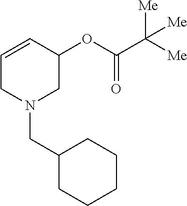 [SMILES: CC(C)(C)C(=O)OC1C=CCN(CC2CCCCC2)C1]

Following general procedure C and starting from 1,2,3,6-tetrahydropyridin-3-yl pivalate (122 mg, 0.67 mmol, 1.0 equiv.) and cyclohexanecarbaldehyde (242 μL, 2.00 mmol, 3.0 equiv.), 1-(cyclohexylmethyl)-1,2,3,6-tetrahydropyridin-3-yl pivalate was obtained as a colorless oil (110 mg, 0.39 mmol, 59%). ¹H NMR (400 MHz, CDCl₃) δ ppm 0.86 (q, 2H, J=11.7 Hz), 1.15-1.22 (m, 2H), 1.20 (s, 9H), 1.50 (br s, 1H), 1.66-1.76 (m, 5H), 1.85 (d, 1H, J=13.5 Hz), 2.16-2.24 (m, 2H), 2.57-2.64 (m, 2H), 2.87 (d, 1H, J=16.7 Hz), 3.02 (d, 1H, J=16.4 Hz), 5.25 (br s, 1H), 5.73 (d, 1H, J=9.9 Hz), 5.95 (dt, 1H, J=9.9 Hz, J=3.1 Hz).

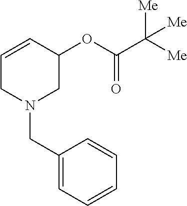 [SMILES: CC(C)(C)C(=O)OC1C=CCN(Cc2ccccc2)C1]

Following general procedure D and starting from 1,2,3,6-tetrahydropyridin-3-yl pivalate (180 mg, 0.98 mmol, 1.0 equiv.) and benzaldehyde (300 μL, 2.95 mmol, 3.0 equiv.), 1-benzyl-1,2,3,6-tetrahydropyridin-3-yl pivalate was obtained as a colorless oil (198 mg, 0.72 mmol, 74%). ¹H NMR (400 MHz, CDCl₃) δ ppm 1.18 (s, 9H), 2.61 (dd, 1H, J=11.9 Hz, J=4.7 Hz), 2.70 (dd, 1H, J=11.9 Hz, J=4.5 Hz), 2.94 (d, 1H, J=16.8 Hz), 3.11 (d, 1H, J=16.5 Hz), 3.51 (d, 1H, J=13.4 Hz), 3.73 (d, 1H, J=13.4 Hz), 5.26 (br s, 1H), 5.76 (dq, 1H, J=9.9 Hz, J=2.9 Hz), 5.97 (dt, 1H, J=9.9 Hz, J=3.1 Hz), 7.24 (t, 1H, J=7.0 Hz), 7.31 (t, 2H, J=7.1 Hz), 7.36 (d, 2H, J=7.3 Hz).

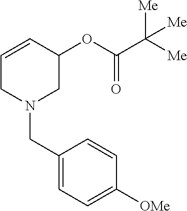 [SMILES: COc1ccc(CN2CC=CC(OC(=O)C(C)(C)C)C2)cc1]

Following general procedure D and starting from 1,2,3,6-tetrahydropyridin-3-yl pivalate (250 mg, 1.36 mmol, 1.0 equiv.) and 4-methoxybenzaldehyde (498 μL, 4.09 mmol, 3.0 equiv.), 1-(4-methoxybenzyl)-1,2,3,6-tetrahydropyridin-3-yl pivalate was obtained as a colorless oil (414 mg, 1.36 mmol, 100%). ¹H NMR (400 MHz, CDCl₃) δ ppm 1.19 (s, 9H), 2.56-2.71 (m, 2H), 2.89-2.96 (m, 1H), 3.06-3.14 (m, 1H), 3.44-3.48 (m, 1H), 3.61-3.68 (m, 1H), 3.79 (s, 3H), 5.76 (d, 1H, J=10.0 Hz), 5.96 (dtd, 1H, J=10.0 Hz, J=3.3 Hz, J=1.2 Hz), 6.84 (d, 2H, J=8.6 Hz), 7.00 (d, 2H, J=8.6 Hz).

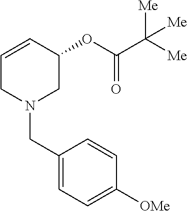 [SMILES: COc1ccc(CN2CC=C[C@H](OC(=O)C(C)(C)C)C2)cc1]

Following general procedure D and starting from (S)-1,2,3,6-tetrahydropyridin-3-yl pivalate (177 mg, 0.97 mmol, 1.0 equiv.) and 4-methoxybenzaldehyde (353 μL, 2.90 mmol, 3.0 equiv.), (S)-1-(4-methoxybenzyl)-1,2,3,6-tetrahydropyridin-3-yl pivalate was obtained as a colorless oil (251 mg, 0.83 mmol, 86%). ¹H NMR (400 MHz, CDCl₃) δ ppm 1.19 (s, 9H), 2.56-2.71 (m, 2H), 2.89-2.96 (m, 1H), 3.06-3.14 (m, 1H), 3.44-3.48 (m, 1H), 3.61-3.68 (m, 1H), 3.79 (s, 3H), 5.76 (d, 1H, J=10.0 Hz), 5.96 (dtd, 1H, J=10.0 Hz, J=3.3 Hz, J=1.2 Hz), 6.84 (d, 2H, J=8.6 Hz), 7.00 (d, 2H, J=8.6 Hz).

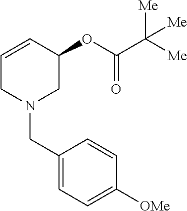 [SMILES: COc1ccc(CN2CC=C[C@@H](OC(=O)C(C)(C)C)C2)cc1]

Following general procedure D and starting from (R)-1,2,3,6-tetrahydropyridin-3-yl pivalate (162 mg, 0.88 mmol, 1.0 equiv.) and 4-methoxybenzaldehyde (323 μL, 2.65 mmol, 3.0 equiv.), (R)-1-(4-methoxybenzyl)-1,2,3,6-tetrahydropyridin-3-yl pivalate was obtained as a colorless oil (268 mg, 0.88 mmol, 100%). ¹H NMR (400 MHz, CDCl₃) δ ppm 1.19 (s, 9H), 2.56-2.71 (m, 2H), 2.89-2.96 (m, 1H), 3.06-3.14 (m, 1H), 3.44-3.48 (m, 1H), 3.61-3.68 (m, 1H), 3.79 (s, 3H), 5.76 (d, 1H, J=10.0 Hz), 5.96 (dtd, 1H, J=10.0 Hz, J=3.3 Hz, J=1.2 Hz), 6.84 (d, 2H, J=8.6 Hz), 7.00 (d, 2H, J=8.6 Hz).

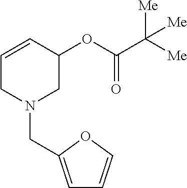 [SMILES: CC(C)(C)C(=O)OC1C=CCN(Cc2ccco2)C1]

Following general procedure D and starting from 1,2,3,6-tetrahydropyridin-3-yl pivalate (323 mg, 1.76 mmol, 1.0 equiv.) and furan-2-carbaldehyde (438 μL, 5.29 mmol, 3.0 equiv.), 1-(furan-2-ylmethyl)-1,2,3,6-tetrahydropyridin-3-yl pivalate was obtained as an orange oil (314 mg, 1.19 mmol, 68%). ¹H NMR (400 MHz, CDCl₃) δ ppm 1.18 (s, 9H), 2.56 (dd, 1H, J=11.8 Hz, J=5.4 Hz), 2.85 (dd, 1H, J=11.8 Hz, J=4.8 Hz), 3.00 (d, 1H, J=16.9 Hz), 3.10 (d, 1H, J=16.7 Hz), 3.61 (d, 1H, J=14.2 Hz), 3.73 (d, 1H, J=14.2 Hz), 5.30 (br s, 1H), 5.72 (dq, 1H, J=9.9 Hz, J=2.6 Hz), 5.93 (dt, 1H, J=9.9 Hz, J=3.4 Hz), 6.22 (d, 1H, J=3.2 Hz), 6.31 (dd, 1H, J=3.1 Hz, J=1.8 Hz), 7.36 (d, 1H, J=1.8 Hz).

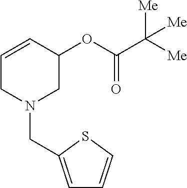 [SMILES: CC(C)(C)C(=O)OC1C=CCN(Cc2cccs2)C1]

Following general procedure D and starting from 1,2,3,6-tetrahydropyridin-3-yl pivalate (230 mg, 1.26 mmol, 1.0 equiv.) and thiophene-2-carbaldehyde (352 μL, 3.77 mmol, 3.0 equiv.), 1-(thiophen-2-ylmethyl)-1,2,3,6-tetrahydropyridin-3-yl pivalate was obtained as a yellow oil (351 mg, 1.26 mmol, 100%). ¹H NMR (400 MHz, CDCl₃) δ ppm 1.19 (s, 9H), 2.63 (dd, 1H, J=11.8 Hz, J=4.9 Hz), 2.78 (dd, 1H, J=12.4 Hz, J=4.8 Hz), 3.00 (d, 1H, J=16.6 Hz), 3.15 (d, 1H, J=17.0 Hz), 3.77 (d, 1H, J=14.0 Hz), 3.90 (d, 1H, J=14.0 Hz), 5.28 (br s, 1H), 5.75 (dq, 1H, J=9.9 Hz, J=2.5 Hz), 5.95 (dt, 1H, J=10.1 Hz, J=3.2 Hz), 6.92-6.95 (m, 2H), 7.21-7.23 (m, 1H).

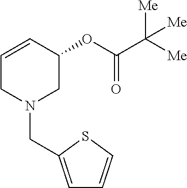 [SMILES: CC(C)(C)C(=O)O[C@H]1C=CCN(Cc2cccs2)C1]

Following general procedure D and starting from (S)-1,2,3,6-tetrahydropyridin-3-yl pivalate (136 mg, 0.74 mmol, 1.0 equiv.) and thiophene-2-carbaldehyde (208 μL, 2.23 mmol, 3.0 equiv.), (S)-1-(thiophen-2-ylmethyl)-1,2,3,6-tetrahydropyridin-3-yl pivalate was obtained as a yellow oil (182 mg, 0.65 mmol, 88%). ¹H NMR (400 MHz, CDCl₃) δ ppm 1.19 (s, 9H), 2.63 (dd, 1H, J=11.8 Hz, J=4.9 Hz), 2.78 (dd, 1H, J=12.4 Hz, J=4.8 Hz), 3.00 (d, 1H, J=16.6 Hz), 3.15 (d, 1H, J=17.0 Hz), 3.77 (d, 1H, J=14.0 Hz), 3.90 (d, 1H, J=14.0 Hz), 5.28 (br s, 1H), 5.75 (dq, 1H, J=9.9 Hz, J=2.5 Hz), 5.95 (dt, 1H, J=10.1 Hz, J=3.2 Hz), 6.92-6.95 (m, 2H), 7.21-7.23 (m, 1H).

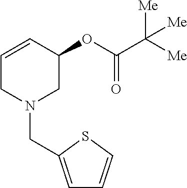 [SMILES: CC(C)(C)C(=O)O[C@@H]1C=CCN(Cc2cccs2)C1]

Following general procedure D and starting from (R)-1,2,3,6-tetrahydropyridin-3-yl pivalate (192 mg, 1.05 mmol, 1.0 equiv.) and thiophene-2-carbaldehyde (235 μL, 3.14 mmol, 3.0 equiv.), (R)-1-(thiophen-2-ylmethyl)-1,2,3,6-tetrahydropyridin-3-yl pivalate was obtained as a yellow oil (184 mg, 0.78 mmol, 74%). ¹H NMR (400 MHz, CDCl₃) δ ppm 1.19 (s, 9H), 2.63 (dd, 1H, J=11.8 Hz, J=4.9 Hz), 2.78 (dd, 1H, J=12.4 Hz, J=4.8 Hz), 3.00 (d, 1H, J=16.6 Hz), 3.15 (d, 1H, J=17.0 Hz), 3.77 (d, 1H, J=14.0 Hz), 3.90 (d, 1H, J=14.0 Hz), 5.28 (br s, 1H), 5.75 (dq, 1H, J=9.9 Hz, J=2.5 Hz), 5.95 (dt, 1H, J=10.1 Hz, J=3.2 Hz), 6.92-6.95 (m, 2H), 7.21-7.23 (m, 1H).

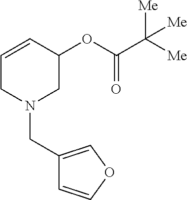 [SMILES: CC(C)(C)C(=O)OC1C=CCN(Cc2ccoc2)C1]

Following general procedure D and starting from 1,2,3,6-tetrahydropyridin-3-yl pivalate (230 mg, 1.26 mmol, 1.0 equiv.) and furan-3-carbaldehyde (326 μL, 3.77 mmol, 3.0 equiv.), 1-(furan-3-ylmethyl)-1,2,3,6-tetrahydropyridin-3-yl pivalate was obtained as a colorless oil (227 mg, 0.86 mmol, 68%). ¹H NMR (400 MHz, CDCl₃) δ ppm 1.18 (s, 9H), 2.57 (dd, 1H, J=11.7 Hz, J=4.9 Hz), 2.73 (dd, 1H, J=11.7 Hz, J=4.7 Hz), 2.94 (d, 1H, J=16.7 Hz), 3.08 (d, 1H, J=16.6 Hz), 3.41 (d, 1H, J=13.6 Hz), 3.56 (d, 1H, J=13.5 Hz), 5.27 (br s, 1H), 5.74 (dq, 1H, J=9.9 Hz, J=2.5 Hz), 5.96 (dt, 1H, J=9.9 Hz, J=2.5 Hz), 6.40 (s, 1H), 7.34-7.37 (m, 2H); ¹³C NMR (100 MHz, CDCl₃) δ ppm 27.5, 39.1, 52.4, 52.5, 53.9, 67.6, 111.5, 121.8, 124.7, 130.7, 141.1, 143.4, 178.6.

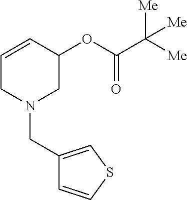 [SMILES: CC(C)(C)C(=O)OC1C=CCN(Cc2ccsc2)C1]

Following general procedure D and starting from 1,2,3,6-tetrahydropyridin-3-yl pivalate (323 mg, 1.76 mmol, 1.0 equiv.) and thiophene-3-carbaldehyde (463 μL, 5.29 mmol, 3.0 equiv.), 1-(thiophen-3-ylmethyl)-1,2,3,6-tetrahydropyridin-3-yl pivalate was obtained as a yellow oil (328 mg, 1.17 mmol, 67%). ¹H NMR (400 MHz, CDCl₃) δ ppm 1.18 (s, 9H), 2.59 (dd, 1H, J=11.7 Hz, J=4.7 Hz), 2.72 (dd, 1H, J=11.8 Hz, J=4.3 Hz), 2.94 (d, 1H, J=16.7 Hz), 3.10 (d, 1H, J=16.7 Hz), 3.56 (d, 1H, J=13.5 Hz), 3.71 (d, 1H, J=13.5 Hz), 5.27 (br s, 1H), 5.75 (d, 1H, J=9.8 Hz), 5.96 (d, 1H, J=9.9 Hz), 7.07 (d, 1H, J=4.8 Hz), 7.15 (s, 1H), 7.55 (d, 1H, J=5.0 Hz).

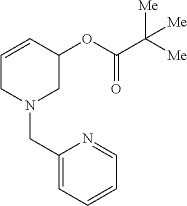 [SMILES: CC(C)(C)C(=O)OC1C=CCN(Cc2ccccn2)C1]

Following general procedure D and starting from 1,2,3,6-tetrahydropyridin-3-yl pivalate (230 mg, 1.26 mmol, 1.0 equiv.) and picolinaldehyde (358 μL, 3.77 mmol, 3.0 equiv.), 1-(pyridin-2-ylmethyl)-1,2,3,6-tetrahydropyridin-3-yl pivalate was obtained as a yellow oil (142 mg, 0.52 mmol, 41%). ¹H NMR (400 MHz, CDCl₃) δ ppm 1.19 (s, 9H), 2.63 (dd, 1H, J=11.9 Hz, J=4.9 Hz), 2.80 (dd, 1H, J=11.9 Hz, J=4.5 Hz), 3.04 (d, 1H, J=16.8 Hz), 3.17 (d, 1H, J=16.8 Hz), 3.70 (d, 1H, J=14.4 Hz), 3.85 (d, 1H, J=14.4 Hz), 4.02 (s, 2H), 5.28 (br s, 1H), 5.77 (dq, 1H, J=9.9 Hz, J=3.1 Hz), 5.97 (dt, 1H, J=10.1 Hz, J=2.7 Hz), 7.16 (dd, 1H, J=6.7 Hz, J=5.7 Hz), 7.53 (d, 1H, J=7.8 Hz), 7.64 (td, 1H, J=7.8 Hz, J=1.5 Hz), 8.53 (d, 1H, J=4.5 Hz); ¹³C NMR (100 MHz, CDCl₃) δ ppm 27.5, 39.1, 52.8, 54.3, 63.7, 67.5, 122.4, 123.1, 124.6, 130.8, 136.8, 149.4, 159.0.

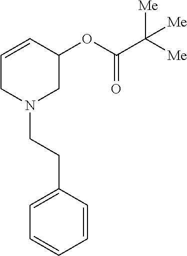 [SMILES: CC(C)(C)C(=O)OC1C=CCN(CCc2ccccc2)C1]

Following general procedure D and starting from 1,2,3,6-tetrahydropyridin-3-yl pivalate (317 mg, 1.73 mmol, 1.0 equiv.) and 2-phenylacetaldehyde (607 μL, 5.19 mmol, 3.0 equiv.), 1-phenethyl-1,2,3,6-tetrahydropyridin-3-yl pivalate was obtained as a colorless oil (304 mg, 1.06 mmol, 61%). ¹H NMR (400 MHz, CDCl₃) δ ppm 1.21 (s, 9H), 2.04-2.84 (m, 6H), 3.07-3.15 (m, 2H), 5.34 (br s, 1H), 5.74 (d, 1H, J=9.9 Hz), 5.97 (dt, 1H, J=9.9 Hz, J=2.9 Hz), 7.22 (d, 2H, J=7.5 Hz), 7.29 (t, 3H, J=7.2 Hz).

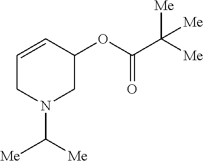 [SMILES: CC(C)N1CC=CC(OC(=O)C(C)(C)C)C1]

Following general procedure C and starting from 1,2,3,6-tetrahydropyridin-3-yl pivalate (230 mg, 1.26 mmol, 1.0 equiv.) and acetone (461 μL, 6.28 mmol, 5.0 equiv.), 1-isopropyl-1,2,3,6-tetrahydropyridin-3-yl pivalate was obtained as a colorless oil (219 mg, 0.97 mmol, 77%). ¹H NMR (400 MHz, CDCl₃) δ ppm 1.03 (d, 3H, J=6.5 Hz), 1.06 (d, 3H, J=6.6 Hz), 1.20 (s, 9H), 2.50 (dd, 1H, J=11.4 Hz, J=5.8 Hz), 2.79-2.87 (m, 2H), 3.04 (dd, 1H, J=16.6 Hz, J=1.9 Hz), 3.11 (dq, 1H, J=16.8 Hz, J=2.5 Hz), 5.29 (br s, 1H), 5.70 (dq, 1H, J=9.9 Hz, J=2.5 Hz), 5.98 (dt, 1H, J=9.9 Hz, J=3.5 Hz); ¹³C NMR (100 MHz, CDCl₃) δ ppm 17.9, 19.0, 27.5, 29.7, 48.7, 49.6, 53.9, 68.4, 124.9, 131.3, 178.8.

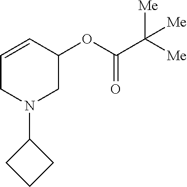 [SMILES: CC(C)(C)C(=O)OC1C=CCN(C2CCC2)C1]

Following general procedure C and starting from 1,2,3,6-tetrahydropyridin-3-yl pivalate (323 mg, 1.76 mmol, 1.0 equiv.) and cyclobutanone (395 μL, 5.29 mmol, 3.0 equiv.), 1-cyclobutyl-1,2,3,6-tetrahydropyridin-3-yl pivalate was obtained as a colorless oil (186 mg, 0.78 mmol, 44%). ¹H NMR (400 MHz, CDCl₃) δ ppm 1.20 (s, 9H), 1.65-1.74 (m, 2H), 1.90-2.04 (m, 4H), 2.30-2.37 (m, 3H), 2.61-2.68 (m, 2H), 5.31 (br s, 1H), 5.72 (dq, 1H, J=10.1 Hz, J=2.2 Hz), 5.95 (dt, 1H, J=9.9 Hz, J=3.1 Hz); ¹³C NMR (100 MHz, CDCl₃) δ ppm 13.2, 14.7, 27.2, 27.5, 36.8, 48.7, 50.5, 59.6, 67.2, 125.0, 130.1, 178.7.

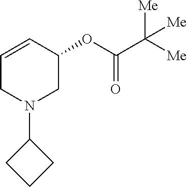 [SMILES: CC(C)(C)C(=O)O[C@H]1C=CCN(C2CCC2)C1]

Following general procedure C and starting from (S)-1,2,3,6-tetrahydropyridin-3-yl pivalate (136 mg, 0.74 mmol, 1.0 equiv.) and cyclobutanone (166 μL, 2.23 mmol, 3.0 equiv.), (S)-1-cyclobutyl-1,2,3,6-tetrahydropyridin-3-yl pivalate was obtained as a colorless oil (84 mg, 0.36 mmol, 48%). ¹H NMR (400 MHz, CDCl₃) δ ppm 1.20 (s, 9H), 1.65-1.74 (m, 2H), 1.90-2.04 (m, 4H), 2.30-2.37 (m, 3H), 2.61-2.68 (m, 2H), 5.31 (br s, 1H), 5.72 (dq, 1H, J=10.1 Hz, J=2.2 Hz), 5.95 (dt, 1H, J=9.9 Hz, J=3.1 Hz); ¹³C NMR (100 MHz, CDCl₃) δ ppm 13.2, 14.7, 27.2, 27.5, 36.8, 48.7, 50.5, 59.6, 67.2, 125.0, 130.1, 178.7.

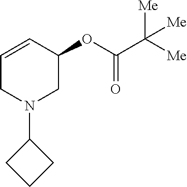 [SMILES: CC(C)(C)C(=O)O[C@@H]1C=CCN(C2CCC2)C1]

Following general procedure C and starting from (R)-1,2,3,6-tetrahydropyridin-3-yl pivalate (192 mg, 1.05 mmol, 1.0 equiv.) and cyclobutanone (235 μL, 3.14 mmol, 3.0 equiv.), (R)-1-cyclobutyl-1,2,3,6-tetrahydropyridin-3-yl pivalate was obtained as a yellow oil (184 mg, 0.78 mmol, 74%). ¹H NMR (400 MHz, CDCl₃) δ ppm 1.20 (s, 9H), 1.65-1.74 (m, 2H), 1.90-2.04 (m, 4H), 2.30-2.37 (m, 3H), 2.61-2.68 (m, 2H), 5.31 (br s, 1H), 5.72 (dq, 1H, J=10.1 Hz, J=2.2 Hz), 5.95 (dt, 1H, J=9.9 Hz, J=3.1 Hz); ¹³C NMR (100 MHz, CDCl₃) δ ppm 13.2, 14.7, 27.2, 27.5, 36.8, 48.7, 50.5, 59.6, 67.2, 125.0, 130.1, 178.7.

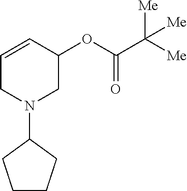 [SMILES: CC(C)(C)C(=O)OC1C=CCN(C2CCCC2)C1]

Following general procedure C and starting from 1,2,3,6-tetrahydropyridin-3-yl pivalate (275 mg, 1.50 mmol, 1.0 equiv.) and cyclopentanone (398 μL, 4.50 mmol, 3.0 equiv.), 1-cyclopentyl-1,2,3,6-tetrahydropyridin-3-yl pivalate was obtained as a colorless oil (240 mg, 0.96 mmol, 64%). ¹H NMR (400 MHz, CDCl₃) δ ppm 1.20 (s, 9H), 1.38-1.58 (m, 4H), 1.64-1.70 (m, 2H), 1.81-1.90 (m, 2H), 2.02-2.06 (m, 1H), 2.49 (dd, 1H, J=11.7 Hz, J=5.9 Hz), 2.67 (quint., 1H, J=7.8 Hz), 2.88 (dd, 1H, J=11.7 Hz, J=5.0 Hz), 3.04 (br s, 1H), 5.32 (br s, 1H), 5.71 (dq, 1H, J=9.9 Hz, J=2.2 Hz), 5.95 (dt, 1H, J=9.9 Hz, J=3.3 Hz).

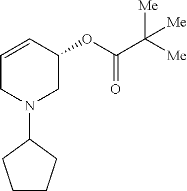 [SMILES: CC(C)(C)C(=O)O[C@H]1C=CCN(C2CCCC2)C1]

Following general procedure C and starting from (S)-1,2,3,6-tetrahydropyridin-3-yl pivalate (136 mg, 0.74 mmol, 1.0 equiv.) and cyclopentanone (197 μL, 2.23 mmol, 3.0 equiv.), (S)-1-cyclopentyl-1,2,3,6-tetrahydropyridin-3-yl pivalate was obtained as a colorless oil (99 mg, 0.39 mmol, 53%). ¹H NMR (400 MHz, CDCl₃) δ ppm 1.20 (s, 9H), 1.38-1.58 (m, 4H), 1.64-1.70 (m, 2H), 1.81-1.90 (m, 2H), 2.02-2.06 (m, 1H), 2.49 (dd, 1H, J=11.7 Hz, J=5.9 Hz), 2.67 (quint., 1H, J=7.8 Hz), 2.88 (dd, 1H, J=11.7 Hz, J=5.0 Hz), 3.04 (br s, 1H), 5.32 (br s, 1H), 5.71 (dq, 1H, J=9.9 Hz, J=2.2 Hz), 5.95 (dt, 1H, J=9.9 Hz, J=3.3 Hz).

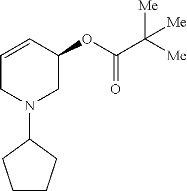 [SMILES: CC(C)(C)C(=O)O[C@@H]1C=CCN(C2CCCC2)C1]

Following general procedure C and starting from (R)-1,2,3,6-tetrahydropyridin-3-yl pivalate (192 mg, 1.05 mmol, 1.0 equiv.) and cyclopentanone (278 μL, 3.14 mmol, 3.0 equiv.), (R)-1-cyclopentyl-1,2,3,6-tetrahydropyridin-3-yl pivalate was obtained as a colorless oil (194 mg, 0.77 mmol, 74%). ¹H NMR (400 MHz, CDCl₃) δ ppm 1.20 (s, 9H), 1.38-1.58 (m, 4H), 1.64-1.70 (m, 2H), 1.81-1.90 (m, 2H), 2.02-2.06 (m, 1H), 2.49 (dd, 1H, J=11.7 Hz, J=5.9 Hz), 2.67 (quint., 1H, J=7.8 Hz), 2.88 (dd, 1H, J=11.7 Hz, J=5.0 Hz), 3.04 (br s, 1H), 5.32 (br s, 1H), 5.71 (dq, 1H, J=9.9 Hz, J=2.2 Hz), 5.95 (dt, 1H, J=9.9 Hz, J=3.3 Hz).

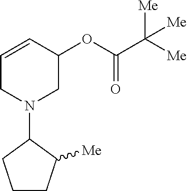 [SMILES: CC1CCCC1N1CC=CC(OC(=O)C(C)(C)C)C1]

Following general procedure C and starting from 1,2,3,6-tetrahydropyridin-3-yl pivalate (188 mg, 1.03 mmol, 1.0 equiv.) and 2-methylcyclopentan-1-one (329 μL, 3.08 mmol, 3.0 equiv.), 1-(2-methylcyclopentyl)-1,2,3,6-tetrahydropyridin-3-yl pivalate was obtained as a colorless oil (152 mg, 0.57 mmol, 56%). ¹H NMR (400 MHz, CDCl₃) δ ppm 0.84 (d, 3H, J=6.9 Hz), 1.20 (s, 9H), 1.40-1.43 (m, 1H), 1.55-1.60 (m, 2H), 1.70-1.78 (m, 3H), 2.15-2.38 (m, 2H), 2.18 (dd, 1H, J=11.9 Hz, J=2.9 Hz), 2.71-3.22 (m, 3H), 5.21 (br s, 0.5H), 5.32 (br s, 0.5H), 5.70 (d, 0.5H, J=9.1 Hz), 5.77 (d, 0.5H, J=9.3 Hz), 5.91 (d, 0.5H, J=9.9 Hz), 5.99 (d, 0.5H, J=9.5 Hz).

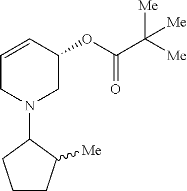 [SMILES: CC1CCCC1N1CC=C[C@H](OC(=O)C(C)(C)C)C1]

Following general procedure C and starting from (S)-1,2,3,6-tetrahydropyridin-3-yl pivalate (592 mg, 3.23 mmol, 1.0 equiv.) and 2-methylcyclopentan-1-one (1.0 mL, 9.69 mmol, 3.0 equiv.), (S)-1-(2-methylcyclopentyl)-1,2,3,6-tetrahydropyridin-3-yl pivalate was obtained as a colorless oil (215 mg, 0.81 mmol, 25%). ¹H NMR (400 MHz, CDCl₃) δ ppm 0.84 (d, 3H, J=6.9 Hz), 1.20 (s, 9H), 1.40-1.43 (m, 1H), 1.55-1.60 (m, 2H), 1.70-1.78 (m, 3H), 2.15-2.38 (m, 2H), 2.18 (dd, 1H, J=11.9 Hz, J=2.9 Hz), 2.71-3.22 (m, 3H), 5.21 (br s, 0.5H), 5.32 (br s, 0.5H), 5.70 (d, 0.5H, J=9.1 Hz), 5.77 (d, 0.5H, J=9.3 Hz), 5.91 (d, 0.5H, J=9.9 Hz), 5.99 (d, 0.5H, J=9.5 Hz).

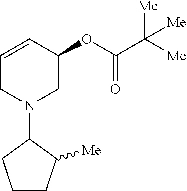 [SMILES: CC1CCCC1N1CC=C[C@@H](OC(=O)C(C)(C)C)C1]

Following general procedure C and starting from (R)-1,2,3,6-tetrahydropyridin-3-yl pivalate (340 mg, 186 mmol, 1.0 equiv.) and 2-methylcyclopentan-1-one (596 μL, 1.86 mmol, 3.0 equiv.), (R)-1-(2-methylcyclopentyl)-1,2,3,6-tetrahydropyridin-3-yl pivalate was obtained as a colorless oil (287 mg, 1.08 mmol, 58%). ¹H NMR (400 MHz, CDCl₃) δ ppm 0.84 (d, 3H, J=6.9 Hz), 1.20 (s, 9H), 1.40-1.43 (m, 1H), 1.55-1.60 (m, 2H), 1.70-1.78 (m, 3H), 2.15-2.38 (m, 2H), 2.18 (dd, 1H, J=11.9 Hz, J=2.9 Hz), 2.71-3.22 (m, 3H), 5.21 (br s, 0.5H), 5.32 (br s, 0.5H), 5.70 (d, 0.5H, J=9.1 Hz), 5.77 (d, 0.5H, J=9.3 Hz), 5.91 (d, 0.5H, J=9.9 Hz), 5.99 (d, 0.5H, J=9.5 Hz).

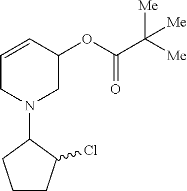 [SMILES: CC(C)(C)C(=O)OC1C=CCN(C2CCCC2Cl)C1]

Following general procedure D and starting from 1,2,3,6-tetrahydropyridin-3-yl pivalate (236 mg, 1.29 mmol, 1.0 equiv.) and 2-chlorocyclopentan-1-one (387 μL, 3.86 mmol, 3.0 equiv.), 1-(2-chlorocyclopentyl)-1,2,3,6-tetrahydropyridin-3-yl pivalate was obtained as a colorless oil (121 mg, 0.42 mmol, 33%). ¹H NMR (400 MHz, CDCl₃) δ ppm 1.20 (s, 9H), 1.69-1.97 (m, 4H), 2.08-2.11 (m, 2H), 2.61 (br s, 1H), 2.81 (br s, 1H), 3.07-3.24 m, 3H), 4.44 (s, 1H), 5.33 (br s, 1H), 5.78 (d, 1H, J=9.4 Hz), 5.92 (d, 1H, J=9.4 Hz).

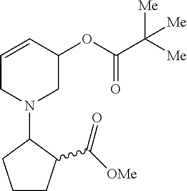 [SMILES: COC(=O)C1CCCC1N1CC=CC(OC(=O)C(C)(C)C)C1]

Following general procedure C and starting from 1,2,3,6-tetrahydropyridin-3-yl pivalate (340 mg, 1.86 mmol, 1.0 equiv.) and methyl 2-oxocyclopentane-1-carboxylate (691 μL, 5.57 mmol, 3.0 equiv.), methyl 2-(3-(pivaloyloxy)-3,6-dihydropyridin-1(2H)-yl)cyclopentane-1-carboxylate was obtained as a colorless oil (177 mg, 0.57 mmol, 31%). ¹H NMR (400 MHz, CDCl₃) δ ppm 1.20 (s, 9H), 1.60 (br s, 2H), 1.78-1.82 (m, 3H), 1.95-1.99 (m, 2H), 2.37 (br s, 1H), 2.80 (br s, 1H), 3.05-3.17 (m, 3H), 3.69 (s, 3H), 5.22 (br s, 1H), 5.66 (d, 1H, J=9.3 Hz), 5.86 (d, 1H, J=9.3 Hz).

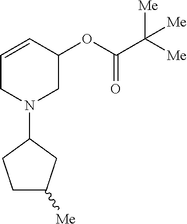 [SMILES: CC1CCC(N2CC=CC(OC(=O)C(C)(C)C)C2)C1]

Following general procedure C and starting from 1,2,3,6-tetrahydropyridin-3-yl pivalate (188 mg, 1.03 mmol, 1.0 equiv.) and 3-methylcyclopentan-1-one (331 μL, 3.08 mmol, 3.0 equiv.), 1-(3-methylcyclopentyl)-1,2,3,6-tetrahydropyridin-3-yl pivalate was obtained as a colorless oil (201 mg, 0.76 mmol, 74%). ¹H NMR (400 MHz, CDCl₃) δ ppm 0.96 (d, 1H, J=6.7 Hz), 1.00 (d, 2H, J=6.5 Hz), 1.20 (s, 9H), 1.35-1.55 (m, 2H), 1.68-1.74 (m, 1H), 1.95-2.18 (m, 4H), 2.47-2.53 (m, 1H), 2.70-2.92 (m, 2H), 3.03 (br s, 2H), 5.32 (br s, 1H), 5.72 (d, 1H, J=9.6 Hz), 5.94 (d, 1H, J=9.6 Hz).

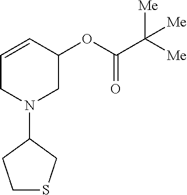 [SMILES: CC(C)(C)C(=O)OC1C=CCN(C2CCSC2)C1]

Following general procedure C and starting from 1,2,3,6-tetrahydropyridin-3-yl pivalate (188 mg, 1.03 mmol, 1.0 equiv.) and dihydrothiophen-3(2H)-one (283 μL, 3.08 mmol, 3.0 equiv.), 1-(tetrahydrothiophen-3-yl)-1,2,3,6-tetrahydropyridin-3-yl pivalate was obtained as a colorless oil (300 mg, 1.03 mmol, 100%). ¹H NMR (400 MHz, CDCl₃) δ ppm 1.20 (s, 9H), 1.82-1.95 (m, 1H), 2.25-2.34 (m, 1H), 2.48-2.54 (m, 1H), 2.74 (q, 1H, J=10.6 Hz), 2.84-2.88 (m, 2H), 3.02-3.10 (m, 3H), 3.13 (dd, 1H, J=11.9 Hz, J=1.4 Hz), 3.19 (br s, 1H), 5.29 (br s, 1H), 5.75 (d, 1H, J=9.9 Hz), 5.96 (dt, 1H, J=9.9 Hz, J=3.2 Hz).

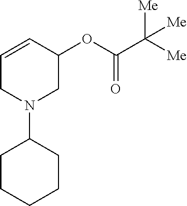 [SMILES: CC(C)(C)C(=O)OC1C=CCN(C2CCCCC2)C1]

Following general procedure C and starting from 1,2,3,6-tetrahydropyridin-3-yl pivalate (188 mg, 1.03 mmol, 1.0 equiv.) and cyclohexanone (319 μL, 3.08 mmol, 3.0 equiv.), 1-cyclohexyl-1,2,3,6-tetrahydropyridin-3-yl pivalate was obtained as a colorless oil (192 mg, 0.72 mmol, 70%). ¹H NMR (400 MHz, CDCl₃) δ ppm 1.08-1.14 (m, 1H), 1.20 (s, 9H), 1.22-1.27 (m, 4H), 1.60-1.64 (m, 1H), 1.79-1.83 (m, 3H), 2.09 (d, 1H, J=9.8 Hz), 2.39 (br s, 1H), 2.56 (dd, 1H, J=10.5 Hz, J=5.3 Hz), 2.91 (dd, 1H, J=12.0 Hz, J=4.5 Hz), 3.09 (d, 1H, J=16.5 Hz), 3.17 (d, 1H, J=16.6 Hz), 5.29 (br s, 1H), 5.70 (d, 1H, J=9.5 Hz), 5.97 (d, 1H, J=9.7 Hz); ¹³C NMR (100 MHz, CDCl₃) δ ppm 26.1, 26.2, 26.6, 27.5, 28.5, 29.4, 38.2, 48.9, 50.3, 62.9, 68.4, 125.0, 131.4, 178.7.

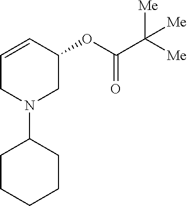 [SMILES: CC(C)(C)C(=O)O[C@H]1C=CCN(C2CCCCC2)C1]

Following general procedure C and starting from (S)-1,2,3,6-tetrahydropyridin-3-yl pivalate (197 mg, 1.07 mmol, 1.0 equiv.) and cyclohexanone (334 μL, 3.23 mmol, 3.0 equiv.), (S)-1-cyclohexyl-1,2,3,6-tetrahydropyridin-3-yl pivalate was obtained as a colorless oil (274 mg, 1.03 mmol, 96%). ¹H NMR (400 MHz, CDCl₃) δ ppm 1.08-1.14 (m, 1H), 1.20 (s, 9H), 1.22-1.27 (m, 4H), 1.60-1.64 (m, 1H), 1.79-1.83 (m, 3H), 2.09 (d, 1H, J=9.8 Hz), 2.39 (br s, 1H), 2.56 (dd, 1H, J=10.5 Hz, J=5.3 Hz), 2.91 (dd, 1H, J=12.0 Hz, J=4.5 Hz), 3.09 (d, 1H, J=16.5 Hz), 3.17 (d, 1H, J=16.6 Hz), 5.29 (br s, 1H), 5.70 (d, 1H, J=9.5 Hz), 5.97 (d, 1H, J=9.7 Hz); ¹³C NMR (100 MHz, CDCl₃) δ ppm 26.1, 26.2, 26.6, 27.5, 28.5, 29.4, 38.2, 48.9, 50.3, 62.9, 68.4, 125.0, 131.4, 178.7.

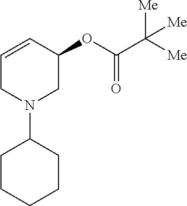 [SMILES: CC(C)(C)C(=O)O[C@@H]1C=CCN(C2CCCCC2)C1]

Following general procedure C and starting from (R)-1,2,3,6-tetrahydropyridin-3-yl pivalate (170 mg, 0.93 mmol, 1.0 equiv.) and cyclohexanone (288 μL, 2.78 mmol, 3.0 equiv.), (R)-1-cyclohexyl-1,2,3,6-tetrahydropyridin-3-yl pivalate was obtained as a colorless oil (247 mg, 0.93 mmol, 100%). ¹H NMR (400 MHz, CDCl₃) δ ppm 1.08-1.14 (m, 1H), 1.20 (s, 9H), 1.22-1.27 (m, 4H), 1.60-1.64 (m, 1H), 1.79-1.83 (m, 3H), 2.09 (d, 1H, J=9.8 Hz), 2.39 (br s, 1H), 2.56 (dd, 1H, J=10.5 Hz, J=5.3 Hz), 2.91 (dd, 1H, J=12.0 Hz, J=4.5 Hz), 3.09 (d, 1H, J=16.5 Hz), 3.17 (d, 1H, J=16.6 Hz), 5.29 (br s, 1H), 5.70 (d, 1H, J=9.5 Hz), 5.97 (d, 1H, J=9.7 Hz); ¹³C NMR (100 MHz, CDCl₃) δ ppm 26.1, 26.2, 26.6, 27.5, 28.5, 29.4, 38.2, 48.9, 50.3, 62.9, 68.4, 125.0, 131.4, 178.7.

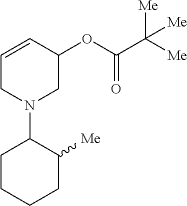 [SMILES: CC1CCCCC1N1CC=CC(OC(=O)C(C)(C)C)C1]

Following general procedure C and starting from 1,2,3,6-tetrahydropyridin-3-yl pivalate (122 mg, 0.67 mmol, 1.0 equiv.) and 3-methylcyclohexan-1-one (245 μL, 2.00 mmol, 3.0 equiv.), 1-(3-methylcyclohexyl)-1,2,3,6-tetrahydropyridin-3-yl pivalate was obtained as a colorless oil (65 mg, 0.23 mmol, 35%). ¹H NMR (400 MHz, CDCl₃) δ ppm 0.90 (dd, 1H, J=8.6 Hz, J=7.1 Hz), 1.02 (d, 1H, J=6.3 Hz), 1.20 (s, 9H), 1.49-1.70 (m, 8H), 2.50 (br s, 1H), 2.64 (d, 1H, J=11.5 Hz), 2.76 (br s, 1H), 2.99 (br s, 1H), 3.15 (d, 1H, J=15.9 Hz), 5.26 (br s, 1H), 5.73 (d, 1H, J=9.8 Hz), 5.97 (d, 1H, J=9.8 Hz).

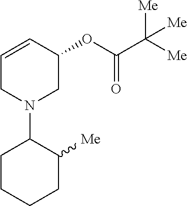 [SMILES: CC1CCCCC1N1CC=C[C@H](OC(=O)C(C)(C)C)C1]

Following general procedure C and starting from (S)-1,2,3,6-tetrahydropyridin-3-yl pivalate (200 mg, 1.09 mmol, 1.0 equiv.) and 3-methylcyclohexan-1-one (397 μL, 3.27 mmol, 3.0 equiv.), (S)-1-(3-methylcyclohexyl)-1,2,3,6-tetrahydropyridin-3-yl pivalate was obtained as a yellow oil (123 mg, 0.44 mmol, 40%). ¹H NMR (400 MHz, CDCl₃) δ ppm 0.90 (dd, 1H, J=8.6 Hz, J=7.1 Hz), 1.02 (d, 1H, J=6.3 Hz), 1.20 (s, 9H), 1.49-1.70 (m, 8H), 2.50 (br s, 1H), 2.64 (d, 1H, J=11.5 Hz), 2.76 (br s, 1H), 2.99 (br s, 1H), 3.15 (d, 1H, J=15.9 Hz), 5.26 (br s, 1H), 5.73 (d, 1H, J=9.8 Hz), 5.97 (d, 1H, J=9.8 Hz).

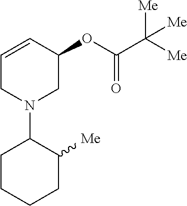 [SMILES: CC1CCCCC1N1CC=C[C@@H](OC(=O)C(C)(C)C)C1]

Following general procedure C and starting from (R)-1,2,3,6-tetrahydropyridin-3-yl pivalate (199 mg, 1.09 mmol, 1.0 equiv.) and 3-methylcyclohexan-1-one (395 μL, 3.26 mmol, 3.0 equiv.), (R)-1-(3-methylcyclohexyl)-1,2,3,6-tetrahydropyridin-3-yl pivalate was obtained as a yellow oil (102 mg, 0.36 mmol, 34%). ¹H NMR (400 MHz, CDCl₃) δ ppm 0.90 (dd, 1H, J=8.6 Hz, J=7.1 Hz), 1.02 (d, 1H, J=6.3 Hz), 1.20 (s, 9H), 1.49-1.70 (m, 8H), 2.50 (br s, 1H), 2.64 (d, 1H, J=11.5 Hz), 2.76 (br s, 1H), 2.99 (br s, 1H), 3.15 (d, 1H, J=15.9 Hz), 5.26 (br s, 1H), 5.73 (d, 1H, J=9.8 Hz), 5.97 (d, 1H, J=9.8 Hz).

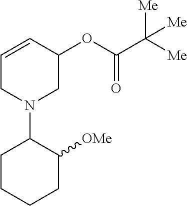 [SMILES: COC1CCCCC1N1CC=CC(OC(=O)C(C)(C)C)C1]

Following general procedure C and starting from 1,2,3,6-tetrahydropyridin-3-yl pivalate (182 mg, 0.99 mmol, 1.0 equiv.) and 2-methoxycyclohexan-1-one (374 μL, 2.98 mmol, 3.0 equiv.), 1-(2-methoxycyclohexyl)-1,2,3,6-tetrahydropyridin-3-yl pivalate was obtained as a colorless oil (103 mg, 0.35 mmol, 35%). ¹H NMR (400 MHz, CDCl₃) δ ppm 1.20 (s, 9H), 1.49-1.92 (m, 7H), 2.17 (br s, 1H), 2.47 (br s, 1H), 2.70 (br s, 1H), 3.03 (br s, 1H), 3.20 (d, 1H, J=9.1 Hz), 3.35 (d, 1H, J=10.9 Hz), 3.38 (s, 3H), 5.26 (br s, 1H), 5.69 (br s, 1H), 5.91-5.98 (m, 1H).

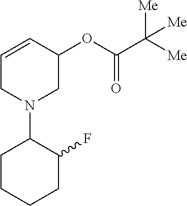 [SMILES: CC(C)(C)C(=O)OC1C=CCN(C2CCCCC2F)C1]

Following general procedure C and starting from 1,2,3,6-tetrahydropyridin-3-yl pivalate (182 mg, 0.99 mmol, 1.0 equiv.) and 2-fluorocyclohexan-1-one (333 μL, 2.98 mmol, 3.0 equiv.), 1-(2-fluorocyclohexyl)-1,2,3,6-tetrahydropyridin-3-yl pivalate was obtained as a colorless oil (202 mg, 0.71 mmol, 72%). ¹H NMR (400 MHz, CDCl₃) δ ppm 1.20 (s, 9H), 1.33-1.40 (m, 2H), 1.52-1.56 (m, 2H), 1.61-1.66 (m, 1H), 1.71-1.76 (m, 1H), 1.80-1.89 (m, 3H), 2.17-2.23 (m, 1H), 2.82 (br s, 1H), 3.14 (br s, 1H), 3.34 (br s, 2H), 4.69 (ddd, 1H, J=47.8 Hz, J=9.3 Hz, J=4.6 Hz), 5.36 (br s, 1H), 5.79 (br s, 1H), 5.96 (d, 1H, J=9.7 Hz); ¹⁹F NMR (376 MHz, CDCl₃) δ ppm −68.2.

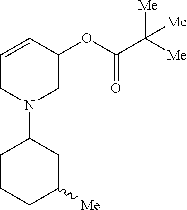 [SMILES: CC1CCCC(N2CC=CC(OC(=O)C(C)(C)C)C2)C1]

Following general procedure C and starting from 1,2,3,6-tetrahydropyridin-3-yl pivalate (122 mg, 0.67 mmol, 1.0 equiv.) and 3-methylcyclohexan-1-one (245 μL, 2.00 mmol, 3.0 equiv.), 1-(3-methylcyclohexyl)-1,2,3,6-tetrahydropyridin-3-yl pivalate was obtained as a colorless oil (65 mg, 0.23 mmol, 35%). ¹H NMR (400 MHz, CDCl₃) δ ppm 0.90 (dd, 3H, J=8.6 Hz, J=7.1 Hz), 1.02 (d, 1H, J=6.3 Hz), 1.20 (s, 9H), 1.49-1.70 (m, 8H), 2.50 (br s, 1H), 2.64 (d, 1H, J=12.2 Hz), 2.77 (br s, 1H), 2.99 (br s, 1H), 3.16 (d, 1H, J=15.9 Hz), 5.26 (br s, 1H), 5.73 (d, 1H, J=9.8 Hz), 5.97 (d, 1H, J=9.8 Hz).

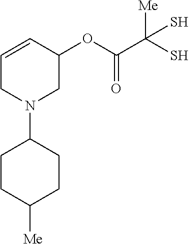 [SMILES: CC1CCC(N2CC=CC(OC(=O)C(C)(S)S)C2)CC1]

Following general procedure C and starting from 1,2,3,6-tetrahydropyridin-3-yl pivalate (122 mg, 0.67 mmol, 1.0 equiv.) and 4-methylcyclohexan-1-one (245 μL, 2.00 mmol, 3.0 equiv.), 1-(4-methylcyclohexyl)-1,2,3,6-tetrahydropyridin-3-yl pivalate was obtained as a colorless oil (58 mg, 0.21 mmol, 31%). ¹H NMR (400 MHz, CDCl₃) δ ppm 0.91 (d, 3H, J=6.9 Hz), 1.20 (s, 9H), 1.43-1.51 (m, 6H), 1.65-1.70 (m, 3H), 2.29-2.36 (m, 1H), 2.65 (br s, 1H), 2.80 (br s, 1H), 3.00 (d, 1H, J=14.8 Hz), 3.17 (d, 1H, J=16.1 Hz), 5.27 (br s, 1H), 5.74 (d, 1H, J=9.9 Hz), 5.97 (dt, 1H, J=9.9 Hz, J=3.2 Hz).

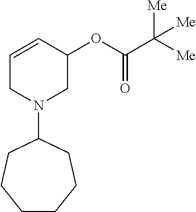 [SMILES: CC(C)(C)C(=O)OC1C=CCN(C2CCCCCC2)C1]

Following general procedure C and starting from 1,2,3,6-tetrahydropyridin-3-yl pivalate (156 mg, 0.85 mmol, 1.0 equiv.) and cycloheptanone (301 μL, 2.55 mmol, 3.0 equiv.), 1-cycloheptyl-1,2,3,6-tetrahydropyridin-3-yl pivalate was obtained as a colorless oil (90 mg, 0.32 mmol, 38%). ¹H NMR (400 MHz, CDCl₃) δ ppm 1.20 (s, 9H), 1.46-1.54 (m, 6H), 1.67-1.70 (m, 2H), 1.84-1.86 (m, 2H), 2.57 (br s, 1H), 2.67 (br s, 1H), 2.90 (br s, 1H), 3.13 (d, 2H, J=10.4 Hz), 5.30 (br s, 1H), 5.70 (d, 1H, J=9.9 Hz), 5.96 (d, 1H, J=9.9 Hz).

Step 6. Formation of the final product

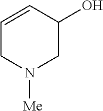

Following general procedure E and starting from 1-methyl-1,2,3,6-tetrahydropyridin-3-yl pivalate (536 mg, 2.72 mmol, 1.0 equiv.), 1-methyl-1,2,3,6-tetrahydropyridin-3-ol was obtained as a yellow oil (207 mg, 1.83 mmol, 67%). ¹H NMR (400 MHz, CDCl₃) δ ppm 2.35 (s, 3H), 2.46 (dd, 1H, J=11.7 Hz, J=3.4 Hz), 2.65-2.70 (m, 2H), 3.07 (d, 1H, J=16.8 Hz), 4.07 (s, 1H), 5.82 (d, 1H, J=11.8 Hz), 5.88 (d, 1H, J=11.7 Hz); ¹³C NMR (100 MHz, CDCl₃) δ ppm 46.1, 54.8, 60.4, 64.9, 128.0, 129.0.

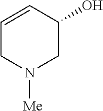

Following general procedure E and starting from (S)-1-methyl-1,2,3,6-tetrahydropyridin-3-yl pivalate (315 mg, 1.60 mmol, 1.0 equiv.), (S)-1-methyl-1,2,3,6-tetrahydropyridin-3-ol was obtained as a yellow oil (92 mg, 0.81 mmol, 51%). ¹H NMR (400 MHz, CDCl₃) δ ppm 2.35 (s, 3H), 2.46 (dd, 1H, J=11.7 Hz, J=3.4 Hz), 2.65-2.70 (m, 2H), 3.07 (d, 1H, J=16.8 Hz), 4.07 (s, 1H), 5.82 (d, 1H, J=11.8 Hz), 5.88 (d, 1H, J=11.7 Hz); ¹³C NMR (100 MHz, CDCl₃) δ ppm 46.1, 54.8, 60.4, 64.9, 128.0, 129.0.

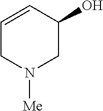

Following general procedure E and starting from (R)-1-methyl-1,2,3,6-tetrahydropyridin-3-yl pivalate (271 mg, 1.37 mmol, 1.0 equiv.), (R)-1-methyl-1,2,3,6-tetrahydropyridin-3-ol was obtained as a yellow oil (121 mg, 1.07 mmol, 78%). ¹H NMR (400 MHz, CDCl₃) δ ppm 2.35 (s, 3H), 2.46 (dd, 1H, J=11.7 Hz, J=3.4 Hz), 2.65-2.70 (m, 2H), 3.07 (d, 1H, J=16.8 Hz), 4.07 (s, 1H), 5.82 (d, 1H, J=11.8 Hz), 5.88 (d, 1H, J=11.7 Hz); ¹³C NMR (100 MHz, CDCl₃) δ ppm 46.1, 54.8, 60.4, 64.9, 128.0, 129.0.

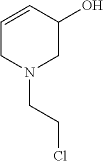

Following general procedure E and starting from 1-(2-chloroethyl)-1,2,3,6-tetrahydropyridin-3-yl pivalate (54 mg, 0.22 mmol, 1.0 equiv.), 1-(2-chloroethyl)-1,2,3,6-tetrahydropyridin-3-ol was obtained as a white solid (5 mg, 0.03 mmol, 14%). ¹H NMR (400 MHz, CDCl₃) δ ppm 2.18 (d, 1H, J=9.4 Hz), 2.59 (dd, 1H, J=11.4 Hz, J=2.7 Hz), 2.80-2.85 (m, 2H), 2.89 (d, 1H, J=16.7 Hz), 3.22 (dd, 1H, J=16.7 Hz, J=4.0 Hz), 3.61 (t, 2H, J=6.9 Hz), 4.06 (br s, 1H), 5.83 (dq, 1H, J=9.9 Hz, J=1.9 Hz), 5.92 (dt, 1H, J=9.9 Hz, J=2.1 Hz); ¹³C NMR (100 MHz, CDCl₃) δ ppm 41.5, 53.1, 58.0, 59.3, 64.7, 128.2, 128.9.

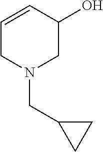 [SMILES: OC1C=CCN(CC2CC2)C1]

Following general procedure E and starting from 1-(cyclopropylmethyl)-1,2,3,6-tetrahydropyridin-3-yl pivalate (281 mg, 1.18 mmol, 1.0 equiv.), 1-(cyclopropylmethyl)-1,2,3,6-tetrahydropyridin-3-ol was obtained as a yellow oil (118 mg, 0.77 mmol, 65%). ¹H NMR (400 MHz, CDCl₃) δ ppm 0.12 (q, 2H, J=4.7 Hz), 0.53 (q, 2H, J=8.0 Hz), 0.90 (sept.d, 1H, J=6.7 Hz, J=1.4 Hz), 2.35 (d, 2H, J=6.5 Hz), 2.56 (dd, 1H, J=8.2 Hz, J=3.2 Hz), 2.82 (d, 1H, J=17.0 Hz), 2.88 (dd, 1H, J=11.5 Hz, J=3.4 Hz), 3.25 (dd, 1H, J=17.2 Hz, J=3.4 Hz), 4.09 (br s, 1H), 5.85 (d, 1H, J=9.8 Hz), 5.90 (d, 1H, J=10.7 Hz); ¹³C NMR (100 MHz, CDCl₃) δ ppm 4.2, 4.3, 8.7, 53.0, 58.2, 63.4, 64.8, 128.2, 129.2.

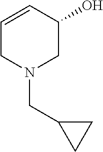 [SMILES: O[C@H]1C=CCN(CC2CC2)C1]

Following general procedure E and starting from (S)-1-(cyclopropylmethyl)-1,2,3,6-tetrahydropyridin-3-yl pivalate (151 mg, 0.64 mmol, 1.0 equiv.), (S)-1-(cyclopropylmethyl)-1,2,3,6-tetrahydropyridin-3-ol was obtained as a yellow oil (77 mg, 0.50 mmol, 79%). ¹H NMR (400 MHz, CDCl₃) δ ppm 0.12 (q, 2H, J=4.7 Hz), 0.53 (q, 2H, J=8.0 Hz), 0.90 (sept.d, 1H, J=6.7 Hz, J=1.4 Hz), 2.35 (d, 2H, J=6.5 Hz), 2.56 (dd, 1H, J=8.2 Hz, J=3.2 Hz), 2.82 (d, 1H, J=17.0 Hz), 2.88 (dd, 1H, J=11.5 Hz, J=3.4 Hz), 3.25 (dd, 1H, J=17.2 Hz, J=3.4 Hz), 4.09 (br s, 1H), 5.85 (d, 1H, J=9.8 Hz), 5.90 (d, 1H, J=10.7 Hz); ¹³C NMR (100 MHz, CDCl₃) δ ppm 4.2, 4.3, 8.7, 53.0, 58.2, 63.4, 64.8, 128.2, 129.2.

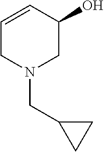 [SMILES: O[C@@H]1C=CCN(CC2CC2)C1]

Following general procedure E and starting from (R)-1-(cyclopropylmethyl)-1,2,3,6-tetrahydropyridin-3-yl pivalate (249 mg, 1.05 mmol, 1.0 equiv.), (R)-1-(cyclopropylmethyl)-1,2,3,6-tetrahydropyridin-3-ol was obtained as a yellow oil (95 mg, 0.62 mmol, 59%). ¹H NMR (400 MHz, CDCl₃) δ ppm 0.12 (q, 2H, J=4.7 Hz), 0.53 (q, 2H, J=8.0 Hz), 0.90 (sept.d, 1H, J=6.7 Hz, J=1.4 Hz), 2.35 (d, 2H, J=6.5 Hz), 2.56 (dd, 1H, J=8.2 Hz, J=3.2 Hz), 2.82 (d, 1H, J=17.0 Hz), 2.88 (dd, 1H, J=11.5 Hz, J=3.4 Hz), 3.25 (dd, 1H, J=17.2 Hz, J=3.4 Hz), 4.09 (br s, 1H), 5.85 (d, 1H, J=9.8 Hz), 5.90 (d, 1H, J=10.7 Hz); ¹³C NMR (100 MHz, CDCl₃) δ ppm 4.2, 4.3, 8.7, 53.0, 58.2, 63.4, 64.8, 128.2, 129.2.

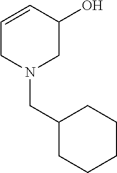 [SMILES: OC1C=CCN(CC2CCCCC2)C1]

Following general procedure E and starting from 1-(cyclohexylmethyl)-1,2,3,6-tetrahydropyridin-3-yl pivalate (109 mg, 0.39 mmol, 1.0 equiv.), 1-(cyclohexylmethyl)-1,2,3,6-tetrahydropyridin-3-ol was obtained as a yellow oil (63 mg, 0.32 mmol, 82%). ¹H NMR (400 MHz, CDCl₃) δ ppm 0.87 (qd, 2H, J=15.6 Hz, J=2.7 Hz), 1.16-1.26 (m, 3H), 1.49-1.56 (m, 1H), 1.64-1.73 (m, 3H), 1.76-1.80 (m, 2H), 2.22 (t, 2H, J=7.8 Hz), 2.40 (dd, 1H, J=11.4 Hz, J=2.7 Hz), 2.67 (d, 1H, J=16.9 Hz), 2.78 (dd, 1H, J=11.5 Hz, J=3.1 Hz), 3.13 (dd, 1H, J=17.2 Hz, J=4.2 Hz), 4.01 (q, 1H, J=2.5 Hz), 5.82 (dq, 1H, J=9.8 Hz, J=2.2 Hz), 5.90 (dt, 1H, J=9.8 Hz, J=2.2 Hz); ¹³C NMR (100 MHz, CDCl₃) δ ppm 26.4, 28.1, 32.1, 32.2, 35.4, 54.0, 58.6, 64.9, 65.4, 128.2, 129.5.

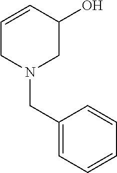 [SMILES: OC1C=CCN(Cc2ccccc2)C1]

Following general procedure E and starting from 1-benzyl-1,2,3,6-tetrahydropyridin-3-yl pivalate (197 mg, 0.72 mmol, 1.0 equiv.), 1-benzyl-1,2,3,6-tetrahydropyridin-3-ol was obtained as a yellow oil (84 mg, 0.44 mmol, 61%). ¹H NMR (400 MHz, CDCl₃) δ ppm 2.51 (dd, 1H, J=11.4 Hz, J=3.1 Hz), 2.74-2.82 (m, 2H), 3.13 (dd, 1H, J=17.0 Hz, J=4.0 Hz), 3.61 (s, 2H), 4.05 (br s, 1H), 5.82 (dq, 1H, J=9.8 Hz, J=4.0 Hz), 5.90 (dt, 1H, J=9.8 Hz, J=2.2 Hz), 7.25-7.28 (m, 1H), 7.31-7.33 (m, 4).

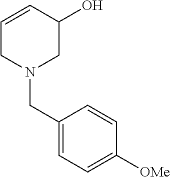 [SMILES: COc1ccc(CN2CC=CC(O)C2)cc1]

Following general procedure E and starting from 1-(4-methoxybenzyl)-1,2,3,6-tetrahydropyridin-3-yl pivalate (207 mg, 0.68 mmol, 1.0 equiv.), 1-(4-methoxybenzyl)-1,2,3,6-tetrahydropyridin-3-ol was obtained as an orange oil (50 mg, 0.23 mmol, 34%). ¹H NMR (400 MHz, CDCl₃) δ ppm 2.50 (dt, 1H, J=11.5 Hz, J=3.3 Hz), 2.74-2.82 (m, 2H), 3.13 (d, 1H, J=16.8 Hz), 3.57 (d, 2H, J=5.9 Hz), 3.80 (s, 3H), 4.05 (br s, 1H), 5.81 (d, 1H, J=9.9 Hz), 5.90 (d, 1H, J=9.7 Hz), 6.86 (d, 2H, J=8.5 Hz), 7.24 (d, 2H, J=8.5 Hz); ¹³C NMR (100 MHz, CDCl₃) δ ppm 52.9, 55.6, 57.7, 62.1, 64.8, 114.1, 114.7, 128.3, 129.1, 129.2, 130.6, 130.7.

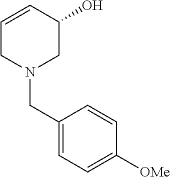 [SMILES: COc1ccc(CN2CC=C[C@H](O)C2)cc1]

Following general procedure E and starting from (S)-1-(4-methoxybenzyl)-1,2,3,6-tetrahydropyridin-3-yl pivalate (251 mg, 0.83 mmol, 1.0 equiv.), (S)-1-(4-methoxybenzyl)-1,2,3,6-tetrahydropyridin-3-ol was obtained as a pinkish oil (28 mg, 0.13 mmol, 16%). ¹H NMR (400 MHz, CDCl₃) δ ppm 2.50 (dt, 1H, J=11.5 Hz, J=3.3 Hz), 2.74-2.82 (m, 2H), 3.13 (d, 1H, J=16.8 Hz), 3.57 (d, 2H, J=5.9 Hz), 3.80 (s, 3H), 4.05 (br s, 1H), 5.81 (d, 1H, J=9.9 Hz), 5.90 (d, 1H, J=9.7 Hz), 6.86 (d, 2H, J=8.5 Hz), 7.24 (d, 2H, J=8.5 Hz); ¹³C NMR (100 MHz, CDCl₃) δ ppm 52.9, 55.6, 57.7, 62.1, 64.8, 114.1, 114.7, 128.3, 129.1, 129.2, 130.6, 130.7.

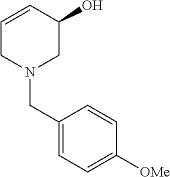 [SMILES: COc1ccc(CN2CC=C[C@@H](O)C2)cc1]

Following general procedure E and starting from (R)-1-(4-methoxybenzyl)-1,2,3,6-tetrahydropyridin-3-yl pivalate (265 mg, 0.87 mmol, 1.0 equiv.), (R)-1-(4-methoxybenzyl)-1,2,3,6-tetrahydropyridin-3-ol was obtained as an orange oil (70 mg, 0.32 mmol, 36%). ¹H NMR (400 MHz, CDCl₃) δ ppm 2.50 (dt, 1H, J=11.5 Hz, J=3.3 Hz), 2.74-2.82 (m, 2H), 3.13 (d, 1H, J=16.8 Hz), 3.57 (d, 2H, J=5.9 Hz), 3.80 (s, 3H), 4.05 (br s, 1H), 5.81 (d, 1H, J=9.9 Hz), 5.90 (d, 1H, J=9.7 Hz), 6.86 (d, 2H, J=8.5 Hz), 7.24 (d, 2H, J=8.5 Hz); ¹³C NMR (100 MHz, CDCl₃) δ ppm 52.9, 55.6, 57.7, 62.1, 64.8, 114.1, 114.7, 128.3, 129.1, 129.2, 130.6, 130.7.

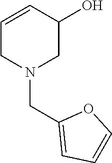 [SMILES: OC1C=CCN(Cc2ccco2)C1]

Following general procedure E and starting from 1-(furan-2-ylmethyl)-1,2,3,6-tetrahydropyridin-3-yl pivalate (314 mg, 1.19 mmol, 1.0 equiv.), 1-(furan-2-ylmethyl)-1,2,3,6-tetrahydropyridin-3-ol was obtained as a red oil (117 mg, 0.65 mmol, 55%). ¹H NMR (400 MHz, CDCl₃) δ ppm 25.2 (br s, 1H), 2.57 (dd, 1H, J=11.5 Hz, J=3.3 Hz), 2.78 (dd, 1H, J=11.5 Hz, J=3.5 Hz), 2.85 (d, 1H, J=16.8 Hz), 3.13 (dd, 1H, J=16.9 Hz, J=3.7 Hz), 3.66 (s, 2H), 4.08 (br s, 1H), 5.82 (dq, 1, J=9.8 Hz, J=2.4 Hz), 5.87 (dt, 1H, J=9.9 Hz, J=1.9 Hz), 6.22 (d, 1H, J=3.2 Hz), 6.32 (dd, 1H, J=3.1 Hz, J=1.8 Hz), 7.37 (d, 1H, J=1.8 Hz); ¹³C NMR (100 MHz, CDCl₃) δ ppm 52.4, 54.4, 57.6, 64.9, 109.2, 110.5, 128.2, 128.9, 142.6, 151.8.

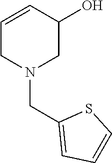 [SMILES: OC1C=CCN(Cc2cccs2)C1]

Following general procedure E and starting from 1-(thiophen-2-ylmethyl)-1,2,3,6-tetrahydropyridin-3-yl pivalate (351 mg, 1.26 mmol, 1.0 equiv.), 1-(thiophen-2-ylmethyl)-1,2,3,6-tetrahydropyridin-3-ol was obtained as an orange oil (118 mg, 0.61 mmol, 48%). ¹H NMR (400 MHz, CDCl₃) δ ppm 2.33 (br s, 1H), 2.52 (dd, 1H, J=11.4 Hz, J=3.1 Hz), 2.81-2.86 (m, 2H), 3.21 (dd, 1H, J=16.9 Hz, J=4.0 Hz), 3.80 (d, 1H, J=13.8 Hz), 3.86 (d, 1H, J=14.0 Hz), 4.06 (q, 1H, J=2.5 Hz), 5.83 (dq, 1H, J=9.9 Hz, J=2.3 Hz), 5.91 (dt, 1H, J=9.8 Hz, J=2.2 Hz), 6.91-6.96 (m, 2H), 7.24 (dd, 1H, J=4.9 Hz, J=1.1 Hz); ¹³C NMR (100 MHz, CDCl₃) δ ppm 52.5, 56.4, 56.9, 64.5, 125.2, 126.0, 126.4, 127.9, 128.6, 141.3.

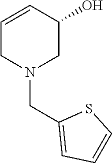 [SMILES: O[C@H]1C=CCN(Cc2cccs2)C1]

Following general procedure E and starting from (S)-1-(thiophen-2-ylmethyl)-1,2,3,6-tetrahydropyridin-3-yl pivalate (182 mg, 0.65 mmol, 1.0 equiv.), (S)-1-(thiophen-2-ylmethyl)-1,2,3,6-tetrahydropyridin-3-ol was obtained as an orange oil (105 mg, 0.54 mmol, 82%). ¹H NMR (400 MHz, CDCl₃) δ ppm 2.33 (br s, 1H), 2.52 (dd, 1H, J=11.4 Hz, J=3.1 Hz), 2.81-2.86 (m, 2H), 3.21 (dd, 1H, J=16.9 Hz, J=4.0 Hz), 3.80 (d, 1H, J=13.8 Hz), 3.86 (d, 1H, J=14.0 Hz), 4.06 (q, 1H, J=2.5 Hz), 5.83 (dq, 1H, J=9.9 Hz, J=2.3 Hz), 5.91 (dt, 1H, J=9.8 Hz, J=2.2 Hz), 6.91-6.96 (m, 2H), 7.24 (dd, 1H, J=4.9 Hz, J=1.1 Hz); ¹³C NMR (100 MHz, CDCl₃) δ ppm 52.5, 56.4, 56.9, 64.5, 125.2, 126.0, 126.4, 127.9, 128.6, 141.3.

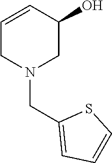 [SMILES: O[C@@H]1C=CCN(Cc2cccs2)C1]

Following general procedure E and starting from (R)-1-(thiophen-2-ylmethyl)-1,2,3,6-tetrahydropyridin-3-yl pivalate (293 mg, 1.05 mmol, 1.0 equiv.), (R)-1-(thiophen-2-ylmethyl)-1,2,3,6-tetrahydropyridin-3-ol was obtained as an orange oil (124 mg, 0.64 mmol, 61%). ¹H NMR (400 MHz, CDCl₃) δ ppm 2.33 (br s, 1H), 2.52 (dd, 1H, J=11.4 Hz, J=3.1 Hz), 2.81-2.86 (m, 2H), 3.21 (dd, 1H, J=16.9 Hz, J=4.0 Hz), 3.80 (d, 1H, J=13.8 Hz), 3.86 (d, 1H, J=14.0 Hz), 4.06 (q, 1H, J=2.5 Hz), 5.83 (dq, 1H, J=9.9 Hz, J=2.3 Hz), 5.91 (dt, 1H, J=9.8 Hz, J=2.2 Hz), 6.91-6.96 (m, 2H), 7.24 (dd, 1H, J=4.9 Hz, J=1.1 Hz); ¹³C NMR (100 MHz, CDCl₃) δ ppm 52.5, 56.4, 56.9, 64.5, 125.2, 126.0, 126.4, 127.9, 128.6, 141.3.

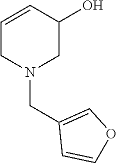 [SMILES: OC1C=CCN(Cc2ccoc2)C1]

Following general procedure E and starting from 1-(furan-3-ylmethyl)-1,2,3,6-tetrahydropyridin-3-yl pivalate (226 mg, 0.86 mmol, 1.0 equiv.), 1-(furan-3-ylmethyl)-1,2,3,6-tetrahydropyridin-3-ol was obtained as a colorless oil (117 mg, 0.65 mmol, 76%). ¹H NMR (400 MHz, CDCl₃) δ ppm 2.48 (dd, 1H, J=11.4 Hz, J=3.2 Hz), 2.74-2.81 (m, 2H), 3.14 (dd, 1H, J=16.8 Hz, J=3.8 Hz), 3.46 (d, 1H, J=13.4 Hz), 3.51 (d, 1H, J=13.5 Hz), 4.06 (q, 1H, J=3.5 Hz), 5.83 (dq, 1H, J=9.8 Hz, J=2.2 Hz), 5.89 (dt, 1H, J=9.8 Hz, J=2.1 Hz), 6.39 (s, 1H), 7.34-7.39 (m, 2H); ¹³C NMR (100 MHz, CDCl₃) δ ppm 52.6, 52.9, 57.6, 64.9, 111.5, 121.6, 128.2, 129.1, 141.1, 143.5.

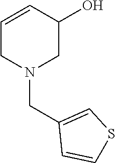 [SMILES: OC1C=CCN(Cc2ccsc2)C1]

Following general procedure E and starting from 1-(thiophen-3-ylmethyl)-1,2,3,6-tetrahydropyridin-3-yl pivalate (327 mg, 1.17 mmol, 1.0 equiv.), 1-(thiophen-3-ylmethyl)-1,2,3,6-tetrahydropyridin-3-ol was obtained as an orange oil (145 mg, 0.74 mmol, 63%). ¹H NMR (400 MHz, CDCl₃) δ ppm 2.35 (br s, 1H), 2.49 (dd, 1H, J=11.4 Hz, J=3.1 Hz), 2.75-2.81 (m, 2H), 3.13 (dd, 1H, J=16.9 Hz, J=3.9 Hz), 3.62 (d, 1H, J=13.3 Hz), 3.66 (d, 1H, J=13.4 Hz), 4.06 (q, 1H, J=3.2 Hz), 5.82 (dq, 1H, J=9.8 Hz, J=2.2 Hz), 5.89 (dt, 1H, J=9.9 Hz, J=2.1 Hz), 7.06 (d, 1H, J=4.9 Hz), 7.13 (d, 1H, J=2.4 Hz), 7.28 (dd, 1H, J=4.9 Hz, J=3.0 Hz); ¹³C NMR (100 MHz, CDCl₃) δ ppm 53.0, 57.3, 57.8, 64.9, 123.2, 126.0, 128.2, 128.7, 129.1, 139.1.

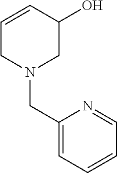 [SMILES: OC1C=CCN(Cc2ccccn2)C1]

Following general procedure E and starting from 1-(pyridin-2-ylmethyl)-1,2,3,6-tetrahydropyridin-3-yl pivalate (142 mg, 0.52 mmol, 1.0 equiv.), 1-(pyridin-2-ylmethyl)-1,2,3,6-tetrahydropyridin-3-ol was obtained as a yellow oil (51 mg, 0.27 mmol, 52%). ¹H NMR (400 MHz, CDCl₃) δ ppm 2.27 (br s, 1H), 2.59 (dd, 1H, J=11.5 Hz, J=3.1 Hz), 2.81 (dd, 1H, J=11.5 Hz, J=3.4 Hz), 2.89 (d, 1H, J=16.9 Hz), 3.20 (dd, 1H, J=16.8 Hz, J=3.9 Hz), 3.73 (d, 1H, J=13.8 Hz), 3.81 (d, 1H, J=13.8 Hz), 4.07 (br s, 1H), 5.83 (dq, 1H, J=9.8 Hz, J=2.3 Hz), 5.92 (dt, 1H, J=9.9 Hz, J=2.2 Hz), 7.17 (dd, 1H, J=5.0 Hz, J=7.0 Hz), 7.44 (d, 1H, J=7.8 Hz), 7.66 (td, 1H, J=7.7 Hz, J=1.7 Hz), 8.55 (d, 1H, J=4.8 Hz); ¹³C NMR (100 MHz, CDCl₃) δ ppm 53.3, 58.0, 64.2, 64.9, 122.6, 123.5, 128.2, 129.1, 136.9, 1496, 158.7.

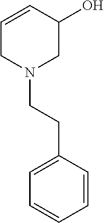 [SMILES: OC1C=CCN(CCc2ccccc2)C1]

Following general procedure E and starting from 1-phenethyl-1,2,3,6-tetrahydropyridin-3-yl pivalate (304 mg, 1.06 mmol, 1.0 equiv.), 1-phenethyl-1,2,3,6-tetrahydropyridin-3-ol was obtained as a yellow oil (152 mg, 0.75 mmol, 71%). ¹H NMR (400 MHz, CDCl₃) δ ppm 2.36 (br s, 1H), 2.59 (dd, 1H, J=11.4 Hz, J=3.1 Hz), 2.73-2.79 (m, 2H), 2.86-2.90 (m, 4H), 3.27 (dd, 1H, J=16.8 Hz, J=3.9 Hz), 4.11 (br s, 1H), 5.89 (dq, 1H, J=9.8 Hz, J=2.2 Hz), 5.95 (dt, 1H, J=9.8 Hz, J=2.1 Hz), 7.22-7.27 (m, 2H), 7.30-7.36 (m, 3H); ¹³C NMR (100 MHz, CDCl₃) δ ppm 91.0, 110.3, 115.2, 117.0, 121.9, 183.5, 185.3, 185.8, 186.1, 186.2, 197.6.

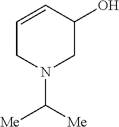

Following general procedure E and starting from 1-isopropyl-1,2,3,6-tetrahydropyridin-3-yl pivalate (218 mg, 0.97 mmol, 1.0 equiv.), 1-isopropyl-1,2,3,6-tetrahydropyridin-3-ol was obtained as a yellow oil (41 mg, 0.29 mmol, 30%). ¹H NMR (400 MHz, CDCl₃) δ ppm 1.08 (t, 6H, J=7.2 Hz), 2.56 (dd, 1H, J=11.5 Hz, J=3.1 Hz), 2.81 (dd, 1H, J=12.0 Hz, J=3.9 Hz), 2.86 (quint., 1H, J=6.6 Hz), 2.99 (d, 1H, J=16.8 Hz), 3.17 (dd, 1H, J=16.8 Hz, J=3.8 Hz), 4.08 (br s, 1H), 5.84 (d, 1H, J=9.6 Hz), 5.90 (d, 1H, J=9.4 Hz); ¹³C NMR (100 MHz, CDCl₃) δ ppm 18.0, 18.8, 48.7, 53.3, 54.3, 64.7, 128.3, 129.2.

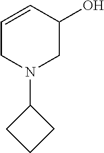 [SMILES: OC1C=CCN(C2CCC2)C1]

Following general procedure E and starting from 1-cyclobutyl-1,2,3,6-tetrahydropyridin-3-yl pivalate (185 mg, 0.78 mmol, 1.0 equiv.), 1-cyclobutyl-1,2,3,6-tetrahydropyridin-3-ol was obtained as a yellow oil (29 mg, 0.19 mmol, 24%). ¹H NMR (400 MHz, CDCl₃) δ ppm 1.63-1.76 (m, 2H), 1.95 (sept., 2H, J=10.3 Hz), 2.01-2.08 (m, 2H), 2.34 (dd, 1H, J=11.5 Hz, J=3.4 Hz), 2.61 (d, 1H, J=16.7 Hz), 2.73 (dd, 1H, J=11.5 Hz, J=3.3 Hz), 2.89 (quint., 1H, J=7.8 Hz), 3.12 (dd, 1H, J=16.9 Hz, J=3.9 Hz), 4.08 (br s, 1H), 5.83 (dq, 1H, J=9.9 Hz, J=1.8 Hz), 5.90 (dt, 1H, J=9.9 Hz, J=2.2 Hz); ¹³C NMR (100 MHz, CDCl₃) δ ppm 14.7, 27.1, 27.3, 27.8, 49.4, 54.3, 60.0, 64.3, 128.3, 128.4.

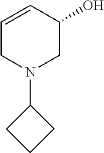 [SMILES: O[C@H]1C=CCN(C2CCC2)C1]

Following general procedure E and starting from (R)-1-cyclobutyl-1,2,3,6-tetrahydropyridin-3-yl pivalate (184 mg, 0.77 mmol, 1.0 equiv.), (R)-1-cyclobutyl-1,2,3,6-tetrahydropyridin-3-ol was obtained as a colorless oil (32 mg, 0.21 mmol, 27%). ¹H NMR (400 MHz, CDCl₃) δ ppm 1.63-1.76 (m, 2H), 1.95 (sept., 2H, J=10.3 Hz), 2.01-2.08 (m, 2H), 2.34 (dd, 1H, J=11.5 Hz, J=3.4 Hz), 2.61 (d, 1H, J=16.7 Hz), 2.73 (dd, 1H, J=11.5 Hz, J=3.3 Hz), 2.89 (quint., 1H, J=7.8 Hz), 3.12 (dd, 1H, J=16.9 Hz, J=3.9 Hz), 4.08 (br s, 1H), 5.83 (dq, 1H, J=9.9 Hz, J=1.8 Hz), 5.90 (dt, 1H, J=9.9 Hz, J=2.2 Hz); ¹³C NMR (100 MHz, CDCl₃) δ ppm 14.7, 27.1, 27.3, 27.8, 49.4, 54.3, 60.0, 64.3, 128.3, 128.4.

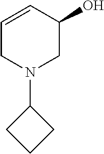 [SMILES: O[C@@H]1C=CCN(C2CCC2)C1]

Following general procedure E and starting from (S)-1-cyclobutyl-1,2,3,6-tetrahydropyridin-3-yl pivalate (84 mg, 0.36 mmol, 1.0 equiv.), (S)-1-cyclobutyl-1,2,3,6-tetrahydropyridin-3-ol was obtained as a yellow oil (27 mg, 0.18 mmol, 50%). ¹H NMR (400 MHz, CDCl₃) δ ppm 1.63-1.76 (m, 2H), 1.95 (sept., 2H, J=10.3 Hz), 2.01-2.08 (m, 2H), 2.34 (dd, 1H, J=11.5 Hz, J=3.4 Hz), 2.61 (d, 1H, J=16.7 Hz), 2.73 (dd, 1H, J=11.5 Hz, J=3.3 Hz), 2.89 (quint., 1H, J=7.8 Hz), 3.12 (dd, 1H, J=16.9 Hz, J=3.9 Hz), 4.08 (br s, 1H), 5.83 (dq, 1H, J=9.9 Hz, J=1.8 Hz), 5.90 (dt, 1H, J=9.9 Hz, J=2.2 Hz); ¹³C NMR (100 MHz, CDCl₃) δ ppm 14.7, 27.1, 27.3, 27.8, 49.4, 54.3, 60.0, 64.3, 128.3, 128.4.

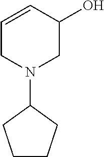 [SMILES: OC1C=CCN(C2CCCC2)C1]

Following general procedure E and starting from 1-cyclopentyl-1,2,3,6-tetrahydropyridin-3-yl pivalate (240 mg, 0.96 mmol, 1.0 equiv.), 1-cyclopentyl-1,2,3,6-tetrahydropyridin-3-ol was obtained as a yellow oil (98 mg, 0.59 mmol, 62%). ¹H NMR (400 MHz, CDCl₃) δ ppm 1.37-1.46 (m, 2H), 1.48-1.56 (m, 2H), 1.65-1.70 (m, 2H), 1.82-1.90 (m, 2H), 2.27 (br s, 1H), 2.48 (dd, 1H, J=11.5 Hz, J=3.1 Hz), 2.66 (quint., 1H, J=7.7 Hz), 2.77-2.84 (m, 1H), 3.19 (d, 1H, J=16.7 Hz), 4.06 (s, 1H), 5.83 (d, 1H, J=9.8 Hz), 5.88 (d, 1H, J=9.9 Hz); ¹³C NMR (100 MHz, CDCl₃) δ ppm 24.3, 24.5, 30.6, 30.8, 52.4, 56.8, 64.9, 66.9, 128.2, 129.4.

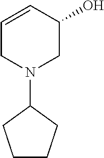 [SMILES: O[C@H]1C=CCN(C2CCCC2)C1]

Following general procedure E and starting from (S)-1-cyclopentyl-1,2,3,6-tetrahydropyridin-3-yl pivalate (99 mg, 0.39 mmol, 1.0 equiv.), (S)-1-cyclopentyl-1,2,3,6-tetrahydropyridin-3-ol was obtained as a yellow oil (40 mg, 0.24 mmol, 61%). ¹H NMR (400 MHz, CDCl₃) δ ppm 1.37-1.46 (m, 2H), 1.48-1.56 (m, 2H), 1.65-1.70 (m, 2H), 1.82-1.90 (m, 2H), 2.27 (br s, 1H), 2.48 (dd, 1H, J=11.5 Hz, J=3.1 Hz), 2.66 (quint., 1H, J=7.7 Hz), 2.77-2.84 (m, 1H), 3.19 (d, 1H, J=16.7 Hz), 4.06 (s, 1H), 5.83 (d, 1H, J=9.8 Hz), 5.88 (d, 1H, J=9.9 Hz); ¹³C NMR (100 MHz, CDCl₃) δ ppm 24.3, 24.5, 30.6, 30.8, 52.4, 56.8, 64.9, 66.9, 128.2, 129.4.

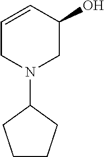 [SMILES: O[C@@H]1C=CCN(C2CCCC2)C1]

Following general procedure E and starting from (R)-1-cyclopentyl-1,2,3,6-tetrahydropyridin-3-yl pivalate (194 mg, 0.77 mmol, 1.0 equiv.), (R)-1-cyclopentyl-1,2,3,6-tetrahydropyridin-3-ol was obtained as a yellow oil (55 mg, 0.33 mmol, 43%). ¹H NMR (400 MHz, CDCl₃) δ ppm 1.37-1.46 (m, 2H), 1.48-1.56 (m, 2H), 1.65-1.70 (m, 2H), 1.82-1.90 (m, 2H), 2.27 (br s, 1H), 2.48 (dd, 1H, J=11.5 Hz, J=3.1 Hz), 2.66 (quint., 1H, J=7.7 Hz), 2.77-2.84 (m, 1H), 3.19 (d, 1H, J=16.7 Hz), 4.06 (s, 1H), 5.83 (d, 1H, J=9.8 Hz), 5.88 (d, 1H, J=9.9 Hz); ¹³C NMR (100 MHz, CDCl₃) δ ppm 24.3, 24.5, 30.6, 30.8, 52.4, 56.8, 64.9, 66.9, 128.2, 129.4.

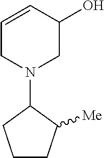 [SMILES: CC1CCCC1N1CC=CC(O)C1]

Following general procedure E and starting from 1-(2-methylcyclopentyl)-1,2,3,6-tetrahydropyridin-3-yl pivalate (152 mg, 0.57 mmol, 1.0 equiv.), 1-(2-methylcyclopentyl)-1,2,3,6-tetrahydropyridin-3-ol was obtained as a colorless oil (52 mg, 0.29 mmol, 50%). ¹H NMR (400 MHz, CDCl₃) δ ppm 0.82 (d, 3H, J=7.1 Hz), 1.39-1.60 (m, 3H), 1.68-1084 (m, 3H), 2.17-2.22 (m, 1H), 2.45 (dd, 1H, J=10.4 Hz, J=6.6 Hz), 2.52 (dd, 1H, J=11.4 Hz, J=3.1 Hz), 2.74 (d, 1H, J=16.0 Hz), 3.15 (d, 1H, J=16.0 Hz), 4.06 (br s, 1H), 5.81 (dq, 1H, J=9.8 Hz, J=2.7 Hz), 5.89 (dt, 1H, J=9.8 Hz, J=1.9 Hz); ¹³C NMR (100 MHz, CDCl₃) δ ppm 13.7, 20.5, 27.3, 31.7, 35.1, 53.1, 57.2, 64.8, 69.4, 128.4, 129.2.

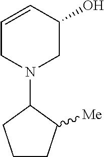 [SMILES: CC1CCCC1N1CC=C[C@H](O)C1]

Following general procedure E and starting from (S)-1-(2-methylcyclopentyl)-1,2,3,6-tetrahydropyridin-3-yl pivalate (215 mg, 0.81 mmol, 1.0 equiv.), (S)-1-(2-methylcyclopentyl)-1,2,3,6-tetrahydropyridin-3-ol was obtained as a colorless oil (47 mg, 0.26 mmol, 32%). ¹H NMR (400 MHz, CDCl₃) δ ppm 0.82 (d, 3H, J=7.1 Hz), 1.39-1.60 (m, 3H), 1.68-1084 (m, 3H), 2.17-2.22 (m, 1H), 2.45 (dd, 1H, J=10.4 Hz, J=6.6 Hz), 2.52 (dd, 1H, J=11.4 Hz, J=3.1 Hz), 2.74 (d, 1H, J=16.0 Hz), 3.15 (d, 1H, J=16.0 Hz), 4.06 (br s, 1H), 5.81 (dq, 1H, J=9.8 Hz, J=2.7 Hz), 5.89 (dt, 1H, J=9.8 Hz, J=1.9 Hz); ¹³C NMR (100 MHz, CDCl₃) δ ppm 13.7, 20.5, 27.3, 31.7, 35.1, 53.1, 57.2, 64.8, 69.4, 128.4, 129.2.

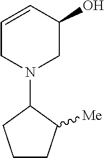 [SMILES: CC1CCCC1N1CC=C[C@@H](O)C1]

Following general procedure E and starting from (R)-1-(2-methylcyclopentyl)-1,2,3,6-tetrahydropyridin-3-yl pivalate (286 mg, 1.08 mmol, 1.0 equiv.), (R)-1-(2-methylcyclopentyl)-1,2,3,6-tetrahydropyridin-3-ol was obtained as a yellow oil (118 mg, 0.65 mmol, 61%). ¹H NMR (400 MHz, CDCl₃) δ ppm 0.82 (d, 3H, J=7.1 Hz), 1.39-1.60 (m, 3H), 1.68-1084 (m, 3H), 2.17-2.22 (m, 1H), 2.45 (dd, 1H, J=10.4 Hz, J=6.6 Hz), 2.52 (dd, 1H, J=11.4 Hz, J=3.1 Hz), 2.74 (d, 1H, J=16.0 Hz), 3.15 (d, 1H, J=16.0 Hz), 4.06 (br s, 1H), 5.81 (dq, 1H, J=9.8 Hz, J=2.7 Hz), 5.89 (dt, 1H, J=9.8 Hz, J=1.9 Hz); ¹³C NMR (100 MHz, CDCl₃) δ ppm 13.7, 20.5, 27.3, 31.7, 35.1, 53.1, 57.2, 64.8, 69.4, 128.4, 129.2.

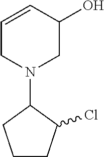 [SMILES: OC1C=CCN(C2CCCC2Cl)C1]

Following general procedure E and starting from 1-(2-chlorocyclopentyl)-1,2,3,6-tetrahydropyridin-3-yl pivalate (120 mg, 0.42 mmol, 1.0 equiv.), 1-(2-chlorocyclopentyl)-1,2,3,6-tetrahydropyridin-3-ol was obtained as a colorless oil (52 mg, 0.26 mmol, 61%). ¹H NMR (400 MHz, CDCl₃) δ ppm 1.29 (t, 1H, J=7.2 Hz), 1.66-1.82 (m, 2H), 1.89-1.99 (m, 2H), 2.05-2.10 (m, 2H), 2.62 (d, 1H, J=11.1 Hz), 2.81 (t, 2H, J=17.3 Hz), 3.28 (d, 1H, J=17.5 Hz), 4.07-4.13 (m, 1H), 4.42 (br s, 1H), 5.79 (d, 1H, J=9.8 Hz), 5.89 (d, 1H, J=8.6 Hz); ¹³C NMR (100 MHz, CDCl₃) δ ppm 20.1, 26.9, 34.4, 52.1, 56.6, 63.9, 64.4, 70.2, 128.1, 128.5.

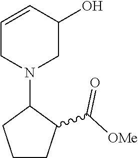 [SMILES: COC(=O)C1CCCC1N1CC=CC(O)C1]

Following general procedure E and starting from methyl 2-(3-(pivaloyloxy)-3,6-dihydropyridin-1(2H)-yl)cyclopentane-1-carboxylate (177 mg, 0.57 mmol, 1.0 equiv.), methyl 2-(3-hydroxy-3,6-dihydropyridin-1(2H)-yl)cyclopentane-1-carboxylate was obtained as a colorless oil (47 mg, 0.21 mmol, 36%). ¹H NMR (400 MHz, CDCl₃) δ ppm 1.71 (quint., 3H, J=7.0 Hz), 1.83 (sext., 2H, J=7.0 Hz), 1.94-2.00 (m, 3H), 2.53-2.58 (m, 1H), 2.83 (br s, 2H), 2.96 (d, 2H, J=16.7 Hz), 3.20 (d, 1H, J=13.3 Hz), 3.33-3.36 (m, 1H), 3.70 (s, 3H), 4.06 (br s, 1H), 5.81 (dq, 1H, J=9.9 Hz, J=2.2 Hz), 5.91 (d, 1H, J=9.9 Hz); ¹³C NMR (100 MHz, CDCl₃) δ ppm 14.5, 24.4, 28.9, 30.3, 46.9, 51.6, 52.2, 54.9, 61.0, 64.5, 69.8, 128.3.

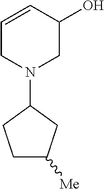 [SMILES: CC1CCC(N2CC=CC(O)C2)C1]

Following general procedure E and starting from 1-(3-methylcyclopentyl)-1,2,3,6-tetrahydropyridin-3-yl pivalate (200 mg, 0.75 mmol, 1.0 equiv.), 1-(3-methylcyclopentyl)-1,2,3,6-tetrahydropyridin-3-ol was obtained as a yellow oil (76 mg, 0.42 mmol, 56%). ¹H NMR (400 MHz, CDCl₃) δ ppm 0.97 (d, 1H, J=6.7 Hz), 1.01 (dd, 2H, J=6.4 Hz, J=1.6 Hz), 1.05-1.12 (m, 1H), 1.22-1.30 (m, 1H), 1.55-1.67 (m, 1H), 1.72-1.82 (m, 1H), 1.83-1.96 (m, 2H), 1.97-2.05 (m, 1H), 2.43-2.54 (m, 1H), 2.68-2.89 (m, 3H), 3.17-3.24 (m, 1H), 3.19 (br s, 1H), 4.08 (br s, 1H), 5.82 (d, 1H, J=9.9 Hz), 5.89 (d, 1H, J=9.8 Hz); ¹³C NMR (100 MHz, CDCl₃) δ ppm 21.0, 29.7, 32.7, 33.3, 39.9, 52.1, 56.6, 64.7, 66.9, 128.3, 128.9.

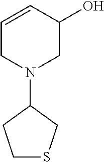 [SMILES: OC1C=CCN(C2CCSC2)C1]

Following general procedure E and starting from 1-(tetrahydrothiophen-3-yl)-1,2,3,6-tetrahydropyridin-3-yl pivalate (276 mg, 1.02 mmol, 1.0 equiv.), 1-(tetrahydrothiophen-3-yl)-1,2,3,6-tetrahydropyridin-3-ol was obtained as a brown oil (156 mg, 0.84 mmol, 82%). ¹H NMR (400 MHz, CD₃OD) δ ppm 1.83 (quint., 1H, J=9.0 Hz), 2.35 (sext., 1H, J=7.0 Hz), 2.47-2.54 (m, 1H), 2.72 (t, 1H, J=9.6 Hz), 2.84 (dd, 1H, J=9.1 Hz, J=4.2 Hz), 2.92-3.06 (m, 3H), 3.09-3.12 (m, 2H), 4.21 (br s, 1H), 5.76-5.84 (m, 2H); ¹³C NMR (100 MHz, CD₃OD) δ ppm 28.9, 33.0 (d, J=12.5 Hz), 33.9 (d, J=12.5 Hz), 52.2 (d, J=28.9 Hz), 57.0 (d, J=14.5 Hz), 65.7, 70.5, 128.3, 129.7.

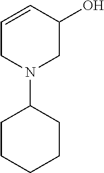 [SMILES: OC1C=CCN(C2CCCCC2)C1]

Following general procedure E and starting from 1-cyclohexyl-1,2,3,6-tetrahydropyridin-3-yl pivalate (191 mg, 0.72 mmol, 1.0 equiv.), 1-cyclohexyl-1,2,3,6-tetrahydropyridin-3-ol was obtained as a yellow oil (77 mg, 0.72 mmol, 59%). ¹H NMR (400 MHz, CDCl₃) δ ppm 1.08-1.13 (m, 1H), 1.19-1.33 (m, 4H), 1.64 (d, 1H, J=12.6 Hz), 1.77-1.82 (m, 3H), 1.89 (d, 1H, J=8.6 Hz), 2.41 (tt, 1H, J=10.7 Hz, J=3.1 Hz), 2.60 (dd, 1H, J=11.5 Hz, J=3.0 Hz), 2.84 (dd, 1H, J=11.5 Hz, J=3.3 Hz), 3.04 (d, 1H, J=16.9 Hz), 3.19 (dd, 1H, J=16.9 Hz, J=3.4 Hz), 4.05 (br s, 1H), 5.84 (dq, 1H, J=9.8 Hz, J=1.9 Hz), 5.89 (dt, 1H, J=9.8 Hz, J=2.1 Hz); ¹³C NMR (100 MHz, CDCl₃) δ ppm 26.1, 26.2, 26.6, 28.8, 29.3, 49.1, 53.9, 63.2, 64.9, 128.3, 129.6.

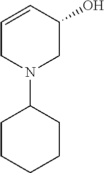 [SMILES: O[C@H]1C=CCN(C2CCCCC2)C1]

Following general procedure E and starting from (S)-1-cyclohexyl-1,2,3,6-tetrahydropyridin-3-yl pivalate (274 mg, 1.03 mmol, 1.0 equiv.), (S)-1-cyclohexyl-1,2,3,6-tetrahydropyridin-3-ol was obtained as a colorless oil (43 mg, 0.24 mmol, 23%). ¹H NMR (400 MHz, CDCl₃) δ ppm 1.08-1.13 (m, 1H), 1.19-1.33 (m, 4H), 1.64 (d, 1H, J=12.6 Hz), 1.77-1.82 (m, 3H), 1.89 (d, 1H, J=8.6 Hz), 2.41 (tt, 1H, J=10.7 Hz, J=3.1 Hz), 2.60 (dd, 1H, J=11.5 Hz, J=3.0 Hz), 2.84 (dd, 1H, J=11.5 Hz, J=3.3 Hz), 3.04 (d, 1H, J=16.9 Hz), 3.19 (dd, 1H, J=16.9 Hz, J=3.4 Hz), 4.05 (br s, 1H), 5.84 (dq, 1H, J=9.8 Hz, J=1.9 Hz), 5.89 (dt, 1H, J=9.8 Hz, J=2.1 Hz); ¹³C NMR (100 MHz, CDCl₃) δ ppm 26.1, 26.2, 26.6, 28.8, 29.3, 49.1, 53.9, 63.2, 64.9, 128.3, 129.6.

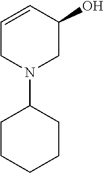 [SMILES: O[C@@H]1C=CCN(C2CCCCC2)C1]

Following general procedure E and starting from (R)-1-cyclohexyl-1,2,3,6-tetrahydropyridin-3-yl pivalate (246 mg, 0.93 mmol, 1.0 equiv.), (R)-1-cyclohexyl-1,2,3,6-tetrahydropyridin-3-ol was obtained as a colorless oil (84 mg, 0.46 mmol, 50%). ¹H NMR (400 MHz, CDCl₃) δ ppm 1.08-1.13 (m, 1H), 1.19-1.33 (m, 4H), 1.64 (d, 1H, J=12.6 Hz), 1.77-1.82 (m, 3H), 1.89 (d, 1H, J=8.6 Hz), 2.41 (tt, 1H, J=10.7 Hz, J=3.1 Hz), 2.60 (dd, 1H, J=11.5 Hz, J=3.0 Hz), 2.84 (dd, 1H, J=11.5 Hz, J=3.3 Hz), 3.04 (d, 1H, J=16.9 Hz), 3.19 (dd, 1H, J=16.9 Hz, J=3.4 Hz), 4.05 (br s, 1H), 5.84 (dq, 1H, J=9.8 Hz, J=1.9 Hz), 5.89 (dt, 1H, J=9.8 Hz, J=2.1 Hz); ¹³C NMR (100 MHz, CDCl₃) δ ppm 26.1, 26.2, 26.6, 28.8, 29.3, 49.1, 53.9, 63.2, 64.9, 128.3, 129.6.

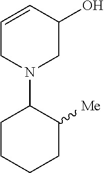 [SMILES: CC1CCCCC1N1CC=CC(O)C1]

Following general procedure E and starting from 1-(2-methylcyclohexyl)-1,2,3,6-tetrahydropyridin-3-yl pivalate (148 mg, 0.53 mmol, 1.0 equiv.), 1-(2-methylcyclohexyl)-1,2,3,6-tetrahydropyridin-3-ol was obtained as a colorless oil (50 mg, 0.26 mmol, 49%). ¹H NMR (400 MHz, CDCl₃) δ ppm 0.98 (br s, 3H), 1.20-1.28 (m, 2H), 1.38-1.49 (m, 4H), 1.62 (d, 1H, J=10.7 Hz), 1.79 (d, 2H, J=7.4 Hz), 2.29 (br s, 2H), 2.51 (br s, 1H), 2.86 (br s, 1H), 3.31 (br s, 1H), 4.07 (br s, 1H), 5.80-5.86 (m, 1H), 5.94 (br s, 1H); ¹³C NMR (100 MHz, CDCl₃) δ ppm 12.5, 20.1, 26.1, 26.2, 29.6, 30.3, 32.7, 32.8, 50.9, 54.3, 64.9, 127.9, 128.5.

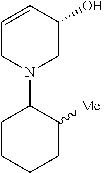 [SMILES: CC1CCCCC1N1CC=C[C@H](O)C1]

Following general procedure E and starting from (S)-1-(2-methylcyclohexyl)-1,2,3,6-tetrahydropyridin-3-yl pivalate (122 mg, 0.44 mmol, 1.0 equiv.), (S)-1-(2-methylcyclohexyl)-1,2,3,6-tetrahydropyridin-3-ol was obtained as a yellow oil (44 mg, 0.22 mmol, 51%). ¹H NMR (400 MHz, CDCl₃) δ ppm 0.98 (br s, 3H), 1.20-1.28 (m, 2H), 1.38-1.49 (m, 4H), 1.62 (d, 1H, J=10.7 Hz), 1.79 (d, 2H, J=7.4 Hz), 2.29 (br s, 2H), 2.51 (br s, 1H), 2.86 (br s, 1H), 3.31 (br s, 1H), 4.07 (br s, 1H), 5.80-5.86 (m, 1H), 5.94 (br s, 1H); ¹³C NMR (100 MHz, CDCl₃) δ ppm 12.5, 20.1, 26.1, 26.2, 29.6, 30.3, 32.7, 32.8, 50.9, 54.3, 64.9, 127.9, 128.5.

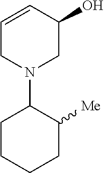 [SMILES: CC1CCCCC1N1CC=C[C@@H](O)C1]

Following general procedure E and starting from (R)-1-(2-methylcyclohexyl)-1,2,3,6-tetrahydropyridin-3-yl pivalate (102 mg, 0.36 mmol, 1.0 equiv.), (R)-1-(2-methylcyclohexyl)-1,2,3,6-tetrahydropyridin-3-ol was obtained as an orange oil (32 mg, 0.16 mmol, 45%). ¹H NMR (400 MHz, CDCl₃) δ ppm 0.98 (br s, 3H), 1.20-1.28 (m, 2H), 1.38-1.49 (m, 4H), 1.62 (d, 1H, J=10.7 Hz), 1.79 (d, 2H, J=7.4 Hz), 2.29 (br s, 2H), 2.51 (br s, 1H), 2.86 (br s, 1H), 3.31 (br s, 1H), 4.07 (br s, 1H), 5.80-5.86 (m, 1H), 5.94 (br s, 1H); ¹³C NMR (100 MHz, CDCl₃) δ ppm 12.5, 20.1, 26.1, 26.2, 29.6, 30.3, 32.7, 32.8, 50.9, 54.3, 64.9, 127.9, 128.5.

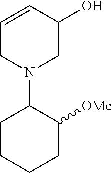 [SMILES: COC1CCCCC1N1CC=CC(O)C1]

Following general procedure E and starting from 1-(2-methoxycyclohexyl)-1,2,3,6-tetrahydropyridin-3-yl pivalate (103 mg, 0.35 mmol, 1.0 equiv.), 1-(2-methoxycyclohexyl)-1,2,3,6-tetrahydropyridin-3-ol was obtained as an orange oil (20 mg, 0.09 mmol, 27%). ¹H NMR (400 MHz, CDCl₃) δ ppm 1.11 (t, 1H, J=9.8 Hz), 1.34 (q, 1H, J=12.2 Hz), 1.72 (d, 2H, J=7.5 Hz), 1.96 (d, 1H, J=12.0 Hz), 2.16 (s, 1H), 2.18 (br s, 1H), 2.60 (t, 1H, J=10.2 Hz), 2.83 (d, 1H, J=11.5 Hz), 2.98 (d, 1H, J=11.5 Hz), 3.20 (t, 1H, J=9.8 Hz), 3.56 (d, 2H, J=16.7 Hz), 4.07 (br s, 1H), 5.82 (dq, 1H, J=9.9 Hz, J=1.9 Hz), 5.90 (dt, 1H, J=9.9 Hz, J=1.9 Hz); ¹³C NMR (100 MHz, CDCl₃) δ ppm 24.4, 25.3, 27.6, 30.7, 50.8, 53.7, 55.8, 64.4, 67.0, 80.7, 127.8, 129.1.

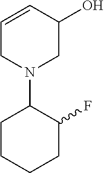 [SMILES: OC1C=CCN(C2CCCCC2F)C1]

Following general procedure E and starting from 1-(2-fluorocyclohexyl)-1,2,3,6-tetrahydropyridin-3-yl pivalate (122 mg, 0.43 mmol, 1.0 equiv.), 1-(2-fluorocyclohexyl)-1,2,3,6-tetrahydropyridin-3-ol was obtained as a yellow oil (19 mg, 0.09 mmol, 22%). ¹H NMR (400 MHz, CDCl₃) δ ppm 1.29-1.38 (m, 2H), 1.52 (t, 2H, J=13.8 Hz), 1.58-1.68 (m, 1H), 1.77 (td, 1H, J=13.5 Hz, J=3.4 Hz), 1.85 (d, 1H, J=14.3 Hz), 2.08 (d, 1H, J=10.4 Hz), 2.47 (dq, 1H, J=34.1 Hz, J=5.3 Hz), 2.63 (br s, 1H), 2.77 (td, 1H, J=12.1 Hz, J=2.7 Hz), 2.97 (ddd, 1H, J=27.8 Hz, J=11.8 Hz, J=2.6 Hz), 3.14-3.21 (m, 1H), 3.29 (d, 1H, J=16.9 Hz), 4.04 (br s, 1H), 5.09 (ddd, 1H, J=51.0 Hz, J=13.0 Hz, J=2.5 Hz), 5.83 (d, 1H, J=9.9 Hz), 5.90 (d, 1H, J=9.9 Hz); ¹⁹F NMR (376 MHz, CDCl₃) δ ppm −195.55; ¹³C NMR (100 MHz, CDCl₃) δ ppm 20.2, 23.2, 25.6 (d, J=12.0 Hz), 31.3 (d, J=21.7 Hz), 49.7 (d, J=13.5 Hz), 55.2 (d, J=21.7 Hz), 64.8, 65.1 (dd, J=18.3 Hz, J=6.7 Hz), 90.8 (dd, J=174.8 Hz, J=56.8 Hz), 128.1, 129.4.

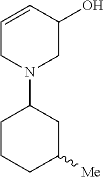 [SMILES: CC1CCCC(N2CC=CC(O)C2)C1]

Following general procedure E and starting from 1-(3-methylcyclohexyl)-1,2,3,6-tetrahydropyridin-3-yl pivalate (65 mg, 0.23 mmol, 1.0 equiv.), 1-(3-methylcyclohexyl)-1,2,3,6-tetrahydropyridin-3-ol was obtained as a yellow oil (28 mg, 0.14 mmol, 61%). ¹H NMR (400 MHz, CDCl₃) δ ppm 0.93 (d, 3H, J=7.1 Hz), 1.21-1.27 (m, 1H), 1.43-1.73 (m, 7H), 1.98-2.01 (m, 1H), 2.51 (t, 1H, J=11.2 Hz), 2.63 (br s, 1H), 2.92 (d, 1H, J=16.5 Hz), 3.01 (d, 1H, J=11.3 Hz), 3.31 (d, 1H, J=16.9 Hz), 4.07 (br s, 1H), 5.84 (dq, 1H, J=9.8 Hz, J=2.2 Hz), 5.94 (d, 1H, J=9.7 Hz).

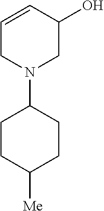 [SMILES: CC1CCC(N2CC=CC(O)C2)CC1]

Following general procedure E and starting from 1-(4-methylcyclohexyl)-1,2,3,6-tetrahydropyridin-3-yl pivalate (58 mg, 0.21 mmol, 1.0 equiv.), 1-(4-methylcyclohexyl)-1,2,3,6-tetrahydropyridin-3-ol was obtained as a yellow oil (24 mg, 0.12 mmol, 24%). ¹H NMR (400 MHz, CDCl₃) δ ppm 0.92 (d, 3H, J=6.9 Hz), 1.48 (q, 4H, J=5.4 Hz), 1.53-1.59 (m 2H), 1.64-1.77 (m, 3H), 2.37 (sept., 1H, J=3.4 Hz), 2.50 (dd, 1H, J=11.7 Hz, J=3.0 Hz), 2.91 (dd, 1H, J=16.8 Hz, J=1.4 Hz), 2.97 (dd, 1H, J=11.5 Hz, J=3.1 Hz), 3.28 (dd, 1H, J=16.8 Hz, J=4.1 Hz), 4.06 (q, 1H, J=2.4 Hz), 5.85 (dq, 1H, J=9.8 Hz, J=2.2 Hz), 5.92 (dt, 1H, J=9.8 Hz, J=2.2 Hz); ¹³C NMR (100 MHz, CDCl₃) δ ppm 19.6, 24.9, 25.2, 27.9, 30.7, 30.8, 49.7, 54.3, 61.6, 64.8, 128.2, 129.3.

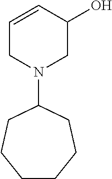 [SMILES: OC1C=CCN(C2CCCCCC2)C1]

Following general procedure E and starting from 1-cycloheptyl-1,2,3,6-tetrahydropyridin-3-yl pivalate (89 mg, 0.32 mmol, 1.0 equiv.), 1-cycloheptyl-1,2,3,6-tetrahydropyridin-3-ol was obtained as an orange oil (51 mg, 0.26 mmol, 82%). ¹H NMR (400 MHz, CDCl₃) δ ppm 1.41-1.61 (m, 6H), 1.69-1.76 (m, 2H), 1.93-2.00 (m, 2H), 2.75 (d, 1H, J=11.5 Hz), 2.84 (t, 1H, J=9.3 Hz), 2.97 (d, 1H, J=11.3 Hz), 3.16 (d, 1H, J=16.5 Hz), 3.31 (d, 1H, J=15.0 Hz), 4.11 (br s, 1H), 5.84 (d, 1H, J=9.9 Hz), 5.95 (d, 1H, J=9.9 Hz); ¹³C NMR (100 MHz, CDCl₃) δ ppm 25.6, 25.7, 28.1, 28.2, 29.3, 30.5, 48.3, 53.4, 64.1, 65.3, 127.6, 128.4.

### Preparation of 1-propyl-1,2,3,6-tetrahydropyridin-3-ol

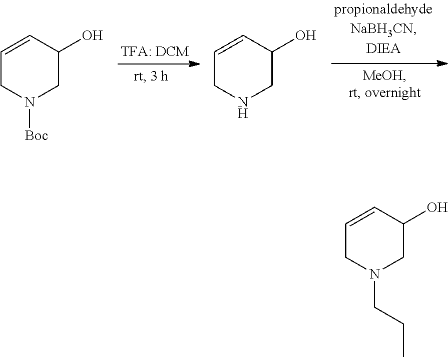

### Step 1. Formation of 1,2,3,6-tetrahydropyridin-3-ol 2,2,2-trifluoroacetate

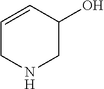

tert-Butyl 3-hydroxy-3,6-dihydropyridine-1(2H)-carboxylate (540 mg, 2.70 mmol, 1.0 equiv.) was dissolved in DCM (7 mL, 0.38 M) and cooled down at 0° C. TFA (3.4 mL, 43.50 mmol, 16.0 equiv.) was added dropwise and the resulting mixture was stirred at room temperature for 3 hours. The reaction mixture was concentrated under vacuum, to afford 1,2,3,6-tetrahydropyridin-3-ol 2,2,2-trifluoroacetate as a brown oil (576 mg, 2.70 mmol, 100%).

### Step 2. Formation of 1-propyl-1,2,3,6-tetrahydropyridin-3-ol

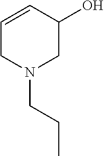

In an oven-dried round-bottom flask under argon, 1,2,3,6-tetrahydropyridin-3-ol 2,2,2-trifluoroacetate (576 mg, 2.70 mmol, 1.0 equiv.) was dissolved in anhydrous methanol (15 mL, 0.18 M) and cooled down at 0° C. Propionaldehyde (581 μL, 8.11 mmol, 3.0 equiv.) and DIEA (1.9 mL, 10.81 mmol, 4.0 equiv.) were added and stirred at 0° C. for 30 minutes. NaBH₃CN (425 mg, 6.76 mmol, 2.5 equiv.) was added and the resulting reaction mixture was stirred at room temperature overnight. The reaction mixture was concentrated under vacuum. The residue was resuspended in water and the aqueous phase was extracted 3 times with EtOAc. The combined organic layers were washed with brine, dried over MgSO₄ and concentrated under vacuum. The crude was purified by chromatography on silica gel (EtOAc:methanol 1:0 to 8:2), to afford 1-propyl-1,2,3,6-tetrahydropyridin-3-ol as an orange oil (95 mg, 0.67 mmol, 25%). ¹H NMR (400 MHz, CDCl₃) δ ppm 0.94 (t, 3H, J=7.4 Hz), 1.55 (q, 2H, J=7.4 Hz), 2.40-2.52 (m, 3H), 2.75-2.84 (m, 3H), 3.19 (dd, 1H, J=17.0 Hz, J=4.1 Hz), 4.08 (br s, 1H), 5.84 (dq, 1H, J=9.8 Hz, J=2.2 Hz), 5.91 (dt, 1H, J=9.9 Hz, J=2.2 Hz); ¹³C NMR (100 MHz, CDCl₃) δ ppm 12.1, 20.2, 53.1, 58.1, 60.2, 64.7, 128.3, 128.9.

Preparation of various compounds

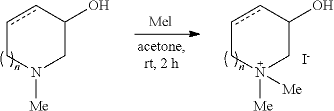

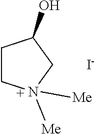

(R)-1-Methylpyrrolidin-3-ol (51 mg, 0.50 mmol, 1.0 equiv.) was dissolved in acetone (2 mL, 0.25 M). The resulting mixture was bubbled with argon for 10 minutes and Mel (37.0 μL, 0.60 mmol, 1.2 equiv.) was added dropwise. The reaction mixture was stirred at room temperature for 2 hours. The formed precipitate was filtrated under vacuum, to afford (R)-3-hydroxy-1,1-dimethylpyrrolidin-1-ium iodide as a grey solid (121 mg, 0.50 mmol, 100%). ¹H NMR (500 MHz, CD₃OD) δ ppm 2.15-2.22 (m, 1H), 2.59 (sext., 1H, J=6.8 Hz), 3.21 (s, 3H), 3.33 (s, 3H), 3.56 (d, 1H, J=12.9 Hz), 3.59-3.65 (m, 1H), 3.67 (dd, 1H, J=12.7 Hz, J=5.4 Hz), 3.82 (dt, 1H, J=11.7 Hz, J=7.8 Hz), 4.67 (br s, 1H).

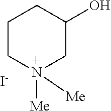

1-Methylpiperidin-3-ol (55 mg, 0.48 mmol, 1.0 equiv.) was dissolved in acetone (2 mL, 0.25 M). The resulting mixture was bubbled with argon for 10 minutes and Mel (37.0 μL, 0.60 mmol, 1.2 equiv.) was added dropwise. The reaction mixture was stirred at room temperature for 2 hours. The formed precipitate was filtrated under vacuum, to afford (3-hydroxy-1,1-dimethylpiperidin-1-ium iodide as a grey solid (91 mg, 0.35 mmol, 73%). ¹H NMR (500 MHz, CD₃OD) δ ppm 1.64 (m, 1H), 1.88-1.94 (m, 2H), 2.11-2.17 (m, 1H), 3.15 (s, 3H), 3.25 (dd, 1H, J=12.7 Hz, J=6.1 Hz), 3.29 (s, 3H), 3.39-3.42 (m, 2H), 3.46 (dd, 1H, J=12.9 Hz, J=3.4 Hz), 4.14 (br s, 1H).

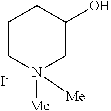

1-Methyl-1,2,3,6-tetrahydropyridin-3-ol (53 mg, 0.47 mmol, 1.0 equiv.) was dissolved in acetone (1.8 mL, 0.25 M). The resulting mixture was bubbled with argon for 10 minutes and Mel (35.0 μL, 0.56 mmol, 1.2 equiv.) was added dropwise. The reaction mixture was stirred at room temperature for 3 hours. The formed precipitate was filtrated under vacuum, to afford 3-hydroxy-1,1-dimethyl-1,2,3,6-tetrahydropyridin-1-ium iodide as a white solid (68 mg, 0.27 mmol, 57%). ¹H NMR (400 MHz, CD₃OD) δ ppm 2.59 (s, 3H), 2.62 (s, 3H), 2.64-2.67 (m, 2H), 2.85 (dd, 1H, J=12.9 Hz, J=3.9 Hz), 3.06 (dd, 1H, J=13.0 Hz, J=5.1 Hz), 3.84 (br s, 1H), 5.23 (d, 1H, J=10.5 Hz), 5.47 (dt, 1H, J=10.4 Hz, J=1.7 Hz); ¹³C NMR (100 MHz, CD₃OD) δ ppm 53.2, 54.3, 61.6, 61.8, 66.0, 121.3, 129.2.

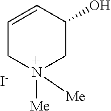

(S)-1-Methyl-1,2,3,6-tetrahydropyridin-3-ol (17 mg, 0.15 mmol, 1.0 equiv.) was dissolved in acetone (0.6 mL, 0.25 M). The resulting mixture was bubbled with argon for 10 minutes and Mel (14.0 μL, 0.23 mmol, 1.5 equiv.) was added dropwise. The reaction mixture was stirred at room temperature for 3 hours. The formed precipitate was filtrated under vacuum, to afford (S)-3-hydroxy-1,1-dimethyl-1,2,3,6-tetrahydropyridin-1-ium iodide as a white solid (29 mg, 0.11 mmol, 75%). ¹H NMR (400 MHz, CD₃OD) δ ppm 2.92 (s, 3H), 2.96 (s, 3H), 3.18 (dd, 1H, J=13.0 Hz, J=3.9 Hz), 3.38 (dd, 1H, J=12.9 Hz, J=5.0 Hz), 3.64-3.67 (m, 2H), 4.16 (br s, 1H), 5.57 (d, 1H, J=10.4 Hz), 5.81 (dt, 1H, J=10.5 Hz, J=1.6 Hz).

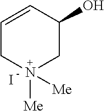

(R)-1-Methyl-1,2,3,6-tetrahydropyridin-3-ol (120 mg, 1.06 mmol, 1.0 equiv.) was dissolved in acetone (4.2 mL, 0.25 M). The resulting mixture was bubbled with argon for 10 minutes and Mel (99.0 μL, 1.59 mmol, 1.5 equiv.) was added dropwise. The reaction mixture was stirred at room temperature for 4 hours. The formed precipitate was filtrated under vacuum, to afford (R)-3-hydroxy-1,1-dimethyl-1,2,3,6-tetrahydropyridin-1-ium iodide as a white solid (213 mg, 0.84 mmol, 79%). ¹H NMR (400 MHz, CD₃OD) δ ppm 3.25 (s, 3H), 3.28 (s, 3H), 3.50 (dd, 1H, J=13.0 Hz, J=4.0 Hz), 3.72 (dd, 1H, J=13.0 Hz, J=5.1 Hz), 3.98-4.02 (m, 2H), 4.50 (br s, 1H), 5.88 (d, 1H, J=10.3 Hz), 6.12 (dt, 1H, J=10.5 Hz, J=1.3 Hz); ¹³C NMR (100 MHz, CD₃OD) δ ppm 53.2, 54.3, 61.6, 61.8, 66.0, 121.3, 129.2.

### Preparation of 1-methyl-1,2,3,6-tetrahydropyridin-3-d-3-ol

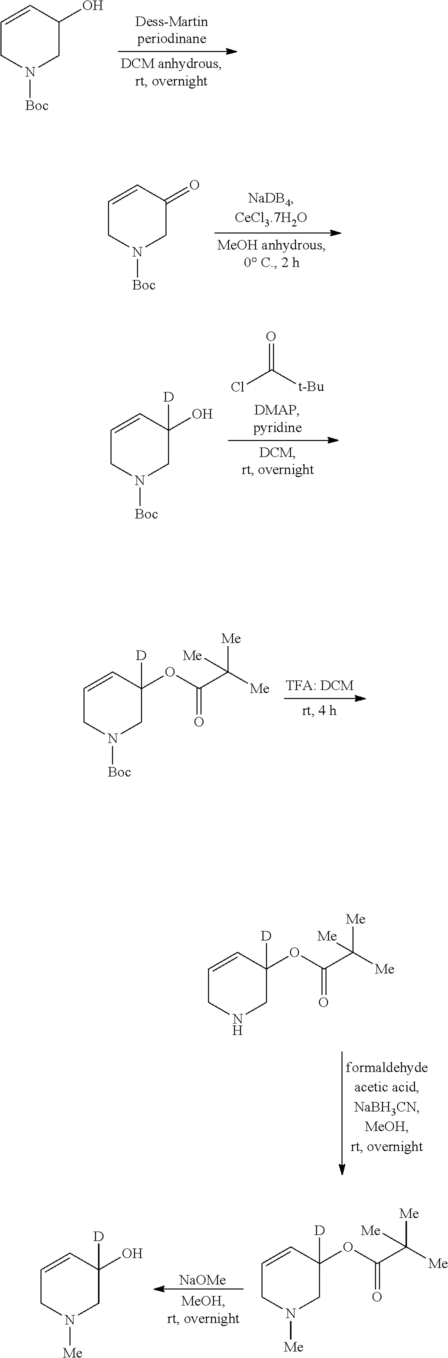

### Step 1. Formation of tert-butyl 3-oxo-3,6-dihydropyridine-1(2H)-carboxylate

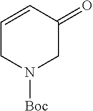 [SMILES: CC(C)(C)OC(=O)N1CC=CC(=O)C1]

tert-Butyl 3-hydroxy-3,6-dihydropyridine-1(2H)-carboxylate (1.0 g, 5.02 mmol, 1.0 equiv). was dissolved in anhydrous DCM (20 mL, 0.25 M) and the Dess-Martin periodinane (2.4 g, 5.77 mmol, 1.2 equiv.) was added. The resulting mixture was stirred at room temperature overnight. The reaction mixture was treated with a 20% solution of Na₂S₂O₃, a saturated solution of NaHCO₃ and brine, dried over MgSO₄ and concentrated under vacuum. The crude was purified by chromatography on silica gel (hexanes:EtOAc 2:1 to 1:1), to tert-butyl 3-oxo-3,6-dihydropyridine-1(2H)-carboxylate as an orange oil (555 mg, 2.81 mmol, 56%). ¹H NMR (400 MHz, CDCl₃) δ ppm 1.47 (s, 9H), 4.11 (s, 2H), 4.23 (t, 2H, J=2.4 Hz), 6.17 (dt, 1H, J=10.3 Hz, J=2.2 Hz), 7.02 (br s, 1H); ¹³C NMR (100 MHz, CDCl₃) δ ppm 28.6, 42.9, 81.4, 127.9, 147.7, 154.5, 193.7.

### Step 2. Formation of tert-butyl 3-hydroxy-3,6-dihydropyridine-1(2H)-carboxylate-3-d

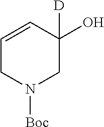 [SMILES: [2H]C1(O)C=CCN(C(=O)OC(C)(C)C)C1]

In an oven dried round-bottom flask under argon, tert-butyl 3-oxo-3,6-dihydropyridine-1(2H)-carboxylate (400 mg, 2.03 mmol, 1.0 equiv.) and CeCl₃.7H₂O (756 mg, 2.03 mmol, 1.0 equiv.) were dissolved in anhydrous methanol (20 mL, 0.1 M) and cooled down at 0° C. NaBD₄ (212 mg, 5.07 mmol, 2.5 equiv.) was added portion wise and the resulting mixture was stirred at 0° C. for 2 hours. The reaction was quenched with water and concentrated under vacuum. The residue was diluted in water and extracted 3 times with EtOAc. The combined organic layers were washed with brine, dried over MgSO₄ and concentrated under vacuum, to afford tert-butyl 3-hydroxy-3,6-dihydropyridine-1(2H)-carboxylate-3-d as a colorless oil (345 mg, 1.72 mg, 85%). ¹H NMR (400 MHz, CDCl₃) δ ppm 1.47 (s, 9H), 3.48-3.57 (m, 2H), 3.79 (d, 1H, J=18.8 Hz), 3.97 (d, 1H, J=17.6 Hz), 5.82 (d, 1H, J=10.1 Hz), 5.90 (d, 1H, J=10.1 Hz); ¹³C NMR (100 MHz, CDCl₃) δ ppm 28.7, 43.6 (d, J=47.2 Hz), 47.9 (d, J=103.1 Hz), 63.7 (t, J=22.2 Hz), 80.4, 128.4, 128.5, 155.6.

### Step 3. Formation of tert-butyl 3-(pivaloyloxy)-3,6-dihydropyridine-1(2H)-carboxylate-3-d

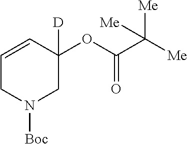 [SMILES: [2H]C1(OC(=O)C(C)(C)C)C=CCN(C(=O)OC(C)(C)C)C1]

tert-Butyl allyl(2-hydroxybut-3-en-1-yl)carbamate-3-d (343 mg, 1.71 mmol, 1.0 equiv.) and DMAP (21 mg, 0.17 mmol, 0.1 equiv.) were dissolved in anhydrous DCM (5.7 mL, 0.3 M). Pyridine (693 μL, 8.56 mmol, 5.0 equiv.) and pivaloyl chloride (295 μL, 2.40 mmol, 1.4 equiv.) were added and the resulting mixture was stirred at room temperature overnight. The reaction mixture was diluted with water. The aqueous phase was extracted 3 times with DCM. The combined organic layers were washed with brine, dried over MgSO₄ and concentrated under vacuum. The crude was purified by chromatography on silica gel (hexanes:EtOAc 4:1), to obtain tert-butyl 3-(pivaloyloxy)-3,6-dihydropyridine-1(2H)-carboxylate-3-d as a colorless oil (413 mg, 1.45 mmol, 85%). ¹H NMR (400 MHz, CDCl₃) δ ppm 1.18 (s, 9H), 1.46 (s, 9H), 3.44 (d, 1H, J=13.7 Hz), 3.73-3.80 (m, 2H), 4.09-4.15 (m, 1H), 5.84 (d, 1H, J=9.6 Hz), 5.97 (br s, 1H); ¹³C NMR (100 MHz, CDCl₃) δ ppm 26.8, 27.4, 28.7, 39.1, 42.8 (t, J=142.1 Hz), 60.7, 80.3, 124.7, 130.6, 155.0, 178.4.

### Step 4. Formation of 1,2,3,6-tetrahydropyridin-3-yl-3-d pivalate

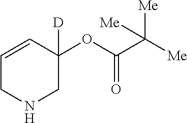 [SMILES: [2H]C1(OC(=O)C(C)(C)C)C=CCNC1]

Following general procedure B and starting from tert-butyl 3-(pivaloyloxy)-3,6-dihydropyridine-1(2H)-carboxylate-3-d (413 mg, 1.45 mmol, 1.0 equiv.), 1,2,3,6-tetrahydropyridin-3-yl-3-d pivalate was obtained as a brown oil (268 mg, 1.45 mmol, 100%).

### Step 5. Formation of 1-methyl-1,2,3,6-tetrahydropyridin-3-yl-3-d pivalate

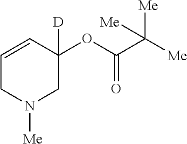 [SMILES: [2H]C1(OC(=O)C(C)(C)C)C=CCN(C)C1]

Following general procedure C and starting from 1,2,3,6-tetrahydropyridin-3-yl-3-d pivalate (268 mg, 1.45 mmol, 1.0 equiv.) and formaldehyde (37% in water, 325 μL, 4.36 mmol, 3.0 equiv.), 1-methyl-1,2,3,6-tetrahydropyridin-3-yl-3-d pivalate was obtained as a colorless oil (124 mg, 0.62 mmol, 43%). ¹H NMR (400 MHz, CDCl₃) δ ppm 1.20 (s, 9H), 2.35 (s, 3H), 2.46 (d, 1H, J=11.8 Hz), 2.76 (d, 1H, J=11.7 Hz), 2.87 (dt, 1H, J=16.8 Hz, J=2.6 Hz), 2.98 (dt, 1H, J=16.8 Hz, J=2.6 Hz), 5.72 (dt, 1H, J=10.1 Hz, J=1.8 Hz), 5.95 (dt, 1H, J=9.9 Hz, J=3.3 Hz); ¹³C NMR (100 MHz, CDCl₃) δ ppm 27.5, 39.0, 45.9, 54.3, 56.6, 67.2 (t, J=23.1 Hz), 124.4, 130.5, 178.7.

### Step 6. Formation of 1-methyl-1,2,3,6-tetrahydropyridin-3-d-3-ol

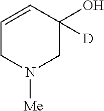

Following general procedure E and starting from 1-methyl-1,2,3,6-tetrahydropyridin-3-yl-3-d pivalate (111 mg, 0.56 mmol, 1.0 equiv.), 1-methyl-1,2,3,6-tetrahydropyridin-3-d-3-ol was obtained as a colorless oil (36 mg, 0.31 mmol, 56%). ¹H NMR (400 MHz, CDCl₃) δ ppm 2.37 (s, 3H), 2.51 (d, 1H, J=11.8 Hz), 2.71 (dt, 1H, J=16.7 Hz, J=2.1 Hz), 2.78 (d, 1H, J=11.8 Hz), 3.15 (dd, 1H, J=16.7 Hz, J=4.0 Hz), 4.03 (br s, 1H), 5.82 (dq, 1H, J=10.1 Hz, J=2.2 Hz), 5.90 (d, 1H, J=9.9 Hz); ¹³C NMR (100 MHz, CDCl₃) δ ppm 45.7, 54.4, 60.2, 64.2 (t, J=22.2 Hz), 127.9, 128.2.

### Preparation of 1,5-dimethyl-1,2,3,6-tetrahydropyridin-3-ol

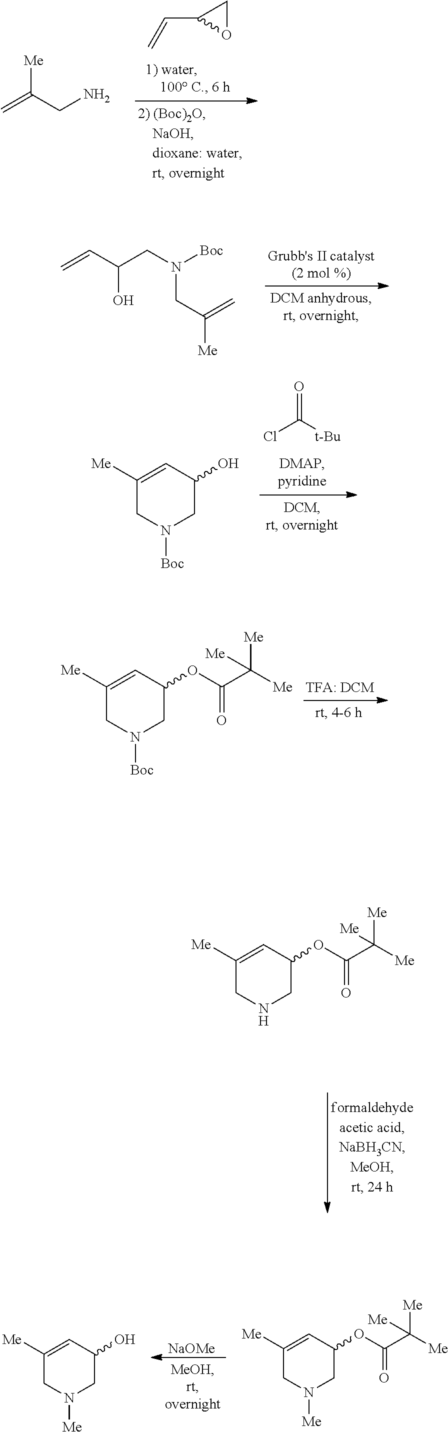

### Step 1. Formation of tert-butyl (2-hydroxybut-3-en-1-yl)(2-methylallyl)carbamate

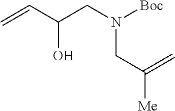 [SMILES: C=CC(O)CN(CC(=C)C)C(=O)OC(C)(C)C]

To a solution of 2-methylprop-2-en-1-amine (1.0 mL, 11.24 mmol, 3.0 equiv) and water (54 μL, 2.98 mmol, 0.8 equiv) was added 2-vinyloxirane (300 μL, 3.72 mmol, 1.0 equiv.). The mixture was heated at 80° C. for 6 hours. Once cooled down, the reaction mixture was concentrated under vacuum. The residue was dissolved in a mixture dioxane:water (1:1, 10 mL, 0.37 M). A 50% solution of NaOH (393 μL, 7.45 mmol, 2.0 equiv.) and di-tert-butyl decarbonate (1.6 g, 7.45 mmol, 2.0 equiv.) were added and the resulting mixture was stirred at room temperature overnight. The reaction mixture was diluted with EtOAc and washed twice with a 20% citric acid solution and then brine. The organic layer was dried over MgSO₄ and concentrated under vacuum, to afford tert-butyl (2-hydroxybut-3-en-1-yl)(2-methylallyl)carbamate as a colorless oil (899 mg, 3.72 mmol, 100%). ¹H NMR (400 MHz, CDCl₃) δ ppm 1.46 (s, 9H), 1.68 (s, 3H), 3.21-3.36 (m, 1H), 3.70 (s, 2H), 2.78-3.83 (m, 2H), 4.33 (q, 1H, J=5.4 Hz), 4.76 (s, 1H), 5.14-5.23 (m, 1H), 5.33 (d, 1H, J=16.9 Hz), 5.78-5.87 (m, 1H).

### Step 2. Formation of tert-butyl 3-hydroxy-5-methyl-3,6-dihydropyridine-1(2H)-carboxylate

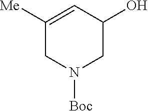 [SMILES: CC1=CC(O)CN(C(=O)OC(C)(C)C)C1]

Following general procedure A and starting from tert-butyl (2-hydroxybut-3-en-1-yl)(2-methylallyl)carbamate (899 mg, 3.73 mmol, 1.0 equiv.), tert-butyl 3-hydroxy-5-methyl-3,6-dihydropyridine-1(2H)-carboxylate was obtained as a brown oil (585 mg, 2.74 mmol, 74%). ¹H NMR (400 MHz, CDCl₃) δ ppm 1.48 (s, 9H), 1.72 (s, 3H), 3.42 (d, 1H, J=12.1 Hz), 3.55-3.65 (m, 2H), 3.89 (br s, 1H), 5.64 (br s, 1H).

### Step 3. Formation of tert-butyl 5-methyl-3-(pivaloyloxy)-3,6-dihydropyridine-1(2H)-carboxylate

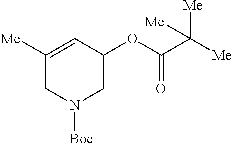 [SMILES: CC1=CC(OC(=O)C(C)(C)C)CN(C(=O)OC(C)(C)C)C1]

tert-Butyl 3-hydroxy-5-methyl-3,6-dihydropyridine-1(2H)-carboxylate (560 mg, 263 mmol, 1.0 equiv.) and DMAP (64 mg, 0.53 mmol, 0.1 equiv.) were dissolved in anhydrous DCM (8.7 mL, 0.3 M). Pyridine (1.0 mL, 13.13 mmol, 5.0 equiv.) and pivaloyl chloride (485 μL, 3.94 mmol, 1.5 equiv.) were added and the resulting mixture was stirred at room temperature overnight. The reaction mixture was diluted with water. The aqueous phase was extracted 3 times with DCM. The combined organic layers were washed with brine, dried over MgSO₄ and concentrated under vacuum. The crude was purified by chromatography on silica gel (hexanes:EtOAc 4:1), to obtain tert-butyl 5-methyl-3-(pivaloyloxy)-3,6-dihydropyridine-1(2H)-carboxylate as a colorless oil (636 mg, 2.14 mmol, 81%). ¹H NMR (400 MHz, CDCl₃) δ ppm 1.18 (s, 9H), 1.46 (s, 9H), 1.74 (s, 3H), 3.30 (dd, 1H, J=14.0 Hz, J=3.5 Hz), 3.57 (d, 1H, J=18.0 Hz), 3.84 (dd, 1H, J=13.8 Hz, J=3.7 Hz), 4.07-4.13 (m, 1H), 5.11 (br s, 1H), 5.57 (br s, 1H).

### Step 4. Formation of 5-methyl-1,2,3,6-tetrahydropyridin-3-yl pivalate

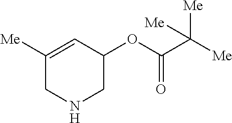 [SMILES: CC1=CC(OC(=O)C(C)(C)C)CNC1]

Following general procedure B and starting from tert-butyl 5-methyl-3-(pivaloyloxy)-3,6-dihydropyridine-1(2H)-carboxylate (635 mg, 2.14 mmol, 1.0 equiv.), 5-methyl-1,2,3,6-tetrahydropyridin-3-yl pivalate was obtained as a brown oil (320 mg, 1.62 mmol, 76%).

### Step 5. Formation of 1,5-dimethyl-1,2,3,6-tetrahydropyridin-3-yl pivalate

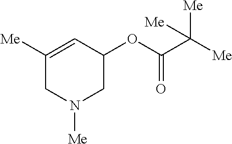 [SMILES: CC1=CC(OC(=O)C(C)(C)C)CN(C)C1]

Following general procedure C and starting from 5-methyl-1,2,3,6-tetrahydropyridin-3-yl pivalate (320 mg, 1.62 mmol, 1.0 equiv.) and formaldehyde (37% in water, 362 μL, 4.87 mmol, 3.0 equiv.), 1,5-dimethyl-1,2,3,6-tetrahydropyridin-3-yl pivalate was obtained as an orange oil (116 mg, 0.55 mmol, 34%). ¹H NMR (400 MHz, CDCl₃) δ ppm 1.20 (s, 9H), 1.72 (s, 3H), 2.35 (s, 3H), 2.46 (dd, 1H, J=4.8 Hz, J=11.8 Hz), 2.66-2.75 (m, 2H), 2.89 (d, 1H, J=16.1 Hz), 5.26 (br s, 1H), 5.45 (br s, 1H).

### Step 6. Formation of 1,5-dimethyl-1,2,3,6-tetrahydropyridin-3-ol

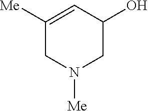

Following general procedure E and starting from 1,5-dimethyl-1,2,3,6-tetrahydropyridin-3-yl pivalate (116 mg, 0.55 mmol, 1.0 equiv.), 1,5-dimethyl-1,2,3,6-tetrahydropyridin-3-ol was obtained as a colorless oil (38 mg, 0.30 mmol, 55%). ¹H NMR (400 MHz, CDCl₃) δ ppm 2.35 (s, 3H), 2.38 (dd, 1H, J=11.4 Hz, J=3.3 Hz), 2.57 (d, 1H, J=16.1 Hz), 2.67 (dd, 1H, J=11.4 Hz, J=3.2 Hz), 2.95 (d, 1H, J=16.1 Hz), 4.04 (d, 1H, J=2.9 Hz), 5.60 (d, 1H, J=2.3 Hz); ¹³C NMR (100 MHz, CDCl₃) δ ppm 21.0, 45.9, 59.2, 60.1, 65.2, 122.7, 137.2.

### Preparation of 1-methyl-1,2,3,6-tetrahydropyridin-3-yl acetate

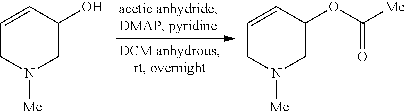 [SMILES: CN1CC=CC(O)C1 ; CC(=O)OC1C=CCN(C)C1]

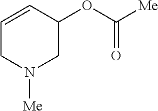 [SMILES: CC(=O)OC1C=CCN(C)C1]

1-Methyl-1,2,3,6-tetrahydropyridin-3-ol (46 mg, 0.41 mmol, 1.0 equiv.) and DMAP (10 mg, 0.08 mmol, 0.2 equiv.) was dissolved in pyridine (164 μL, 2.03 mmol, 5.0 equiv.) and anhydrous DCM (1.3 mL, 0.3 M). Acetic anhydride (57.5 μL, 0.61 mmol, 1.5 equiv.) was added dropwise and the resulting mixture was stirred at room temperature overnight. The reaction mixture was diluted with water and the aqueous phase was extracted 3 times with EtOAc. The combined organic layers were washed with brine, dried over MgSO₄ and concentrated under vacuum. The crude was purified by chromatography on silica gel (hexanes:EtOAc 1:1), to obtain 1-methyl-1,2,3,6-tetrahydropyridin-3-yl acetate as a yellow oil (17 mg, 0.11 mmol, 28%). ¹H NMR (400 MHz, CDCl₃) δ ppm 2.68 (s, 3H), 2.97 (s, 3H), 3.20 (dd, 1H, J=12.2 Hz, J=4.0 Hz), 3.36 (d, 1H, J=15.9 Hz), 3.78 (d, 1H, J=16.8 Hz), 5.86 (br s, 1H), 6.41 (dq, 1H, J=10.1 Hz, J=1.8 z), 6.60 (dt, 1H, J=9.9 Hz, J=3.0 Hz); ¹³C NMR (100 MHz, CDCl₃) δ ppm 21.6, 45.8, 54.1, 56.7, 67.5, 123.5, 131.3, 171.2.

### Preparation of Various Compounds

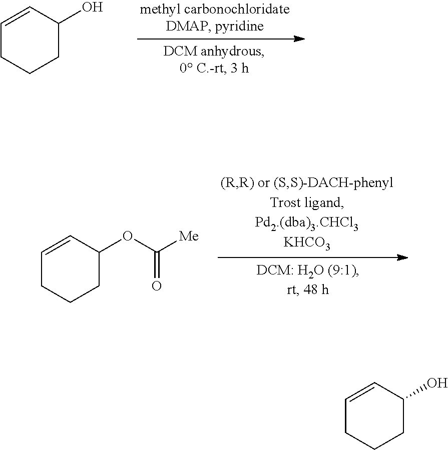 [SMILES: OC1C=CCCC1 ; CC(=O)OC1C=CCCC1 ; O[C@H]1C=CCCC1]

### Step 1. Formation of cyclohex-2-en-1-yl methyl carbonate

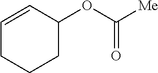

Cyclohex-2-en-1-ol (300 μL, 3.04 mmol, 1.0 equiv.) and DMAP (74 mg, 0.61 mmol, 0.2 equiv.) was dissolved in pyridine (10 mL, 124 mmol, 40.7 equiv.) and anhydrous DCM (5 mL). Methyl carbonochloridate (586 μL, 7.59 mmol, 2.5 equiv.) was added dropwise and the resulting mixture was stirred at room temperature for 3 hours. The reaction mixture was diluted with water and the aqueous phase was extracted 3 times with EtOAc. The combined organic layers were washed with brine, dried over MgSO₄ and concentrated under vacuum. The crude was purified by chromatography on silica gel (hexanes:EtOAc 8:1), to obtain cyclohex-2-en-1-yl methyl carbonate as a colorless oil (317 mg, 2.03 mmol, 67%). ¹H NMR (400 MHz, CDCl₃) δ ppm 1.61-1.66 (m, 1H), 1.73-1.90 (m, 3H), 1.99-2.01 (m, 1H), 2.06-2.08 (m, 1H), 3.77 (s, 3H), 5.12 (br s, 1H), 5.76 (d, 1H, J=10.0 Hz), 5.97 (d, 1H, J=9.8 Hz).

### Step 2. Formation of (S)/(R)-cyclohex-2-en-1-ol

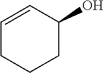

In an oven-dried round-bottom flask under argon, Pd₂(dba)₃.CHCl₃ (84 mg, 0.08 mmol, 4 mol %) and (R,R)-DACH-phenyl Trost ligand (112 mg, 0.16 mmol, 8 mol %) were dissolved in anhydrous DCM (17 mL). The resulting mixture was stirred at room temperature for 20 minutes. Degassed water (2 mL), a solution of cyclohex-2-en-1-yl methyl carbonate (316 mg, 2.02 mmol, 1.0 equiv.) in anhydrous DCM (1.2 mL) and KHCO₃ (284 mg, 2.83 mmol, 1.4 equiv.) were added and the resulting mixture was stirred at room temperature for 48 hours. The reaction mixture was diluted in water and the aqueous phase was extracted 3 times with DCM. The combined organic layers were washed with brine, dried over MgSO₄ and concentrated under vacuum. The crude was purified by chromatography on silica gel (hexanes:EtOAc 4:1 to 1:1), to obtain (S)-cyclohex-2-en-1-ol as a colorless oil (108 mg, 1.10 mmol, 54%). ¹H NMR (400 MHz, CDCl₃) δ ppm 1.57-1.61 (m, 2H), 1.69-1.74 (m, 1H), 1.84-1.89 (m, 1H), 1.95-1.99 (m, 1H), 2.00-2.04 (m, 1H), 4.20 (br s, 1H), 5.75 (dq, 1H, J=10.0 Hz, J=3.2 Hz), 5.83 (dtd, 1H, J=10.3 Hz, J=3.7 Hz, J=1.2 Hz).

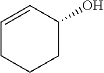

In an oven-dried round-bottom flask under argon, Pd₂(dba)₃.CHCl₃ (55 mg, 0.05 mmol, 4 mol %) and (S,S)-DACH-phenyl Trost ligand (73 mg, 0.11 mmol, 8 mol %) were dissolved in anhydrous DCM (11 mL). The resulting mixture was stirred at room temperature for 15 minutes. Degassed water (1.3 mL), a solution of cyclohex-2-en-1-yl methyl carbonate (207 mg, 1.32 mmol, 1.0 equiv.) in anhydrous DCM (1.0 mL) and KHCO₃ (186 mg, 1.86 mmol, 1.4 equiv.) were added and the resulting mixture was stirred at room temperature for 48 hours. The reaction mixture was diluted in water and the aqueous phase was extracted 3 times with DCM. The combined organic layers were washed with brine, dried over MgSO₄ and concentrated under vacuum. The crude was purified by chromatography on silica gel (hexanes:EtOAc 7:3 to 1:1), to obtain (R)-cyclohex-2-en-1-ol as a colorless oil (44 mg, 0.45 mmol, 34%). ¹H NMR (400 MHz, CDCl₃) δ ppm 1.57-1.61 (m, 2H), 1.69-1.74 (m, 1H), 1.84-1.89 (m, 1H), 1.95-1.99 (m, 1H), 2.00-2.04 (m, 1H), 4.20 (br s, 1H), 5.75 (dq, 1H, J=10.0 Hz, J=3.2 Hz), 5.83 (dtd, 1H, J=10.3 Hz, J=3.7 Hz, J=1.2 Hz).

### Preparation of 2-methylcyclohex-2-en-1-ol

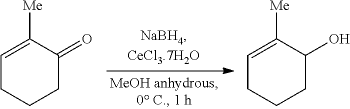 [SMILES: CC1=CCCCC1=O ; CC1=CCCCC1O]

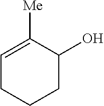

In an oven dried round-bottom flask under argon, 2-methylcyclohex-2-en-1-one (120 mg, 1.09 mmol, 1.0 equiv.) and CeCl₃.7H₂O (406 mg, 1.09 mmol, 1.0 equiv.) were dissolved in anhydrous methanol (11 mL, 0.1 M) and cooled down at 0° C. NaBH₄ (41 mg, 1.09 mmol, 1.0 equiv.) was added portion wise and the resulting mixture was stirred at 0° C. for 1 hour. The reaction was quenched with water and concentrated under vacuum. The residue was diluted in water and extracted 3 times with EtOAc. The combined organic layers were washed with brine, dried over MgSO₄ and concentrated under vacuum, to afford 2-methylcyclohex-2-en-1-ol as a colorless oil (78 mg, 0.70 mg, 64%). ¹H NMR (400 MHz, CDCl₃) δ ppm 1.53-1.66 (m, 3H), 1.73-1.78 (m, 5H), 1.91-2.02 (m, 2H), 3.98 (t, 1H, J=4.7 Hz), 5.54 (t, 1H, J=4.7 Hz); ¹³C NMR (100 MHz, CDCl₃) δ ppm 18.5, 20.9, 25.8, 32.5, 68.8, 125.9, 135.6.

### Preparation of Various Compounds

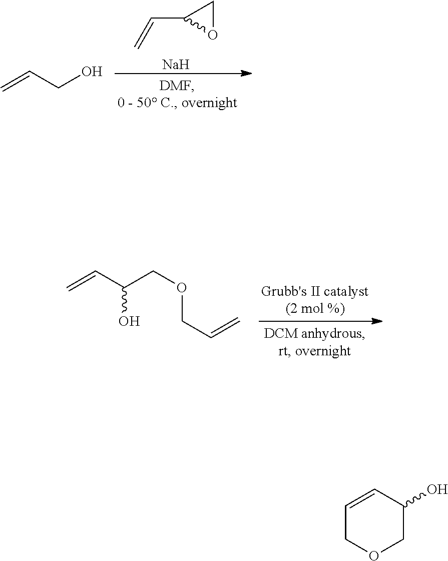

### Step 1. Formation of 1-(allyloxy)but-3-en-2-ol

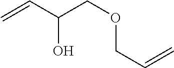

2-Vinyloxirane (500 μL, 6.21 mmol, 1.0 equiv.) and prop-2-en-1-ol (844 μL, 12.41 mmol, 2.0 equiv.) were dissolved in anhydrous DMF (12 mL, 0.5 M) and down at 0° C. NaH (60%, 496 mg, 12.41 mmol, 2.0 equiv.) was added portion wise and the resulting mixture was heated at 50° C. overnight. The reaction mixture was quenched with aqueous hydrochloric acid at 0° C. and stirred at 0° C. for 45 mins. The aqueous layer was extracted 3 times with EtOAc. The combined organic layers were washed with a 10% solution of LiCl and brine, dried over MgSO₄ and concentrated under vacuum. The crude was purified by chromatography on silica gel (hexanes:EtOAc 4:1), to obtain 1-(allyloxy)but-3-en-2-ol as a colorless oil (382 mg, 2.98 mmol, 48%). ¹H NMR (400 MHz, CDCl₃) δ ppm 2.24 (br s, 1H), 3.34 (dd, 1H, J=9.5 Hz, J=8.1 Hz), 3.52 (dd, 1H, J=9.5 Hz, J=3.4 Hz), 4.04 (dt, 1H, J=5.6 Hz, J=1.5 Hz), 4.31-4.35 (m, 1H), 5.20 (dt, 2H, J=10.5 Hz, J=1.7 Hz), 5.28 (dq, 1H, J=17.1 Hz, J=1.7 Hz), 5.37 (dt, 1H, J=17.4 Hz, J=1.7 Hz), 5.80-5.95 (m, 2H).

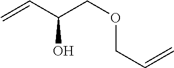

(S)-2-Vinyloxirane (400 mg, 5.71 mmol, 1.0 equiv.) and prop-2-en-1-ol (776 μL, 11.41 mmol, 2.0 equiv.) were dissolved in anhydrous DMF (11 mL, 0.5 M) and down at 0° C. NaH (60%, 457 mg, 11.41 mmol, 2.0 equiv.) was added portion wise and the resulting mixture was heated at 50° C. overnight. The reaction mixture was quenched with aqueous hydrochloric acid at 0° C. and stirred at 0° C. for 45 mins. The aqueous layer was extracted 3 times with EtOAc. The combined organic layers were washed with a 10% solution of LiCl and brine, dried over MgSO₄ and concentrated under vacuum. The crude was purified by chromatography on silica gel (hexanes:EtOAc 4:1), to obtain (S)-1-(allyloxy)but-3-en-2-ol as an orange oil (290 mg, 2.26 mmol, 40%). ¹H NMR (400 MHz, CDCl₃) δ ppm 2.24 (br s, 1H), 3.34 (dd, 1H, J=9.6 Hz, J=8.1 Hz), 3.51 (dd, 1H, J=9.6 Hz, J=3.4 Hz), 4.04 (dt, 1H, J=5.7 Hz, J=1.5 Hz), 4.31-4.35 (m, 1H), 5.20 (dt, 2H, J=10.5 Hz, J=1.7 Hz), 5.28 (dq, 1H, J=17.3 Hz, J=1.7 Hz), 5.37 (dt, 1H, J=17.3 Hz, J=1.7 Hz), 5.80-5.95 (m, 2H).

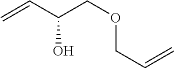

(R)-2-Vinyloxirane (300 mg, 4.28 mmol, 1.0 equiv.) and prop-2-en-1-ol (582 μL, 8.56 mmol, 2.0 equiv.) were dissolved in anhydrous DMF (8.6 mL, 0.5 M) and down at 0° C. NaH (60%, 342 mg, 8.56 mmol, 2.0 equiv.) was added portion wise and the resulting mixture was heated at 50° C. overnight. The reaction mixture was quenched with aqueous hydrochloric acid at 0° C. and stirred at 0° C. for 45 mins. The aqueous layer was extracted 3 times with EtOAc. The combined organic layers were washed with a 10% solution of LiCl and brine, dried over MgSO₄ and concentrated under vacuum. The crude was purified by chromatography on silica gel (hexanes:EtOAc 4:1), to obtain (R)-1-(allyloxy)but-3-en-2-ol as a yellow oil (143 mg, 1.11 mmol, 26%). ¹H NMR (400 MHz, CDCl₃) δ ppm 2.24 (br s, 1H), 3.34 (dd, 1H, J=9.6 Hz, J=8.1 Hz), 3.51 (dd, 1H, J=9.6 Hz, J=3.4 Hz), 4.04 (dt, 1H, J=5.7 Hz, J=1.5 Hz), 4.31-4.35 (m, 1H), 5.20 (dt, 2H, J=10.5 Hz, J=1.7 Hz), 5.28 (dq, 1H, J=17.3 Hz, J=1.7 Hz), 5.37 (dt, 1H, J=17.3 Hz, J=1.7 Hz), 5.80-5.95 (m, 2H).

### Step 2. Formation of 3,6-dihydro-2H-pyran-3-ol

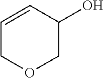

Following general procedure A and starting from 1-(allyloxy)but-3-en-2-ol (375 mg, 2.93 mmol, 1.0 equiv.), 3,6-dihydro-2H-pyran-3-ol was obtained as a yellow oil (112 mg, 1.12 mmol, 38%). ¹H NMR (400 MHz, CDCl₃) δ ppm 2.23 (br s, 1H), 3.73 (dd, 1H, J=11.7 Hz, J=2.4 Hz), 3.83 (dd, 1H, J=12.9 Hz, J=2.6 Hz), 3.96 (br s, 1H), 4.04 (dt, 1H, J=16.8 Hz, J=1.8 Hz), 4.15 (dd, 1H, J=16.9 Hz, J=1.6 Hz), 5.90 (dt, 1H, J=10.7 Hz, J=2.6 Hz), 5.97 (dt, 1H, J=10.2 Hz, J=2.6 Hz); ¹³C NMR (100 MHz, CDCl₃) δ ppm 62.9, 65.6, 71.1, 127.0, 130.2.

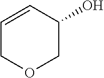

Following general procedure A and starting from (S)-1-(allyloxy)but-3-en-2-ol (289 mg, 2. mmol, 1.0 equiv.), (S)-3,6-dihydro-2H-pyran-3-ol was obtained as a brown oil (24 mg, 0.24 mmol, 11%). ¹H NMR (400 MHz, CDCl₃) δ ppm 2.23 (br s, 1H), 3.73 (dd, 1H, J=11.8 Hz, J=3.1 Hz), 3.84 (dd, 1H, J=11.8 Hz, J=2.7 Hz), 3.97 (br s, 1H), 4.06 (dq, 1H, J=16.9 Hz, J=2.1 Hz), 4.16 (dd, 1H, J=16.9 Hz, J=1.6 Hz), 5.92 (dt, 1H, J=10.7 Hz, J=2.6 Hz), 5.99 (dt, 1H, J=10.3 Hz, J=2.6 Hz); ¹³C NMR (100 MHz, CDCl₃) δ ppm 62.9, 65.6, 71.1, 127.0, 130.2.

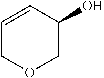

Following general procedure A and starting from (R)-1-(allyloxy)but-3-en-2-ol (142 mg, 1.11 mmol, 1.0 equiv.), (R)-3,6-dihydro-2H-pyran-3-ol was obtained as a yellow oil (49 mg, 0.49 mmol, 44%). ¹H NMR (400 MHz, CDCl₃) δ ppm 2.23 (br s, 1H), 3.73 (dd, 1H, J=11.8 Hz, J=3.1 Hz), 3.84 (dd, 1H, J=11.8 Hz, J=2.7 Hz), 3.97 (br s, 1H), 4.06 (dq, 1H, J=16.9 Hz, J=2.1 Hz), 4.16 (dd, 1H, J=16.9 Hz, J=1.6 Hz), 5.92 (dt, 1H, J=10.7 Hz, J=2.6 Hz), 5.99 (dt, 1H, J=10.3 Hz, J=2.6 Hz); ¹³C NMR (100 MHz, CDCl₃) δ ppm 62.9, 65.6, 71.1, 127.0, 130.2.

### Preparation of 1-(dimethylamino)but-3-en-2-ol and 4-(dimethylamino)but-2-en-1-ol

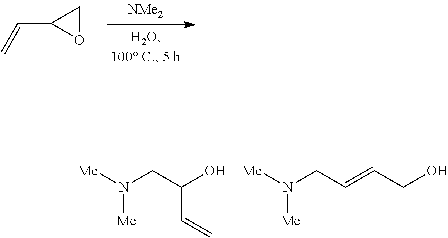

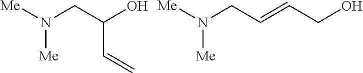

To a solution of dimethylamine (40% in water, 155.9 g, 1.14 mol, 4.0 equiv.) was added 2-vinyloxirane (20.0 g, 285 mmol, 1.0 equiv.). The resulting mixture was heated at 100° C. for 5 hours. Sodium hydroxide (70 g), was added to the mixture to reach pH 11-12. The aqueous phase was extracted 3 times with diethyl ether. The combined organic layers were dried over K₂CO₃. The crude was purified by distillation, to obtain a mixture of 1-(dimethylamino)but-3-en-2-ol and 4-(dimethylamino)but-2-en-1-ol as a yellow oil (13.4 g, 116 mmol, 41%). ¹H NMR (400 MHz, D20) δ ppm 2.16 (d, 12H, J=9.2 Hz), 2.39 (d, 2H, J=6.8 Hz), 2.85-2.89 (m, 1H), 3.53 (dd, 1H, J=8.2 Hz, J=7.8 Hz), 3.72 (dd, 1H, J=4.9 Hz, J=4.7 Hz), 4.21-4.25 (m, 1H), 5.15-5.32 (m, 3H), 5.78-5.83 (m, 2H).

### Example 4: Biological Procedures

LC-MS/MS Assay for Determining Choline TMA-Lyase Activity in E. coli MS 200-1

A frozen stock of E. coli MS 200-1 was streaked out onto a BHI agar plate inside an anaerobic chamber and incubated at 37° C. overnight. A single colony was inoculated into 5 mL of BHI supplemented with 1 mM choline chloride and grown anaerobically at 37° C. The starter culture was inoculated (2%) into fresh BHI containing 1 mM of choline chloride-(trimethyl-d9). The bacterial culture was distributed among the wells of a 96-well plate. Inhibitors were dissolved in 50 mM potassium phosphate buffer, pH 7.4, and aliquoted to the wells so that the final volume of each well was 200 μL. Plates were covered with aluminum seals and incubated at 37° C. for 3.5 h inside an anaerobic chamber. An aliquot of each culture was diluted (up to 3750-fold) in a mixture composed of 95% acetonitrile and 5% of 100 mM ammonium formate buffer (final formic acid concentration of 0.02%) and analyzed by LC-MS/MS for d9-TMA and d9-choline.

In Vitro Assay for Measuring CutC Activity in the Presence of Inhibitors

The activity of CutC was coupled to the reduction of acetaldehyde by NADH-dependent yeast alcohol dehydrogenase (YADH). Assays were conducted in a buffer of 50 mM potassium phosphate, pH 8.0, 50 mM KCl and were set up in an anaerobic chamber kept at 20° C. in 1.5 mL polypropylene Eppendorf tubes. CutD (40 μM) and sodium dithionite (150 μM) were incubated for 20 min prior to the addition of 200 μM of S-(5′-adenosyl)-L-methionine (SAM as a p-toluenesulfonate salt) and 20 μM of CutC monomer in a total reaction mixture of 50 L. Glycyl radical formation was carried out for 1 h, after which the activated CutC mixture was immediately diluted 20-fold into phosphate buffer. An aliquot of the diluted mixture was added to a master mix containing NADH (200 μM) and YADH (0.4 μM) such that the final concentration of CutC monomer was 5 nM (after addition of inhibitor/buffer and choline). The master mix was distributed to the wells of a 96-well plate (180 μL per well), to which was added either inhibitor or buffer (10 μL per well), followed by choline (10 μL per well). The final concentration of choline was 200 μM (˜K_{M} value).

Following the addition of choline, the absorbance of NADH at 340 nm was immediately recorded, with measurements taken every 20 s for 10 min. Initial rates were calculated from absorbance measurements that decreased linearly. Pathlengths were corrected to 1 cm and absorbance values were converted to concentrations assuming ε₃₄₀=6,220 M⁻¹ cm⁻¹ for NADH. The rate from assays containing no substrate was subtracted from assays containing substrate to account for background activity.

Determining Choline TMA-Lyase Activity in Cut Gene Cluster-Harboring Bacteria

Frozen stocks of Escherichia coli MS 69-1, Proteus mirabilis ATCC 29906 and Klebsiella sp. MS 92-3 were streaked out onto BHI agar plates inside an anaerobic chamber and incubated at 37° C. overnight. A single colony of each strain was inoculated into 5 mL of BHI supplemented with 1 mM choline chloride, which was grown anaerobically at 37° C. The same conditions were used for culturing C. sporogenes ATCC 15579 except, instead of BHI, an RCM agar plate and RCM liquid broth were used. For each strain, BHI (or RCM) containing 1 mM of choline chloride-(trimethyl-d9) was inoculated with 2% of the saturated bacterial starter culture. Cultures were distributed among the wells of a 96-well plate and inhibitors, dissolved in 50 mM potassium phosphate buffer, pH 7.4, was added to the samples in the wells. The final volume of each well was 200 μL. Plates were covered with aluminum seals and incubated at 37° C. inside a vinyl anaerobic chamber for 3.5 hours. An aliquot of each culture was diluted (up to 3750-fold) in a mixture composed of 95% acetonitrile and 5% of 100 mM ammonium formate buffer (with a final formic acid concentration of 0.02%) and analyzed by LC-MS/MS for d9-TMA and d9-choline.

Determining Choline TMA-Lyase Activity in a Fecal Suspension Ex Vivo

A frozen fecal sample from a healthy individual was diluted 1:10 (wt/v) in sterile anaerobic Mega media containing 1 mM of choline chloride-(trimethyl-d₉). A 190 μL aliquot of the resulting supernatant was transferred to a 96-well plate containing 10 μL of different concentrations of inhibitor, and the mixtures were incubated for 18 h at 37° C. An aliquot of each culture was diluted 3750-fold in a mixture composed of 95% acetonitrile and 5% of 100 mM ammonium formate buffer (with a final formic acid concentration of 0.02%) and analyzed by LC-MS/MS for d₉-TMA and d₉-choline.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound of Formula (I), Formula (Ia), Formula (II), or Formula (III): 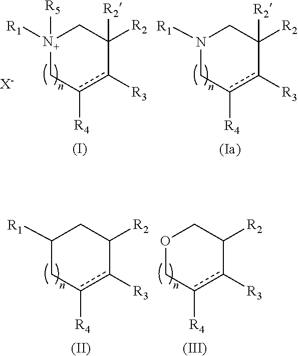
or a pharmaceutically acceptable salt thereof, wherein [symbol]
is a single bond or a double bond;
R₁ is alkyl, cycloalkyl, cycloalkyl(alkyl), heterocyclyl, heterocyclylalkyl, aryl, heteroaryl, aralkyl, or heteroaralkyl;
R₂ is hydroxyl, alkoxy, thio, alkylthio, or halo;
R₂′ is H or D;
R₃ is H, alkyl, or cycloalkyl;
R₄ is H, alkyl, or cycloalkyl;
or R₃ and R₄, taken together with the carbon atoms to which they are attached, form a cycloalkyl or cycloalkenyl ring;
R₅ is alkyl;
X is a counter ion;
n is 0, 1, 2, or 3,
provided the compound is not

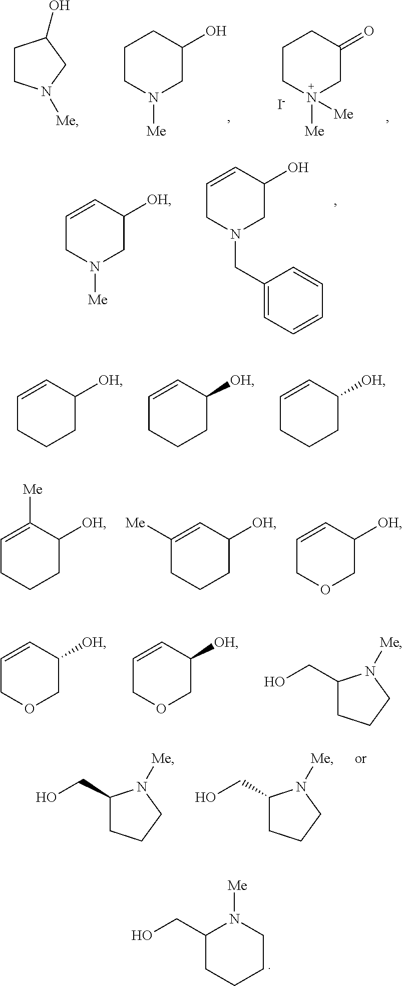

2. (canceled)

3. The compound of claim 1, wherein the compound is represented by formula (I′): 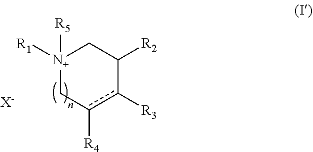
or a pharmaceutically acceptable salt thereof, wherein [symbol]
is a single bond or a double bond;
R₁ is alkyl or cycloalkyl;
R₂ is hydroxyl, alkoxy, thio, alkylthio, or halo;
R₃ is H, alkyl, or cycloalkyl;
R₄ is H, alkyl, or cycloalkyl;
or R₃ and R₄, taken together with the carbon atoms to which they are attached, form a cycloalkyl or cycloalkenyl ring;
R₅ is alkyl;
X is a counter ion; and
n is 0, 1, 2, or 3.

4. The compound of claim 1, wherein the compound is compound of Formula (I); and if R₁ is methyl, R₂ is hydroxyl, [symbol] is a double bond, R₃ is H, R₄, is H, and R₅ is allyl, then n is not 1.

5. The compound of claim 1, wherein R⁵ is methyl.

6. The compound of claim 1, wherein X is halo.

7. The compound of claim 1, wherein the compound is represented by Formula (Ia′): 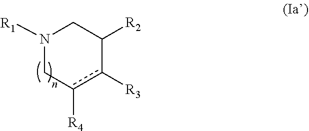
or a pharmaceutically acceptable salt thereof, wherein [symbol]
is a single bond or a double bond;
R₁ is alkyl or cycloalkyl;
R₂ is hydroxyl, alkoxy, thio, alkylthio, or halo;
R₃ is H, alkyl, or cycloalkyl;
R₄ is H, alkyl, or cycloalkyl;
or R₃ and R₄, taken together with the carbon atoms to which they are attached, form a cycloalkyl or cycloalkenyl ring; and
n is 0, 1, 2, or 3,
provided that if R₁ is methyl, R₂ is hydroxyl, [symbol] is a double bond, R₃ is H, and R₄ is H, then n is not 1.

8. The compound of claim 1, wherein the compound is represented by Formula (II): 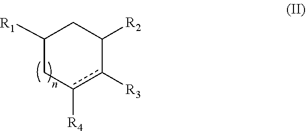
or a pharmaceutically acceptable salt thereof, wherein [symbol]
is a single bond or a double bond;
R₁ is alkyl or cycloalkyl;
R₂ is hydroxyl, alkoxy, thio, alkylthio, or halo;
R₃ is H, alkyl, or cycloalkyl;
R₄ is H, alkyl, or cycloalkyl;
or R₃ and R₄, taken together with the carbon atoms to which they are attached, form a cycloalkyl or cycloalkenyl ring; and
n is 0, 1, 2, or 3.

9. The compound of claim 1, wherein R₁ is alkyl.

10. (canceled)

11. The compound of claim 1, wherein R₁ is cycloalkyl.

12-16. (canceled)

17. The compound of claim 1, wherein R₁ is (cycloalkyl)alkyl, heterocyclylalkyl, aralkyl, or heteroaryl.

18-20. (canceled)

21. The compound of claim 1, wherein R₁ is substituted with alkyl, carboxyl, hydroxyl, hydroxyalkyl, alkoxy, halogen, acyl, acyloxy, amino, aminoalkyl, or cyano.

22-26. (canceled)

27. The compound of claim 1, wherein the compound is represented by Formula (III): 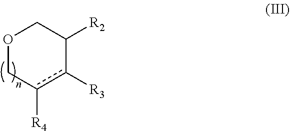
or a pharmaceutically acceptable salt thereof, wherein [symbol]
is a single bond or a double bond;
R₂ is hydroxyl, alkoxy, thio, alkylthio, or halo;
R₃ is H, alkyl, or cycloalkyl;
R₄ is H, alkyl, or cycloalkyl;
or R₃ and R₄, taken together with the carbon atoms to which they are attached, form a cycloalkyl or cycloalkenyl ring; and
n is 0, 1, 2, or 3.

28. The compound of claim 1, wherein R₂ is hydroxyl.

29-31. (canceled)

32. The compound of claim 1, wherein [symbol] is a double bond.

33. The compound of claim 1, wherein R₃ is H or alkyl.

34. (canceled)

35. The compound of claim 1, wherein R₄ is H.

36. The compound of claim 1, wherein the compound is: 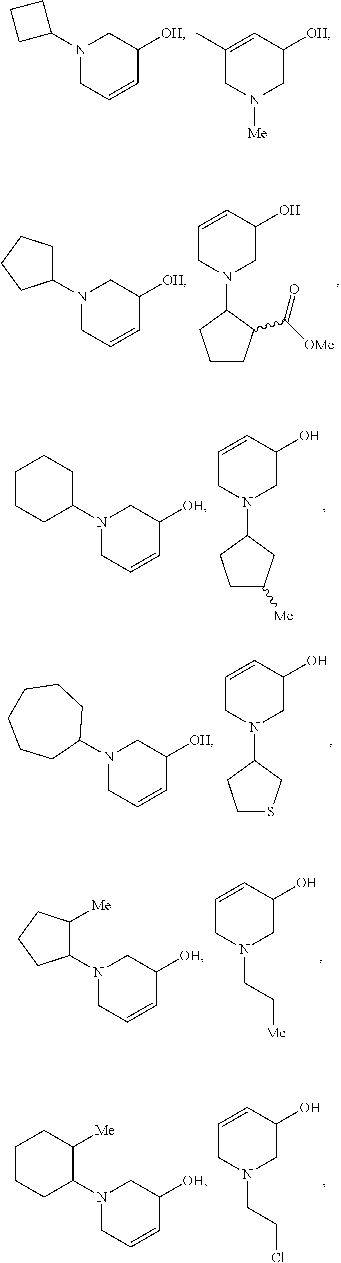 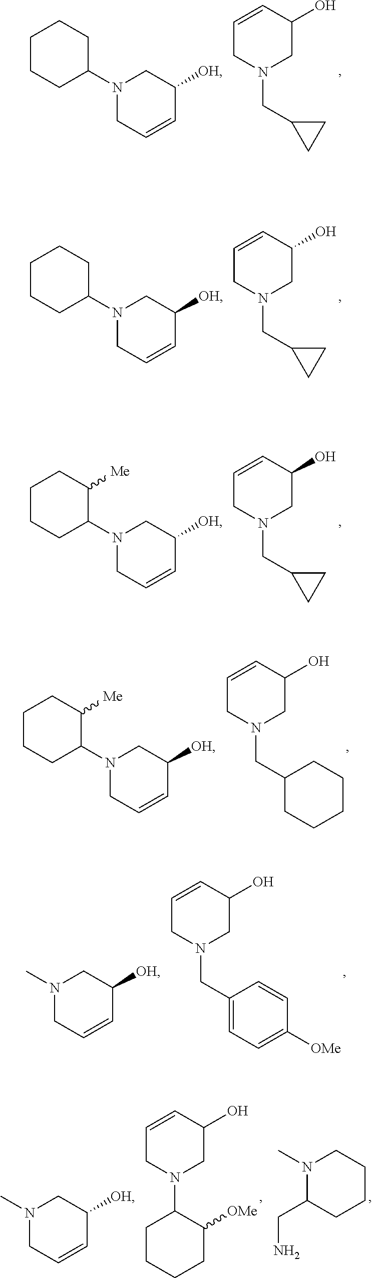 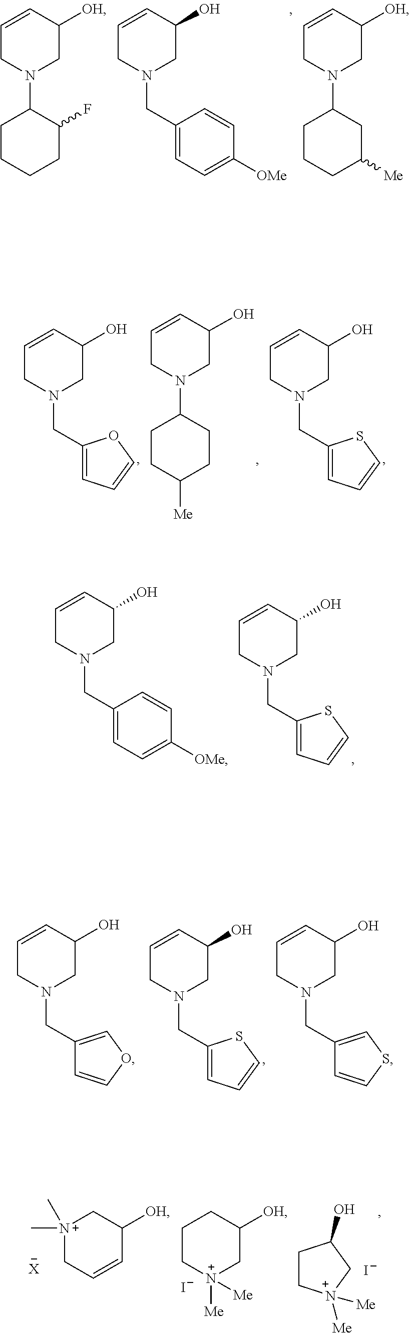 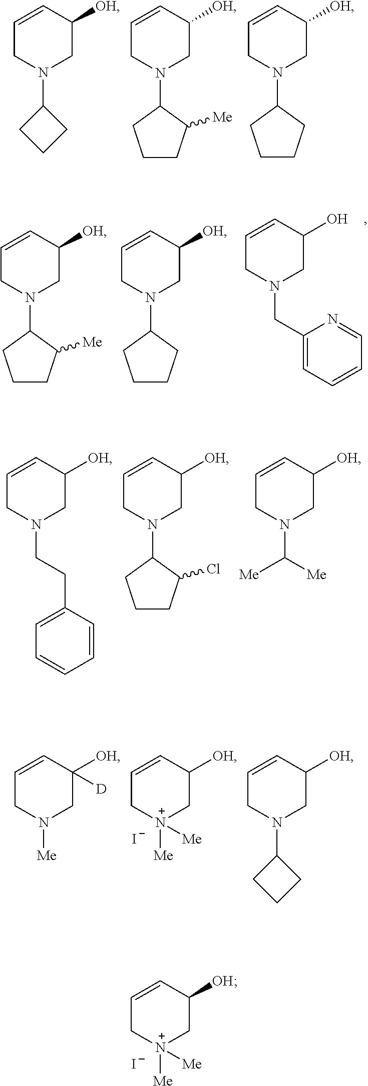
a pharmaceutically acceptable salt thereof.

37. (canceled)

38. (canceled)

39. A pharmaceutical composition comprising a compound of claim 1 and at least one pharmaceutically acceptable excipient.

40-65. (canceled)

66. A method for:
i) treating a choline disorder in a subject; or
ii) inhibiting choline metabolism in a subject,
comprising administering to the subject an effective amount of either: a compound of claim 1 or a pharmaceutically acceptable salt thereof; or a compound selected from 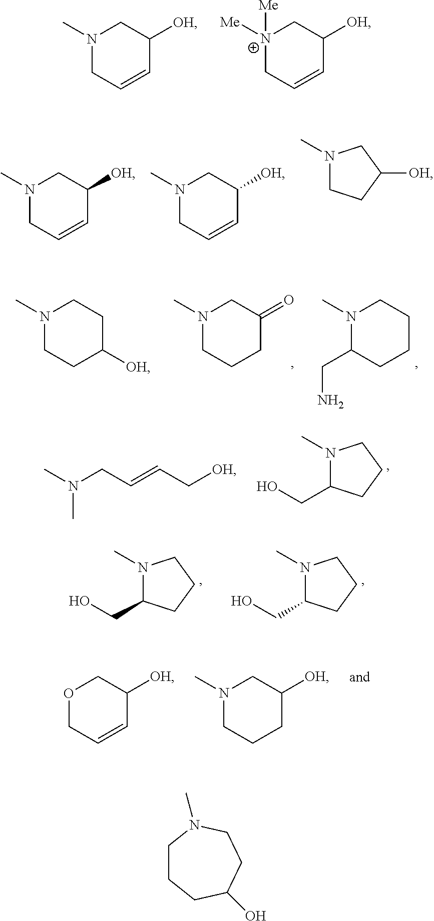
or a pharmaceutically acceptable salt thereof.

67-108. (canceled)

109. A method for inhibiting choline metabolism in a cell, comprising contacting the cell with an effective amount of either: a compound of claim 1 or a pharmaceutically acceptable salt thereof: or a compound selected from 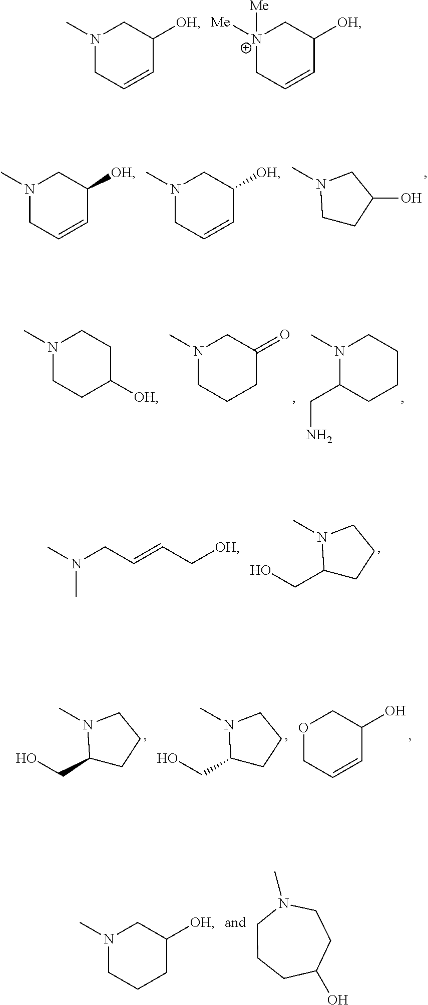
or a pharmaceutically acceptable salt thereof.

110-125. (canceled)
